SEQUENCE LISTING (42 sequences)

SEQ ID NO: 1 - DNA, 21 bp, synthetic construct
  misc_feature 1..21: note = "A class" CpG oligonucleotide
  source 1..21: mol_type = other DNA
ggggacgacgtcgtggggggg

SEQ ID NO: 2 - DNA, 21 bp, synthetic construct
  misc_feature 1..21: note = "A class" CpG oligonucleotide
  source 1..21: mol_type = other DNA
ggggacgacgtcgtggggggg

SEQ ID NO: 3 - DNA, 21 bp, synthetic construct
  misc_feature 1..21: note = "B class" CpG oligonucleotide
  source 1..21: mol_type = other DNA
tcgtcgtttttcggtgctttt

SEQ ID NO: 4 - DNA, 21 bp, synthetic construct
  misc_feature 1..21: note = "B class" CpG oligonucleotide
  source 1..21: mol_type = other DNA
tcgtcgtttttcggtcgtttt

SEQ ID NO: 5 - DNA, 24 bp, synthetic construct
  misc_feature 1..24: note = "B class" CpG oligonucleotide
  source 1..24: mol_type = other DNA
tcgtcgttttgtcgttttgtcgtt

SEQ ID NO: 6 - DNA, 24 bp, synthetic construct
  misc_feature 1..24: note = "B class" CpG oligonucleotide
  source 1..24: mol_type = other DNA
tcgtcgtttcgtcgttttgtcgtt

SEQ ID NO: 7 - DNA, 24 bp, synthetic construct
  misc_feature 1..24: note = "B class" CpG oligonucleotide
  source 1..24: mol_type = other DNA
tcgtcgttttgtcgtttttttcga

SEQ ID NO: 8 - DNA, 21 bp, synthetic construct
  misc_feature 1..21: note = B-Class oligonucleotide
  source 1..21: mol_type = other DNA
tcgtcgtttttcggtgctttt

SEQ ID NO: 9 - DNA, 21 bp, synthetic construct
  misc_feature 1..21: note = B-Class oligonucleotide
  source 1..21: mol_type = other DNA
tcgtcgtttttcggtcgtttt

SEQ ID NO: 10 - DNA, 24 bp, synthetic construct
  misc_feature 1..24: note = B-Class oligonucleotide
  source 1..24: mol_type = other DNA
tcgtcgttttgtcgttttgtcgtt

SEQ ID NO: 11 - DNA, 24 bp, synthetic construct
  misc_feature 1..24: note = B-Class oligonucleotide
  source 1..24: mol_type = other DNA
tcgtcgtttcgtcgttttgtcgtt

SEQ ID NO: 12 - DNA, 24 bp, synthetic construct
  misc_feature 1..24: note = B-Class oligonucleotide
  source 1..24: mol_type = other DNA
tcgtcgttttgtcgtttttttcga

SEQ ID NO: 13 - DNA, 22 bp, synthetic construct
  misc_feature 1..22: note = C class CpG Oligonucleotide
  source 1..22: mol_type = other DNA
tcgcgtcgttcggcgcgcgccg

SEQ ID NO: 14 - DNA, 23 bp, synthetic construct
  misc_feature 1..23: note = C class CpG Oligonucleotide
  source 1..23: mol_type = other DNA
tcgtcgacgttcggcgcgcgccg

SEQ ID NO: 15 - DNA, 21 bp, synthetic construct
  misc_feature 1..21: note = C class CpG Oligonucleotide
  source 1..21: mol_type = other DNA
tcggacgttcggcgcgcgccg

SEQ ID NO: 16 - DNA, 19 bp, synthetic construct
  misc_feature 1..19: note = C class CpG Oligonucleotide
  source 1..19: mol_type = other DNA
tcggacgttcggcgcgccg

SEQ ID NO: 17 - DNA, 20 bp, synthetic construct
  misc_feature 1..20: note = C class CpG Oligonucleotide
  source 1..20: mol_type = other DNA
tcgcgtcgttcggcgcgccg

SEQ ID NO: 18 - DNA, 20 bp, synthetic construct
  misc_feature 1..20: note = C class CpG Oligonucleotide
  source 1..20: mol_type = other DNA
tcgacgttcggcgcgcgccg

SEQ ID NO: 19 - DNA, 18 bp, synthetic construct
  misc_feature 1..18: note = C class CpG Oligonucleotide
  source 1..18: mol_type = other DNA
tcgacgttcggcgcgccg

SEQ ID NO: 20 - DNA, 18 bp, synthetic construct
  misc_feature 1..18: note = C class CpG Oligonucleotide
  source 1..18: mol_type = other DNA
tcgcgtcgttcggcgccg

SEQ ID NO: 21 - DNA, 22 bp, synthetic construct
  misc_feature 1..22: note = C class CpG Oligonucleotide
  source 1..22: mol_type = other DNA
tcgcgacgttcggcgcgcgccg

SEQ ID NO: 22 - DNA, 22 bp, synthetic construct
  misc_feature 1..22: note = C class CpG Oligonucleotide
  source 1..22: mol_type = other DNA
tcgtcgttttcggcgcgcgccg

SEQ ID NO: 23 - DNA, 22 bp, synthetic construct
  misc_feature 1..22: note = C class CpG Oligonucleotide
  source 1..22: mol_type = other DNA
tcgtcgttttcggcggccgccg

SEQ ID NO: 24 - DNA, 24 bp, synthetic construct
  misc_feature 1..24: note = C class CpG Oligonucleotide
  source 1..24: mol_type = other DNA
tcgtcgttttacggcgccgtgccg

SEQ ID NO: 25 - DNA, 23 bp, synthetic construct
  misc_feature 1..23: note = C class CpG Oligonucleotide
  source 1..23: mol_type = other DNA
tcgtcgttttcggcgcgcgccgt

SEQ ID NO: 26 - DNA, 22 bp, synthetic construct
  misc_feature 1..22: note = C-Class oligonucleotides
  source 1..22: mol_type = other DNA
tcgcgtcgttcggcgcgcgccg

SEQ ID NO: 27 - DNA, 23 bp, synthetic construct
  misc_feature 1..23: note = C-Class oligonucleotide
  source 1..23: mol_type = other DNA
tcgtcgacgttcggcgcgcgccg

SEQ ID NO: 28 - DNA, 21 bp, synthetic construct
  misc_feature 1..21: note = C-Class oligonucleotide
  source 1..21: mol_type = other DNA
tcggacgttcggcgcgcgccg

SEQ ID NO: 29 - DNA, 19 bp, synthetic construct
  misc_feature 1..19: note = C-Class oligonucleotide
  source 1..19: mol_type = other DNA
tcggacgttcggcgcgccg

SEQ ID NO: 30 - DNA, 20 bp, synthetic construct
  misc_feature 1..20: note = C-Class oligonucleotide
  source 1..20: mol_type = other DNA
tcgcgtcgttcggcgcgccg

SEQ ID NO: 31 - DNA, 20 bp, synthetic construct
  misc_feature 1..20: note = C-Class oligonucleotide
  source 1..20: mol_type = other DNA
tcgacgttcggcgcgcgccg

SEQ ID NO: 32 - DNA, 18 bp, synthetic construct
  misc_feature 1..18: note = C-Class oligonucleotide
  source 1..18: mol_type = other DNA
tcgacgttcggcgcgccg

SEQ ID NO: 33 - DNA, 18 bp, synthetic construct
  misc_feature 1..18: note = C-Class oligonucleotide
  source 1..18: mol_type = other DNA
tcgcgtcgttcggcgccg

SEQ ID NO: 34 - DNA, 22 bp, synthetic construct
  misc_feature 1..22: note = C-Class oligonucleotide
  source 1..22: mol_type = other DNA
tcgcgacgttcggcgcgcgccg

SEQ ID NO: 35 - DNA, 22 bp, synthetic construct
  misc_feature 1..22: note = C-Class oligonucleotide
  source 1..22: mol_type = other DNA
tcgtcgttttcggcgcgcgccg

SEQ ID NO: 36 - DNA, 22 bp, synthetic construct
  misc_feature 1..22: note = C-Class oligonucleotide
  source 1..22: mol_type = other DNA
tcgtcgttttcggcggccgccg

SEQ ID NO: 37 - DNA, 24 bp, synthetic construct
  misc_feature 1..24: note = C-Class oligonucleotide
  source 1..24: mol_type = other DNA
tcgtcgttttacggcgccgtgccg

SEQ ID NO: 38 - DNA, 23 bp, synthetic construct
  misc_feature 1..23: note = C-Class oligonucleotide
  source 1..23: mol_type = other DNA
tcgtcgttttcggcgcgcgccgt

SEQ ID NO: 39 - DNA, 23 bp, synthetic construct
  misc_feature 1..23: note = P class CpG oligonucleotide
  source 1..23: mol_type = other DNA
tcgtcgacgatcggcgcgcgccg

SEQ ID NO: 40 - DNA, 23 bp, synthetic construct
  misc_feature 1..23: note = P class CpG oligonucleotide
  source 1..23: mol_type = other DNA
tcgtcgacgatcggcgcgcgccg

SEQ ID NO: 41 - protein, 950 aa, synthetic construct
  REGION 1..950: note = Enzymatically inactive fragment of SCP
MAKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

SEQ ID NO: 42 - protein, 949 aa, synthetic construct
  REGION 1..949: note = Enzymatically inactive fragment of SCP
AKTADTPATSKATIRDLNDPSQVKTLQEKAGKGAGTVVAVIAAGFDKNHEAWRLTDKAKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVTVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTAMSAPLVAGIMGLLQEQYETQYPDMTPSERLDLAKKVLMSSATALYDEDEKAYFSPRQQGAGAVDAKKASAATMYVTDKDNTSSKVHLNNVSDKFEVTVTVHNKSDKPQELYYQATVQTDKVDGKHFALAPKALYETSWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNKPEQ

(19) United States
(12) Patent Application Publication
(10) Pub. No.: US 2025/0381259 A1
(43) Pub. Date: Dec. 18, 2025

(54) Immunogenic Compositions Comprising Conjugated Capsular Saccharide Antigens and Uses Thereof

(71) Applicant: PFIZER INC., New York, NY (US)

(72) Inventors: Annaliesa Sybil ANDERSON, Upper Saddle River, NJ (US); Caitlyn GALLAGHER, Ringwood, NJ (US); Jianxin GU, Paramus, NJ (US); Isis KANEVSKY, New York, NY (US); Jin-Hwan KIM, Pearl River, NY (US); Justin Keith MORAN, West Nyack, NY (US); Suddham SINGH, Monroe, NY (US); Abhishek Ravindra VARTAK, Westwood, NJ (US); YuYing YANG, Stamford, CT (US)

(21) Appl. No.: 18/727,436

(22) PCT Filed: Jan. 10, 2023
(86) PCT No.: PCT/IB2023/050202

§ 371 Date: Jul. 9, 2024

Related U.S. Application Data
(60) Provisional application No. 63/299,101, filed on Jan. 13, 2022.

(51) Int. Cl. A61K 39/09; A61K 39/00; A61K 47/64; A61P 37/04

(52) U.S. Cl. CPC A61K 39/092; A61K 47/6415; A61K 47/646; A61P 37/04; A61K 2039/6037; A61K 2039/627

(57) ABSTRACT

The present invention relates to new conjugated capsular saccharide antigens (glycoconjugates), immunogenic compositions comprising said glycoconjugates and uses thereof.

### FIELD OF THE INVENTION

The present invention relates to new conjugated capsular saccharide antigens (glycoconjugates), immunogenic compositions comprising said glycoconjugates and uses thereof. Immunogenic compositions of the present invention will typically comprise glycoconjugates, wherein the saccharides are derived from bacterial capsular polysaccharide antigens, in particular a capsular polysaccharide derived from pathogenic bacteria. The invention also relates to vaccination of human subjects, in particular infants and elderly, against infections using said glycoconjugates.

### BACKGROUND OF THE INVENTION

Both Gram-positive and Gram-negative bacteria may produce an extracellular compartment, the capsule, which covers the bacterial cell and often prevents the reaction of underlying cell surface antigens with their homologous antibodies. Capsules are found in several bacteria of medical importance, especially in extraintestinal and invasive strains. Bacterial capsules are largely composed of polysaccharides. They form a gelatinous mass around the cell. Capsular polysaccharides are important immunogens and has led to them being an important component in the design of vaccines. They have proved useful in eliciting immune responses especially when linked to carrier proteins.

The approach to increasing immunogenicity of poorly immunogenic molecules by conjugating these molecules to “carrier” molecules has been utilized successfully for decades (see, e.g., Goebel et al. (1939) J. Exp. Med. 69: 53). For example, many immunogenic compositions have been described in which purified capsular polymers have been conjugated to carrier proteins to create more effective immunogenic compositions by exploiting this “carrier effect.” Schneerson et al. (1984) Infect. Immun. 45: 582-591). Conjugation has also been shown to bypass the poor antibody response usually observed in infants when immunized with a free polysaccharide (Anderson et al. (1985) J. Pediatr. 107: 346; Insel et al. (1986) J. Exp. Med. 158: 294).

Conjugates have been successfully generated using various cross-linking or coupling reagents, such as homobifunctional, heterobifunctional, or zero-length crosslinkers. Many methods are currently available for coupling immunogenic molecules, such as saccharides, proteins, and peptides, to peptide or protein carriers. Most methods create amine, amide, urethane, isothiourea, or disulfide bonds, or in some cases thioethers. A disadvantage to the use of cross-linking or coupling reagents which introduce reactive sites into the side chains of reactive amino acid molecules on carrier and/or immunogenic molecules is that the reactive sites, if not neutralized, are free to react with any unwanted molecule either in vitro (thus potentially adversely affecting the functionality or stability of the conjugates) or in vivo (thus posing a potential risk of adverse events in persons or animals immunized with the preparations). Such excess reactive sites can be reacted or “capped”, so as to inactivate these sites, utilizing various known chemical reactions, but these reactions may be otherwise disruptive to the functionality of the conjugates.

Thus, there remains a need for new glycoconjugates appropriately capped and methods to prepare said conjugates, such that the functionality is preserved, and the conjugate retains the ability to elicit the desired immune response.

The present inventors have found a new and efficacious method to generate glycoconjugates. The method allows for generating conjugates with very low free saccharide and a good yield. Furthermore, the present inventors have found that some Streptococcus pneumoniae serotypes (e.g. 35B and 29 polysaccharides) pose particular challenges to generate conjugates due to their unique structure. There is a need for immunogenic Streptococcus pneumoniae serotypes polysaccharide-carrier protein conjugates and an improved process for making the same.

### SUMMARY OF THE INVENTION

In an aspect, the present invention pertains to a method of making a capsular saccharide glycoconjugate, comprising the steps of:
(a) reacting an isolated capsular saccharide with a carbonic acid derivative and an azido linker in an aprotic solvent to produce an activated azido saccharide,
(b) reacting a carrier protein with an agent bearing an N-Hydroxysuccinimide (NHS) moiety and an alkyne group where the NHS moiety reacts with the amino groups to form an amide linkage thereby obtaining an alkyne functionalized carrier protein,
(c) reacting the activated azido saccharide of step (a) with the activated alkyne-carrier protein of step (b) by Cu⁺¹ mediated azide-alkyne cycloaddition reaction to form a glycoconjugate.

In an aspect, the isolated saccharide is sized before the activation step (a).

In an aspect, the carbonic acid derivative is selected from the group consisting of 1,1′-carbonyldiimidazole (CDI), 1,1′-carbonyl-di-(1,2,4-triazole) (CDT), disuccinimidyl carbonate (DSC) and N-hydroxysuccinimidyl chloroformate.

In an aspect, said azido linker is a compound of formula (I),

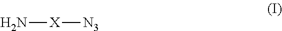

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂;
where n is selected from 1 to 10 and m is selected from 1 to 4.

In an aspect, said agent bearing an N-Hydroxysuccinimide (NHS) moiety and an alkyne group is an agent bearing an N-Hydroxysuccinimide (NHS) moiety and a terminal alkyne.

In an aspect, step a) comprises reacting the capsular saccharide with a carbonic acid derivative followed by reacting the carbonic acid derivative-activated capsular saccharide with an azido linker in an aprotic solvent to produce an activated azido capsular saccharide.

In an aspect, the conjugation reaction c) is carried out in aqueous buffer in the presence of copper (I) as catalyst.

In an aspect, following step c), the method further comprises a step of capping the unreacted azido groups remained in the conjugate with an azido group capping agent.

In an aspect, following step c), the method further comprises a step of capping the unreacted alkyne groups remained in the conjugate with an alkyne group capping agent.

In an aspect, the present invention pertains to a capsular saccharide glycoconjugate produced according said methods.

In a further aspect, the present invention pertains to a capsular saccharide glycoconjugate comprising a capsular saccharide covalently conjugated to a carrier protein (CP) through a spacer and having the general formula (VII):

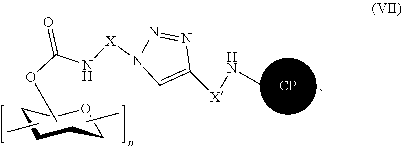

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)ₙ, and O(CH₂CH₂O)ₘCH₂CH₂; where n′ is selected from 1 to 10 and m is selected from 1 to 4, and wherein X is selected from the group consisting of CH₂O(CH₂)_{n′}CH₂C═O, CH₂O(CH₂CH₂O)_{m′}(CH₂)_{n″}CH₂C═O, where n″ is selected from 0 to 10 and m′ is selected from 0 to 4.

In a particular aspect, the invention is directed to a capsular saccharide glycoconjugate comprising a capsular saccharide covalently conjugated to a carrier protein (CP) through a spacer and having the general formula (VII), wherein X is CH₂(CH₂)_{n′}, where n′ is 2 and wherein X is CH₂O(CH₂)_{n′}CH₂C═O where n″ is 1.

In a particular aspect, the invention pertains to a capsular saccharide glycoconjugate comprising a capsular saccharide covalently conjugated to a carrier protein (CP) through a spacer and having the general formula (VIII),

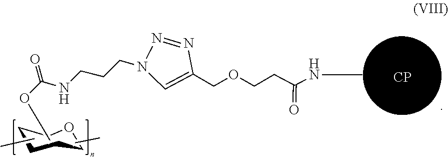

### FIGURES

FIG. 1 shows a repeating polysaccharide structure of the S. pneumoniae serotype 35B capsular polysaccharide.

FIG. 2 shows a repeating polysaccharide structure of the S. pneumoniae serotype 29 capsular polysaccharide.

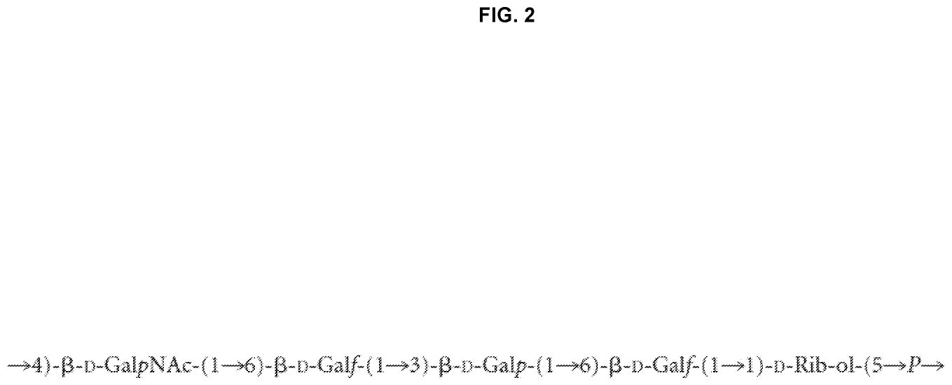

FIG. 3 shows a general scheme for the preparation of glycoconjugate of the invention prepared using click chemistry and using 3-Azido-1-propylamine as azido linker. CP=Carrier Protein, CDI=1,1′-carbonyldiimidazole.

### DETAILED DESCRIPTION OF THE INVENTION

The present inventors have developed new polysaccharide-carrier protein conjugates and processes for making these conjugates. The method to generate glycoconjugates has been found to allow for producing glycoconjugates with very low free saccharide and a good yield.

Furthermore, the present inventors have found that Streptococcus pneumoniae serotypes 35B and 29 polysaccharides pose particular challenges to generate conjugates. Streptococcus pneumoniae serotypes 35B and 29 polysaccharides have been found to be cleaved during activation with periodate, which is the classical oxidant used in the commonly used reductive amination process. It appears that periodate oxidation occurs on the backbone of serotypes 35B and 29 polysaccharides and cleaves the mannitol or ribitol, leading to size reduction. This leads to several issues, including difficulties to obtain activated serotypes 35B and 29 polysaccharides of a certain size. The activation with periodate results in a decrease in polysaccharide Mw drastically limiting the effective activation range.

WO 2020/247299 suggests decreasing the amount of periodate and using a certain temperature range for the conjugation reaction in order to allegedly improve serotype 35B conjugates attributes. However, such a solution has been found to lead to low yield and/or high free saccharide. The present inventors have developed new Streptococcus pneumoniae serotypes 35B and 29 polysaccharide-carrier protein conjugates and improved processes for making these conjugates which does not suffer from these deficiencies.

### 1. Glycoconjugates of the Invention

The present invention is directed in part to conjugated bacterial capsular saccharide antigens (also named glycoconjugates). For the purpose of the invention the term ‘glycoconjugate’ indicates a capsular saccharide (in particular a bacterial capsular saccharide) linked covalently to a carrier protein.

### 1.1 Capsular Saccharide of the Invention

The term “saccharide” throughout this specification may indicate polysaccharide or oligosaccharide and includes both. In an embodiment, saccharide of the invention may be oligosaccharides. Oligosaccharides have a low number of repeat units (typically 5-15 repeat units) and are typically derived synthetically or by hydrolysis of polysaccharides. Preferably though, all of the saccharides of the present invention and in the immunogenic compositions of the present invention are polysaccharides. High molecular weight polysaccharides are able to induce certain antibody immune responses due to the epitopes present on the antigenic surface. The isolation and purification of high molecular weight capsular polysaccharides is preferably contemplated for use in the conjugates, compositions and methods of the present invention. Therefore, in a preferred embodiment of the present invention, the saccharide is a polysaccharide.

Preferably, the saccharide used in the present invention is a bacterial capsular saccharide (also named ‘capsular saccharide’ herein). Capsules are found in several bacteria of medical importance. Bacterial capsules are largely composed of polysaccharides. Capsular saccharides are prepared by standard techniques known to those of ordinary skill in the art.

In a most preferred embodiment of the present invention, the saccharide is a S. pneumoniae capsular polysaccharide.

In an embodiment, the capsular saccharide used in the present invention is a synthetic carbohydrate.

In a preferred embodiment though, the source of bacterial capsular saccharide according to this invention can be bacterial cells. Bacterial strains which can be used as source of capsular saccharide may be obtained from established culture collections (such as for example from the American Type Culture Collection (ATCC, Manassas, VA USA) or the Streptococcal Reference Laboratory (Centers for Disease Control and Prevention, Atlanta, GA USA)) or clinical specimens.

Bacterial capsular saccharides can be obtained directly from bacteria using isolation procedures known to one of ordinary skill in the art (see for example methods disclosed in US2006/0228380, US2006/0228381, US2007/0184071, US2007/0184072, US2007/0231340, and US2008/0102498 and WO2008/118752). They can also be produced using synthetic protocols known to the man skilled in the art.

In case the bacterial capsular saccharide is obtained directly from bacteria, the bacterial cells can be grown in a medium. Following fermentation of bacterial cells that produce the capsular saccharide, the bacterial cells can be lysed to produce a cell lysate. The capsular saccharide may then be isolated from the cell lysate using purification techniques known in the art, including the use of centrifugation, depth filtration, precipitation, ultra-filtration, treatment with activate carbon, diafiltration and/or column chromatography (see, for example, US2006/0228380, US2006/0228381 and WO2008/118752). The purified capsular saccharide can then be used for the preparation of immunogenic conjugates.

The isolated capsular saccharide obtained by purification can be characterized by different parameters including, for example the weight average molecular weight (Mw). The molecular weight of the polysaccharide can be measured by Size Exclusion Chromatography (SEC) combined with Multiangle Laser Light Scattering detector (MALLS).

In an embodiment, the capsular saccharide used in the method of making or part of the glycoconjugate of the present invention is a capsular saccharide from a pathogenic bacteria. Preferably, the capsular saccharide used in the present invention is a capsular saccharide from a pathogenic Streptococcus, a pathogenic Staphylococcus, a pathogenic Enterococcus, a pathogenic Bacillus, a pathogenic Corynebacterium, a pathogenic Listeria, a pathogenic Erysipelothrix, a pathogenic Clostridium, a pathogenic Haemophilus, a pathogenic Neisseria or a pathogenic Escherichia. More preferably, the capsular saccharide used in the present invention is a capsular saccharide from a pathogenic Streptococcus, a pathogenic Neisseria or a pathogenic Escherichia.

In an embodiment, the capsular saccharide used in the present invention is a capsular saccharide from Aeromonas hydrophila and other species (spp.); Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Chlamydia trachomatis, Clostridium botulinum; Clostridium dificile; Clostridium perfringens; Coccidioides immitis; Coccidioides posadasii; Cowdria ruminantium (Heartwater); Coxiella burnetii; Enterococcus faecalis; Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli—enteroinvasive (EIEC); Ehrlichia spp. such as Ehrlichia chajfeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Peronosclerosporaphilippinensis; Phakopsora pachyrhizi; Plesiomonas shigelloides; Ralstonia solanacearum race 3, biovar 2; Rickettsia prowazekii; Rickettsia rickettsii; Salmonella spp.; Schlerophthora rayssiae var zeae; Shigella spp.; Staphylococcus aureus; Streptococcus; Synchytrium endobioticum; Vibrio cholerae non-01; Vibrio cholerae 01; Vibrio par ahaemo ly ticus and other Vibrios; Vibrio vulnificus; Xanthomonas oryzae; Xylella fastidiosa (citrus variegated chlorosis strain); Yersinia enterocolitica and Yersinia pseudotuberculosis; or Yersinia pestis. Preferably, the capsular saccharide used in the present invention is a capsular saccharide from Enterococcus faecalis, Escherichia coli, Staphylococcus aureus or Streptococcus.

In an embodiment, the capsular saccharide used in the present invention is a capsular saccharide from Haemophilus influenzae, Neisseria meningitidis, S. pneumoniae, S. pyogenes, S. agalactiae, Group C & G Streptococci or Escherichia coli.

More preferably, the capsular saccharide used in the present invention is a capsular saccharide from Neisseria meningitidis, S. pneumoniae, S. agalactiae or Escherichia coli. Even more preferably, the capsular saccharide used in the present invention is a capsular saccharide from S. pneumoniae or S. agalactiae. Even more preferably, the capsular saccharide used in the present invention is a capsular saccharide from S. pneumoniae.

In an embodiment, the capsular saccharide used in the present invention is a capsular saccharide from Staphylococcus aureus. In an embodiment, the capsular saccharide used in the present invention is a capsular saccharide from Staphylococcus aureus type 5 or Staphylococcus aureus type 8.

In an embodiment, the capsular saccharide used in the present invention is is a capsular saccharide from Enterococcus faecalis. In yet a further embodiment, the capsular saccharide used in the present invention is a capsular saccharide from is Haemophilus influenzae type b.

In a further embodiment, the capsular saccharide used in the present invention is a capsular saccharide from Neisseria meningitidis. In an embodiment the capsular saccharide used in the present invention is a capsular saccharide from N. meningitidis serogroup A (MenA), N. meningitidis serogroup W135 (MenW135), N. meningitidis serogroup Y (MenY), N. meningitidis serogroup X (MenX) or N. meningitidis serogroup C (MenC). In an embodiment the capsular saccharide used in the present invention is a capsular saccharide from N. meningitidis serogroup A (MenA). In an embodiment the capsular saccharide used in the present invention is a capsular saccharide from N. meningitidis serogroup W135 (MenW135). In an embodiment the capsular saccharide used in the present invention is a capsular saccharide from N. meningitidis serogroup Y (MenY). In an embodiment the capsular saccharide used in the present invention is a capsular saccharide from N. meningitidis serogroup C (MenC). In an embodiment the capsular saccharide used in the present invention is a capsular saccharide from N. meningitidis serogroup X (MenX).

In a further embodiment, the capsular saccharide used in the present invention is a capsular saccharide from Escherichia coli. In a further embodiment, the capsular saccharide used in the present invention is a capsular saccharide from Enterococcus faecalis.

In a further embodiment, the capsular saccharide used in the present invention is a capsular saccharide from Streptococcus agalactiae (Group B streptococcus (GBS)). In some embodiments, the capsular saccharide used in the present invention is a capsular saccharide from GBS type Ia, Ib, II, III, IV, V, VI, VII or VIII. In some embodiments, the capsular saccharide used in the present invention is a capsular saccharide from GBS types Ia, Ib, II, III or V.

In a further embodiment, the capsular saccharide used in the present invention is a capsular saccharide from Escherichia coli. In an embodiment, the capsular saccharide used in the present invention is a capsular saccharide from an Escherichia coli part of the Enterovirulent Escherichia coli group (EEC Group) such as Escherichia coli—enterotoxigenic (ETEC), Escherichia coli—enteropathogenic (EPEC), Escherichia coli—O157:H7 enterohemorrhagic (EHEC), or Escherichia coli—enteroinvasive (EIEC). In an embodiment, the capsular saccharide used in the present invention is a capsular saccharide from an Uropathogenic Escherichia coli (UPEC).

In an embodiment, the capsular saccharide used in the present invention is a capsular saccharide from an Escherichia coli serotype selected from the group consisting of serotypes O157:H7, O26:H11, O111:H— and O103:H2. In an embodiment, the capsular saccharide used in the present invention is a capsular saccharide from an Escherichia coli serotype selected from the group consisting of serotypes O6:K2:H1 and O18:K1:H7.

In an embodiment, the capsular saccharide used in the present invention is a capsular saccharide from an Escherichia coli serotype selected from the group consisting of serotypes O45:K1, O17:K52:H18, O19:H34 and O7:K1. In an embodiment, the capsular saccharide used in the present invention is a capsular saccharide from an Escherichia coli serotype O104:H4. In an embodiment, the capsular saccharide used in the present invention is a capsular saccharide from an Escherichia coli serotype O1:K12:H7. In an embodiment, the capsular saccharide used in the present invention is a capsular saccharide from an Escherichia coli serotype O127:H6. In an embodiment, the capsular saccharide used in the present invention is a capsular saccharide from an Escherichia coli serotype O139:H28. In an embodiment, the capsular saccharide used in the present invention is a capsular saccharide from an Escherichia coli serotype O128:H2.

In a preferred embodiment, the capsular saccharide used in the present invention is a capsular saccharide from Steptococcus pneumoniae. Preferably, the capsular saccharide used in the present invention is a capsular saccharide from a Streptococcus pneumoniae serotype selected from the group consisting of serotypes 1, 2, 4, 5, 6A, 6B, 6C, 7C, 7F, 8, 9V, 9N, 10A, 10B, 11A, 12F, 14, 15A, 15B, 15C, 16F, 17F, 18C, 19A, 19F, 20, 21, 22A, 22F, 23A, 23B, 23F, 24B, 24F, 27, 29, 31, 33B, 33F, 34, 35B, 35F, 38, 72 and 73.

In a preferred embodiment, the capsular saccharide used in the present invention is not a capsular saccharide from Streptococcus pneumoniae serotype 3.

In an embodiment, the capsular saccharide used in the present invention is a capsular saccharide from Streptococcus pneumoniae serotype 1. In an embodiment, the capsular saccharide used in the present invention is a capsular saccharide from Streptococcus pneumoniae serotype 2. In an embodiment, the capsular saccharide used in the present invention is a capsular saccharide from Streptococcus pneumoniae serotype 4. In an embodiment, the capsular saccharide used in the present invention is a capsular saccharide from Streptococcus pneumoniae serotype 5. In an embodiment, the capsular saccharide used in the present invention is a capsular saccharide from Streptococcus pneumoniae serotype 6A. In an embodiment, the capsular saccharide used in the present invention is a capsular saccharide from Streptococcus pneumoniae serotype 6B. In an embodiment, the capsular saccharide used in the present invention is a capsular saccharide from Streptococcus pneumoniae serotype 7C. In an embodiment, the capsular saccharide used in the present invention is a capsular saccharide from Streptococcus pneumoniae serotype 7F. In an embodiment, the capsular saccharide used in the present invention is a capsular saccharide from Streptococcus pneumoniae serotype 8. In an embodiment, the capsular saccharide used in the present invention is a capsular saccharide from Streptococcus pneumoniae serotype 9V. In an embodiment, the capsular saccharide used in the present invention is a capsular saccharide from Streptococcus pneumoniae serotype 9N. In an embodiment, the capsular saccharide used in the present invention is a capsular saccharide from Streptococcus pneumoniae serotype 10A. In an embodiment, the capsular saccharide used in the present invention is a capsular saccharide from Streptococcus pneumoniae serotype 10B. In an embodiment, the capsular saccharide used in the present invention is a capsular saccharide from Streptococcus pneumoniae serotype 11A. In an embodiment, the capsular saccharide used in the present invention is a capsular saccharide from Streptococcus pneumoniae serotype 12F. In an embodiment, the capsular saccharide used in the present invention is a capsular saccharide from Streptococcus pneumoniae serotype 14. In an embodiment, the capsular saccharide used in the present invention is a capsular saccharide from Streptococcus pneumoniae serotype 15A. In an embodiment, the capsular saccharide used in the present invention is a capsular saccharide from Streptococcus pneumoniae serotype 15B. In an embodiment, the capsular saccharide used in the present invention is a capsular saccharide from Streptococcus pneumoniae serotype 15C. In an embodiment, the capsular saccharide used in the present invention is a capsular saccharide from Streptococcus pneumoniae serotype 16F. In an embodiment, the capsular saccharide used in the present invention is a capsular saccharide from Streptococcus pneumoniae serotype 17F. In an embodiment, the capsular saccharide used in the present invention is a capsular saccharide from Streptococcus pneumoniae serotype 18C. In an embodiment, the capsular saccharide used in the present invention is a capsular saccharide from Streptococcus pneumoniae serotype 19A. In an embodiment, the capsular saccharide used in the present invention is a capsular saccharide from Streptococcus pneumoniae serotype 19F. In an embodiment, the capsular saccharide used in the present invention is a capsular saccharide from Streptococcus pneumoniae serotype 20. In an embodiment, the capsular saccharide used in the present invention is a capsular saccharide from Streptococcus pneumoniae serotype 21. In an embodiment, the capsular saccharide used in the present invention is a capsular saccharide from Streptococcus pneumoniae serotype 22A. In an embodiment, the capsular saccharide used in the present invention is a capsular saccharide from Streptococcus pneumoniae serotype 22F. In an embodiment, the capsular saccharide used in the present invention is a capsular saccharide from Streptococcus pneumoniae serotype 23A. In an embodiment, the capsular saccharide used in the present invention is a capsular saccharide from Streptococcus pneumoniae serotype 23B. In an embodiment, the capsular saccharide used in the present invention is a capsular saccharide from Streptococcus pneumoniae serotype 23F. In an embodiment, the capsular saccharide used in the present invention is a capsular saccharide from Streptococcus pneumoniae serotype 24B. In an embodiment, the capsular saccharide used in the present invention is a capsular saccharide from Streptococcus pneumoniae serotype 24F. In an embodiment, the capsular saccharide used in the present invention is a capsular saccharide from Streptococcus pneumoniae serotype 27. In an embodiment, the capsular saccharide used in the present invention is a capsular saccharide from Streptococcus pneumoniae serotype 29. In an embodiment, the capsular saccharide used in the present invention is a capsular saccharide from Streptococcus pneumoniae serotype 31. In an embodiment, the capsular saccharide used in the present invention is a capsular saccharide from Streptococcus pneumoniae serotype 33B. In an embodiment, the capsular saccharide used in the present invention is a capsular saccharide from Streptococcus pneumoniae serotype 33F. In an embodiment, the capsular saccharide used in the present invention is a capsular saccharide from Streptococcus pneumoniae serotype 34. In an embodiment, the capsular saccharide used in the present invention is a capsular saccharide from Streptococcus pneumoniae serotype 35B. In an embodiment, the capsular saccharide used in the present invention is a capsular saccharide from Streptococcus pneumoniae serotype 35F. In an embodiment, the capsular saccharide used in the present invention is a capsular saccharide from Streptococcus pneumoniae serotype 38. In an embodiment, the capsular saccharide used in the present invention is a capsular saccharide from Streptococcus pneumoniae serotype 72. In an embodiment, the capsular saccharide used in the present invention is a capsular saccharide from Streptococcus pneumoniae serotype 73.

In a preferred embodiment, the capsular saccharide used in the present invention is a capsular saccharide from Streptococcus pneumoniae serotype 35B. In another preferred embodiment, the capsular saccharide used in the present invention is a capsular saccharide from Streptococcus pneumoniae serotype 29.

In a preferred embodiment, the capsular saccharide used in the present invention (purified before further treatment) has a weight average molecular weight between 50 kDa and 5000 kDa. In a preferred embodiment, the capsular saccharide used in the present invention has a weight average molecular weight between 500 kDa and 5000 kDa. In another preferred embodiment, the capsular saccharide used in the present invention has a weight average molecular weight between 1000 kDa and 5000 kDa.

Any whole number integer within any of the above ranges is contemplated as an embodiment of the disclosure.

Preferably, in order to generate glycoconjugates with advantageous filterability characteristics, immunogenicity and/or yields, sizing of the saccharide to a target molecular weight range is performed prior to the conjugation to a carrier protein.

Advantageously, the size of the purified capsular saccharide is reduced while preserving critical features of the structure of the polysaccharide. Mechanical or chemical sizing maybe employed.

In an embodiment, the size of the purified capsular saccharide is reduced by chemical hydrolysis. Chemical hydrolysis maybe conducted using a mild acid (e.g acetic acid, formic acid, propanoic acid). In an embodiment, chemical hydrolysis is conducted using formic acid. In an embodiment, chemical hydrolysis is conducted using propanoic acid. In a preferred embodiment, chemical hydrolysis is conducted using acetic acid. Chemical hydrolysis may also be conducted using a diluted strong acid (such as diluted hydrochloric acid, diluted sulfuric acid, diluted phosphoric acid, diluted nitric acid or diluted perchloric acid). In an embodiment, chemical hydrolysis is conducted using diluted hydrochloric acid. In an embodiment, chemical hydrolysis is conducted using diluted sulfuric acid. In an embodiment, chemical hydrolysis is conducted using diluted phosphoric acid. In an embodiment, chemical hydrolysis is conducted using diluted nitric acid. In an embodiment, chemical hydrolysis is conducted using diluted perchloric acid.

The size of the purified capsular saccharide can also be reduced by mechanical homogenization. In an embodiment, the size of the purified capsular saccharide is reduced by high pressure homogenization. High pressure homogenization achieves high shear rates by pumping the process stream through a flow path with sufficiently small dimensions. The shear rate is increased by using a larger applied homogenization pressure, and exposure time can be increased by recirculating the feed stream through the homogenizer.

The high-pressure homogenization process can be appropriate for reducing the size of the purified capsular saccharide while preserving the structural features of the saccharide.

In a preferred embodiment, the isolated capsular saccharide is sized to a weight average molecular weight between 10 kDa and 1000 kDa. In an embodiment, the isolated capsular saccharide is sized to a weight average molecular weight between 50 kDa and 500 kDa. In an embodiment, the isolated capsular saccharide is sized to a weight average molecular weight between 50 kDa and 400 kDa. In an embodiment, the isolated capsular saccharide is sized to a weight average molecular weight between 50 kDa and 250 kDa.

In an embodiment, the isolated capsular saccharide is sized to a weight average molecular weight between 250 kDa and 1000 kDa. In an embodiment, the isolated capsular saccharide is sized to a weight average molecular weight between 250 kDa and 500 kDa. In an embodiment, the isolated capsular saccharide is sized to a weight average molecular weight between 250 kDa and 400 kDa. In a preferred embodiment, the isolated capsular saccharide is sized to a weight average molecular weight between 200 kDa and 800 kDa.

In an embodiment, the isolated capsular saccharide is sized to a weight average molecular weight of about 250 kDa. In an embodiment, the isolated capsular saccharide is sized to a weight average molecular weight of about 300 kDa. In an embodiment, the isolated capsular saccharide is sized to a weight average molecular weight of about 350 kDa. In an embodiment, the isolated capsular saccharide is sized to a weight average molecular weight of about 400 kDa. In an embodiment, the isolated capsular saccharide is sized to a weight average molecular weight of about 450 kDa. In an embodiment, the isolated capsular saccharide is sized to a weight average molecular weight of about 500 kDa. In an embodiment, the isolated capsular saccharide is sized to a weight average molecular weight of about 550 kDa. In an embodiment, the isolated capsular saccharide is sized to a weight average molecular weight of about 600 kDa. In an embodiment, the isolated capsular saccharide is sized to a weight average molecular weight of about 700 kDa. In an embodiment, the isolated capsular saccharide is sized to a weight average molecular weight of about 800 kDa. In an embodiment, the isolated capsular saccharide is sized to a weight average molecular weight of about 900 kDa. In an embodiment, the isolated capsular saccharide is sized to a weight average molecular weight of about 1000 kDa.

In an embodiment, the isolated capsular saccharide is not sized.

The isolated capsular saccharide described above may be activated (e.g., chemically activated) to make them capable of reacting (e.g. with a linker) and then incorporated into glycoconjugates, as further described herein.

For the purposes of the invention the term ‘glycoconjugate’ indicates a saccharide covalently linked to a carrier protein.

In general, covalent conjugation of saccharides to carriers enhances the immunogenicity of saccharides as it converts them from T-independent antigens to T-dependent antigens, thus allowing priming for immunological memory. Conjugation is particularly useful for pediatric vaccines.

### 1.2 Capsular Saccharide Glycoconjugates of the Invention

In some embodiments, the glycoconjugate of the present invention comprises a capsular saccharide wherein the weight average molecular weight (Mw) of said polysaccharide before conjugation is between 10 kDa and 2,000 kDa.

The weight average molecular weight (Mw) of the saccharide before conjugation refers to the Mw before the activation of the saccharide (i.e. after an eventual sizing step but before reacting the saccharide with an activating agent). In the context of the present invention the Mw of the saccharide is not substantially modified by the activation step and the Mw of the saccharide incorporated in the conjugate is similar to the Mw of the saccharide as measured before activation. In an embodiment, the saccharide is activated with a carbonic acid derivative (e.g. CDI or CDT) in combination with an azido linker (see sections 1.3 below). In an embodiment, the saccharide is activated with CDI in combination with an azido linker (see sections 1.3 below). In an embodiment, the saccharide is activated with CDT in combination with an azido linker (see sections 1.3 below).

In an embodiment, the glycoconjugate of the present invention comprises a capsular saccharide wherein the weight average molecular weight (Mw) of said saccharide before conjugation is between 50 kDa and 1,000 kDa. In an embodiment, the weight average molecular weight (Mw) is between 50 kDa and 750 kDa. In an embodiment, the weight average molecular weight (Mw) is between 50 kDa and 500 kDa.

In an embodiment, the weight average molecular weight (Mw) is between 50 kDa and 250 kDa. In an embodiment, the weight average molecular weight (Mw) is between 50 kDa and 200 kDa. In an embodiment, the weight average molecular weight (Mw) is between 50 kDa and 150 kDa. In an embodiment, the weight average molecular weight (Mw) is between 50 kDa and 100 kDa.

In an embodiment, the glycoconjugate of the present invention comprises a capsular saccharide wherein the weight average molecular weight (Mw) of said saccharide before conjugation is between 75 kDa and 1,000 kDa. In an embodiment, the weight average molecular weight (Mw) is between 75 kDa and 750 kDa. In an embodiment, the weight average molecular weight (Mw) is between 75 kDa and 500 kDa.

In an embodiment, the weight average molecular weight (Mw) is between 75 kDa and 250 kDa. In an embodiment, the weight average molecular weight (Mw) is between 75 kDa and 200 kDa. In an embodiment, the weight average molecular weight (Mw) is between 75 kDa and 150 kDa. In an embodiment, the weight average molecular weight (Mw) is between 75 kDa and 100 kDa.

In an embodiment, the glycoconjugate of the present invention comprises a capsular saccharide wherein the weight average molecular weight (Mw) of said saccharide before conjugation is between 100 kDa and 1,000 kDa. In an embodiment, the weight average molecular weight (Mw) is between 100 kDa and 750 kDa. In an embodiment, the weight average molecular weight (Mw) is between 100 kDa and 500 kDa. In an embodiment, the weight average molecular weight (Mw) is between 100 kDa and 250 kDa. In an embodiment, the weight average molecular weight (Mw) is between 100 kDa and 200 kDa. In an embodiment, the weight average molecular weight (Mw) is between 100 kDa and 150 kDa.

In an embodiment, the glycoconjugate of the present invention comprises a capsular saccharide wherein the weight average molecular weight (Mw) of said saccharide before conjugation is between 150 kDa and 1,000 kDa. In an embodiment, the weight average molecular weight (Mw) is between 150 kDa and 750 kDa. In an embodiment, the weight average molecular weight (Mw) is between 150 kDa and 500 kDa. In an embodiment, the weight average molecular weight (Mw) is between 150 kDa and 250 kDa. In an embodiment, the weight average molecular weight (Mw) is between 150 kDa and 200 kDa.

In an embodiment, the glycoconjugate of the present invention comprises a capsular saccharide wherein the weight average molecular weight (Mw) of said saccharide before conjugation is between 200 kDa and 1,000 kDa. In an embodiment, the weight average molecular weight (Mw) is between 200 kDa and 750 kDa. In an embodiment, the weight average molecular weight (Mw) is between 200 kDa and 500 kDa. In an embodiment, the weight average molecular weight (Mw) is between 200 kDa and 300 kDa. In an embodiment, the weight average molecular weight (Mw) is between 200 kDa and 250 kDa.

In an embodiment, the glycoconjugate of the present invention comprises a capsular saccharide wherein the weight average molecular weight (Mw) of said saccharide before conjugation is between 500 kDa and 1,000 kDa. In an embodiment, the weight average molecular weight (Mw) is between 500 kDa and 750 kDa. In an embodiment, the weight average molecular weight (Mw) is between 500 kDa and 700 kDa. In an embodiment, the weight average molecular weight (Mw) is between 500 kDa and 600 kDa.

Any whole number integer within any of the above ranges is contemplated as an embodiment of the disclosure.

In an embodiment, the glycoconjugate of the present invention comprises a capsular saccharide wherein the weight average molecular weight (Mw) of said saccharide before conjugation is about 1,000 kDa. In an embodiment, the weight average molecular weight (Mw) is about 750 kDa. In an embodiment, the weight average molecular weight (Mw) is about 700 kDa. In an embodiment, the weight average molecular weight (Mw) is about 600 kDa. In an embodiment, the weight average molecular weight (Mw) is about 500 kDa. In an embodiment, the weight average molecular weight (Mw) is about 400 kDa. In an embodiment, the weight average molecular weight (Mw) is about 300 kDa. In an embodiment, the weight average molecular weight (Mw) is about 200 kDa. In an embodiment, the weight average molecular weight (Mw) is about 150 kDa. In an embodiment, the weight average molecular weight (Mw) is about 125 kDa. In an embodiment, the weight average molecular weight (Mw) is about 100 kDa.

In some embodiments, the glycoconjugate of the invention has a weight average molecular weight (Mw) of between 250 kDa and 20,000 kDa. In other embodiments, the glycoconjugate of the invention has a weight average molecular weight (Mw) of between 500 kDa and 15,000 kDa. In yet other embodiments, the glycoconjugate of the invention has a weight average molecular weight (Mw) of between 500 kDa and 10,000 kDa.

In other embodiments, the glycoconjugate of the invention has a weight average molecular weight (Mw) of between 500 kDa and 10,000 kDa. In other embodiments, the glycoconjugate of the invention has a weight average molecular weight (Mw) of between 500 kDa and 7,500 kDa. In still other embodiments, the glycoconjugate of the invention has a weight average molecular weight (Mw) of between 500 kDa and 5,000 kDa. In still other embodiments, the glycoconjugate of the invention has a weight average molecular weight (Mw) of between 500 kDa and 2,500 kDa. In still other embodiments, the glycoconjugate of the invention has a weight average molecular weight (Mw) of between 500 kDa and 2,000 kDa. In still other embodiments, the glycoconjugate of the invention has a weight average molecular weight (Mw) of between 500 kDa and 1,500 kDa. In still other embodiments, the glycoconjugate of the invention has a weight average molecular weight (Mw) of between 500 kDa and 1,000 kDa. In still other embodiments, the glycoconjugate of the invention has a weight average molecular weight (Mw) of between 500 kDa and 750 kDa.

In other embodiments, the glycoconjugate of the invention has a weight average molecular weight (Mw) of between 750 kDa and 10,000 kDa. In other embodiments, the glycoconjugate of the invention has a weight average molecular weight (Mw) of between 750 kDa and 7,500 kDa. In still other embodiments, the glycoconjugate of the invention has a weight average molecular weight (Mw) of between 750 kDa and 5,000 kDa. In still other embodiments, the glycoconjugate of the invention has a weight average molecular weight (Mw) of between 750 kDa and 2,500 kDa. In still other embodiments, the glycoconjugate of the invention has a weight average molecular weight (Mw) of between 750 kDa and 2,000 kDa. In still other embodiments, the glycoconjugate of the invention has a weight average molecular weight (Mw) of between 750 kDa and 1,500 kDa. In still other embodiments, the glycoconjugate of the invention has a weight average molecular weight (Mw) of between 750 kDa and 1,000 kDa.

In other embodiments, the glycoconjugate of the invention has a weight average molecular weight (Mw) of between 1,000 kDa and 10,000 kDa. In other embodiments, the glycoconjugate of the invention has a weight average molecular weight (Mw) of between 1,000 kDa and 7,500 kDa. In still other embodiments, the glycoconjugate of the invention has a weight average molecular weight (Mw) of between 1,000 kDa and 5,000 kDa. In still other embodiments, the glycoconjugate of the invention has a weight average molecular weight (Mw) of between 1,000 kDa and 2,500 kDa. In still other embodiments, the glycoconjugate of the invention has a weight average molecular weight (Mw) of between 1,000 kDa and 2,000 kDa. In still other embodiments, the glycoconjugate of the invention has a weight average molecular weight (Mw) of between 1,000 kDa and 1,500 kDa.

In other embodiments, the glycoconjugate of the invention has a weight average molecular weight (Mw) of between 2,000 kDa and 10,000 kDa. In other embodiments, the glycoconjugate of the invention has a weight average molecular weight (Mw) of between 2,000 kDa and 7,500 kDa. In still other embodiments, the glycoconjugate of the invention has a weight average molecular weight (Mw) of between 2,000 kDa and 5,000 kDa. In still other embodiments, the glycoconjugate of the invention has a weight average molecular weight (Mw) of between 2,000 kDa and 4,000 kDa. In still other embodiments, the glycoconjugate of the invention has a weight average molecular weight (Mw) of between 2,000 kDa and 3,000 kDa. In still other embodiments, the glycoconjugate of the invention has a weight average molecular weight (Mw) of between 2,000 kDa and 3,500 kDa.

In still other embodiments, the glycoconjugate of the invention has a weight average molecular weight (Mw) of between 2,250 kDa and 3,500 kDa.

In preferred embodiment, the glycoconjugate of the invention has a weight average molecular weight (Mw) of between 1,000 kDa and 2,500 kDa.

Any whole number integer within any of the above ranges is contemplated as an embodiment of the disclosure.

In other embodiments, the glycoconjugate of the invention has a weight average molecular weight (Mw) of about 10,000 kDa. In other embodiments, the glycoconjugate of the invention has a weight average molecular weight (Mw) of about 9,000 kDa. In other embodiments, the glycoconjugate of the invention has a weight average molecular weight (Mw) of about 8,000 kDa. In still other embodiments, the glycoconjugate of the invention has a weight average molecular weight (Mw) of about 7,500 kDa. In still other embodiments, the glycoconjugate of the invention has a weight average molecular weight (Mw) of about 6,000 kDa. In still other embodiments, the glycoconjugate of the invention has a weight average molecular weight (Mw) of about 5,000 kDa. In still other embodiments, the glycoconjugate of the invention has a weight average molecular weight (Mw) of about 4,000 kDa. In still other embodiments, the glycoconjugate of the invention has a weight average molecular weight (Mw) of about 3,500 kDa. In still other embodiments, the glycoconjugate of the invention has a weight average molecular weight (Mw) of about 3,250 kDa. In still other embodiments, the glycoconjugate of the invention has a weight average molecular weight (Mw) of about 3,000 kDa. In still other embodiments, the glycoconjugate of the invention has a weight average molecular weight (Mw) of about 2,500 kDa. In still other embodiments, the glycoconjugate of the invention has a weight average molecular weight (Mw) of about 2,000 kDa. In still other embodiments, the glycoconjugate of the invention has a weight average molecular weight (Mw) of about 1,500 kDa. In still other embodiments, the glycoconjugate of the invention has a weight average molecular weight (Mw) of about 1,000 kDa. In still other embodiments, the glycoconjugate of the invention has a weight average molecular weight (Mw) of about 750 kDa.

Another way to characterize the glycoconjugates of the invention is by the number of lysine residues in the carrier protein (e.g., CRM₁₉₇ or SCP) that become conjugated to the saccharide which can be characterized as a range of conjugated lysines (degree of conjugation). The evidence for lysine modification of the carrier protein, due to covalent linkages to the saccharides, can be obtained by amino acid analysis using routine methods known to those of skill in the art. Conjugation results in a reduction in the number of lysine residues recovered compared to the carrier protein starting material used to generate the conjugate materials. In a preferred embodiment, the degree of conjugation of the glycoconjugate of the invention is between 2 and 15. In an embodiment, the degree of conjugation of the glycoconjugate of the invention is between 2 and 13. In an embodiment, the degree of conjugation of the glycoconjugate of the invention is between 2 and 10. In an embodiment, the degree of conjugation of the glycoconjugate of the invention is between 2 and 8. In an embodiment, the degree of conjugation of the glycoconjugate of the invention is between 2 and 6. In an embodiment, the degree of conjugation of the glycoconjugate of the invention is between 2 and 5. In an embodiment, the degree of conjugation of the glycoconjugate of the invention is between 2 and 4. In an embodiment, the degree of conjugation of the glycoconjugate of the invention is between 3 and 15. In an embodiment, the degree of conjugation of the glycoconjugate of the invention is between 3 and 13. In an embodiment, the degree of conjugation of the glycoconjugate of the invention is between 3 and 10. In an embodiment, the degree of conjugation of the glycoconjugate of the invention is between 3 and 8. In an embodiment, the degree of conjugation of the glycoconjugate of the invention is between 3 and 6. In an embodiment, the degree of conjugation of the glycoconjugate of the invention is between 3 and 5. In an embodiment, the degree of conjugation of the glycoconjugate of the invention is between 3 and 4. In an embodiment, the degree of conjugation of the glycoconjugate of the invention is between 5 and 15. In an embodiment, the degree of conjugation of the glycoconjugate of the invention is between 5 and 10. In an embodiment, the degree of conjugation of the glycoconjugate of the invention is between 8 and 15. In an embodiment, the degree of conjugation of the glycoconjugate of the invention is between 8 and 12. In an embodiment, the degree of conjugation of the glycoconjugate of the invention is between 10 and 15. In an embodiment, the degree of conjugation of the glycoconjugate of the invention is between 10 and 12.

In an embodiment, the degree of conjugation of the glycoconjugate of the invention is about 2. In an embodiment, the degree of conjugation of the glycoconjugate of the invention is about 3. In an embodiment, the degree of conjugation of the glycoconjugate of the invention is about 4. In an embodiment, the degree of conjugation of the glycoconjugate of the invention is about 5. In an embodiment, the degree of conjugation of the glycoconjugate of the invention is about 6. In an embodiment, the degree of conjugation of the glycoconjugate of the invention is about 7. In an embodiment, the degree of conjugation of the glycoconjugate of the invention is about 8. In an embodiment, the degree of conjugation of the glycoconjugate of the invention is about 9. In an embodiment, the degree of conjugation of the glycoconjugate of the invention is about 10, about 11. In an embodiment, the degree of conjugation of the glycoconjugate of the invention is about 12. In an embodiment, the degree of conjugation of the glycoconjugate of the invention is about 13. In an embodiment, the degree of conjugation of the glycoconjugate of the invention is about 14. In an embodiment, the degree of conjugation of the glycoconjugate of the invention is about 15. In a preferred embodiment, the degree of conjugation of the glycoconjugate of the invention is between 4 and 7. In some such embodiments, the carrier protein is CRM₁₉₇. In other such embodiments, the carrier protein is SCP.

The glycoconjugates of the invention may also be characterized by the ratio (weight/weight) of saccharide to carrier protein. In some embodiments, the ratio of saccharide to carrier protein in the glycoconjugate (w/w) is between 0.5 and 3.0. In other embodiments, the saccharide to carrier protein ratio (w/w) is between 0.5 and 2.0. In other embodiments, the saccharide to carrier protein ratio (w/w) is between 0.5 and 1.5. In other embodiments, the saccharide to carrier protein ratio (w/w) is between 0.8 and 1.2. In other embodiments, the saccharide to carrier protein ratio (w/w) is between 0.5 and 1.0. In other embodiments, the saccharide to carrier protein ratio (w/w) is between 1.0 and 1.5. In other embodiments, the saccharide to carrier protein ratio (w/w) is between 1.0 and 2.0. In further embodiments, the saccharide to carrier protein ratio (w/w) is between 0.8 and 1.2. In a preferred embodiment, the ratio of saccharide to carrier protein in the conjugate is between 0.9 and 1.1.

In an embodiment, the saccharide to carrier protein ratio (w/w) is about 0.5. In other embodiments, the saccharide to carrier protein ratio (w/w) is about 0.6. In other embodiments, the saccharide to carrier protein ratio (w/w) is about 0.7. In other embodiments, the saccharide to carrier protein ratio (w/w) is about 0.8. In other embodiments, the saccharide to carrier protein ratio (w/w) is about 0.9. In other embodiments, the saccharide to carrier protein ratio (w/w) is about 1.0. In other embodiments, the saccharide to carrier protein ratio (w/w) is about 1.1. In other embodiments, the saccharide to carrier protein ratio (w/w) is about 1.2. In other embodiments, the saccharide to carrier protein ratio (w/w) is about 1.3. In other embodiments, the saccharide to carrier protein ratio (w/w) is about 1.4. In other embodiments, the saccharide to carrier protein ratio (w/w) is about 1.5. In other embodiments, the saccharide to carrier protein ratio (w/w) is about 1.6. In other embodiments, the saccharide to carrier protein ratio (w/w) is about 1.7. In other embodiments, the saccharide to carrier protein ratio (w/w) is about 1.8. In other embodiments, the saccharide to carrier protein ratio (w/w) is about 1.9. In other embodiments, the saccharide to carrier protein ratio (w/w) is about 2.0. In other embodiments, the saccharide to carrier protein ratio (w/w) is about 2.1. In other embodiments, the saccharide to carrier protein ratio (w/w) is about 2.2. In other embodiments, the saccharide to carrier protein ratio (w/w) is about 2.5. In other embodiments, the saccharide to carrier protein ratio (w/w) is about 2.8. In other embodiments, the saccharide to carrier protein ratio (w/w) is about 3.0. In some such embodiments, the carrier protein is CRM₁₉₇. In other such embodiments, the carrier protein is SCP.

The glycoconjugates of the invention may also be characterized by the number of covalent linkages between the carrier protein and the saccharide as a function of repeat units of the saccharide. In one embodiment, the glycoconjugate of the invention comprises at least one covalent linkage between the carrier protein and the saccharide for every 4 saccharide repeat units of the saccharide. In another embodiment, the covalent linkage between the carrier protein and the saccharide occurs at least once in every 10 saccharide repeat units of the saccharide. In another embodiment, the covalent linkage between the carrier protein and the saccharide occurs at least once in every 15 saccharide repeat units of the saccharide. In a further embodiment, the covalent linkage between the carrier protein and the saccharide occurs at least once in every 25 saccharide repeat units of the saccharide. In a further embodiment, the covalent linkage between the carrier protein and the saccharide occurs at least once in every 50 saccharide repeat units of the saccharide. In yet a further embodiment, the covalent linkage between the carrier protein and the saccharide occurs at least once in every 100 saccharide repeat units of the saccharide.

In other embodiments, the glycoconjugate of the invention comprises at least one covalent linkage between the carrier protein and the saccharide for every 5 to 10 saccharide repeat units of the saccharide.

In other embodiments, the glycoconjugate of the invention comprises at least one covalent linkage between the carrier protein and the saccharide for every 2 to 7 saccharide repeat units of the saccharide.

In other embodiments, the glycoconjugate of the invention comprises at least one covalent linkage between the carrier protein and the saccharide for every 6 to 11 saccharide repeat units of the saccharide.

In other embodiments, the glycoconjugate of the invention comprises at least one covalent linkage between the carrier protein and the saccharide for every 9 to 14 saccharide repeat units of the saccharide.

In other embodiments, the glycoconjugate of the invention comprises at least one covalent linkage between the carrier protein and the saccharide for every 10 to 20 saccharide repeat units of the saccharide.

In other embodiments, the glycoconjugate of the invention comprises at least one covalent linkage between the carrier protein and the saccharide for every 4 to 25 saccharide repeat units of the saccharide.

In frequent embodiments, the carrier protein is CRM₁₉₇. In other embodiments, the carrier protein is SCP.

In some embodiments, the carrier protein is CRM₁₉₇ and the covalent linkage between the CRM₁₉₇ and the saccharide occurs at least once in every 4, 10, 15 or 25 saccharide repeat units of the saccharide. In other embodiments, the carrier protein is SCP and the covalent linkage between the SCP and the saccharide occurs at least once in every 4, 10, 15 or 25 saccharide repeat units of the saccharide.

The glycoconjugates and immunogenic compositions of the invention may contain free saccharide that is not covalently conjugated to the carrier protein but is nevertheless present in the glycoconjugate composition. The free saccharide may be noncovalently associated with (i.e., noncovalently bound to, adsorbed to, or entrapped in or with) the glycoconjugate.

In a preferred embodiment, the glycoconjugate comprises less than about 50% of free saccharide compared to the total amount of said saccharide. In a preferred embodiment the glycoconjugate comprises less than about 40% of free saccharide compared to the total amount of said saccharide. In a yet preferred embodiment, the glycoconjugate comprises less than about 25% of free saccharide compared to the total amount of said saccharide. In an even preferred embodiment, the glycoconjugate comprises less than about 20% of free saccharide compared to the total amount of said saccharide. In a yet preferred embodiment, the glycoconjugate comprises less than about 15% of free saccharide compared to the total amount of said saccharide.

The glycoconjugates may also be characterized by their molecular size distribution (K_{d}). Size exclusion chromatography media (CL-4B) can be used to determine the relative molecular size distribution of the conjugate. Size Exclusion Chromatography (SEC) is used in gravity fed columns to profile the molecular size distribution of conjugates. Large molecules excluded from the pores in the media elute more quickly than small molecules.

Fraction collectors are used to collect the column eluate. The fractions are tested colorimetrically by saccharide assay. For the determination of K_{d}, columns are calibrated to establish the fraction at which molecules are fully excluded (V₀), (K_{d}=0), and the fraction representing the maximum retention (V₁), (K_{d}=1). The fraction at which a specified sample attribute is reached (Vₑ), is related to K_{d} by the expression, K_{d}=(Vₑ−V₀)/(V₁−V₀).

In a preferred embodiment, at least 30% of the glycoconjugate of the invention has a K_{d} below or equal to 0.3 in a CL-4B column. In a preferred embodiment, at least 40% of the glycoconjugate of the invention has a K_{d} below or equal to 0.3 in a CL-4B column.

In a preferred embodiment, at least 45%, 50%, 55%, 60%, 65%, 70%, 75%, 80%, or 85% of the glycoconjugate of the invention has a K_{d} below or equal to 0.3 in a CL-4B column. In a preferred embodiment, at least 60% of the glycoconjugate of the invention has a K_{d} below or equal to 0.3 in a CL-4B column. In a preferred embodiment, between 50% and 80% of the glycoconjugate of the invention has a K_{d} below or equal to 0.3 in a CL-4B column. In a preferred embodiment, between 65% and 80% of the glycoconjugate of the invention has a K_{d} below or equal to 0.3 in a CL-4B column.

### 1.3 Capsular Saccharide Glycoconjugates of the Invention Prepared Using Click Chemistry

The glycoconjugates of the present invention are prepared using click chemistry. The invention also relates to a method of making a glycoconjugate, as disclosed herein.

According to the present invention, click chemistry comprises three steps, (a) reacting an isolated capsular saccharide with a carbonic acid derivative and an azido linker in an aprotic solvent to produce an activated azido saccharide (activation of the saccharide), (b) reacting a carrier protein with an agent bearing an N-Hydroxysuccinimide (NHS) moiety and an alkyne group where the NHS moiety reacts with the amino groups to form an amide linkage thereby obtaining an alkyne functionalized carrier protein (activation of the carrier protein), (c) reacting the activated azido saccharide of step (a) with the activated alkyne-carrier protein of step (b) by Cu⁺¹ mediated azide-alkyne cycloaddition reaction to form a glycoconjugate.

Following step (a) the saccharide is said to be activated and is referred to herein as “activated saccharide” or “activated azido saccharide”.

Following step (b) the carrier is said to be activated and is referred to as “activated carrier”.

As mentioned above, before the activation (a), sizing of the saccharide to a target molecular weight (MW) range can be performed. Therefore, in an embodiment, the isolated saccharide is sized before activation with a carbonic acid derivative and an azido linker. In an embodiment, the isolated saccharide is sized to any of the target molecular weight (MW) range defined above.

In an embodiment, said carbonic acid derivative is selected from the group consisting of 1,1′-carbonyldiimidazole (CDI), 1,1′-carbonyl-di-(1,2,4-triazole) (CDT), disuccinimidyl carbonate (DSC) and N-hydroxysuccinimidyl chloroformate.

In an embodiment, said carbonic acid derivative is 1,1′-carbonyldiimidazole (CDI). In another embodiment, said carbonic acid derivative is 1,1′-Carbonyl-di-(1,2,4-triazole) (CDT). In another embodiment, said carbonic acid derivative is disuccinimidyl carbonate (DSC). In yet a further embodiment, said carbonic acid derivative is N-hydroxysuccinimidyl chloroformate.

In an embodiment, said carbonic acid derivative is 1,1′-carbonyldiimidazole (CDI) or 1,1′-Carbonyl-di-(1,2,4-triazole) (CDT). Preferably, said carbonic acid derivative is 1,1′-carbonyldiimidazole (CDI).

In an embodiment, said azido linker is a compound of formula (I),

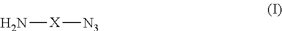

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.

In an embodiment, said azido linker is a compound of formula (I), wherein X is CH₂(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said azido linker is a compound of formula (I), wherein X is (CH₂CH₂O)ₘCH₂CH₂, wherein m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said azido linker is a compound of formula (I), wherein X is NHCO(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said azido linker is a compound of formula (I), wherein X is NHCO(CH₂CH₂O)ₘCH₂CH₂, where m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said azido linker is a compound of formula (I), wherein X is OCH₂(CH₂)ₙ, and n is selected from 1 to 10. In an embodiment, n is selected from 1 to 5. In an embodiment, n is selected from 1 to 4. In an embodiment, n is selected from 1 to 3. In an embodiment, n is selected from 1 to 2. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said azido linker is a compound of formula (I), wherein X is O(CH₂CH₂O)ₘCH₂CH₂, where m is selected from 1 to 4. In an embodiment, m is selected from 1 to 3. In an embodiment, m is selected from 1 to 2. In a particular embodiment, m is 1. In another embodiment, m is 2. In yet another embodiment, m is 3. In yet a further embodiment, m is 4.

In an embodiment, said azido linker is a compound of formula (II),

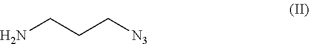

In a preferred embodiment, said azido linker is 3-azido-propylamine.

In an embodiment, said agent bearing an N-Hydroxysuccinimide (NHS) moiety and an alkyne group is an agent bearing an N-Hydroxysuccinimide (NHS) moiety and a terminal alkyne.

In an embodiment, said agent bearing an N-Hydroxysuccinimide (NHS) moiety and an alkyne group is an agent bearing an N-Hydroxysuccinimide (NHS) moiety and a cycloalkyne.

In an embodiment, said agent bearing an N-Hydroxysuccinimide (NHS) moiety and an alkyne group is a compound of formula (III),

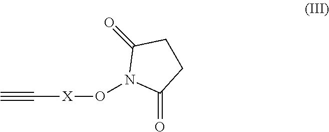

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

In an embodiment, said agent bearing an N-Hydroxysuccinimide (NHS) moiety and an alkyne group is a compound of formula (III), wherein X is CH₂O(CH₂)ₙCH₂C═O, where n is selected from 0 to 10. In an embodiment, n is selected from 0 to 5. In an embodiment, n is selected from 0 to 4. In an embodiment, n is selected from 0 to 3. In an embodiment, n is selected from 0 to 2. In a particular embodiment, n is 0. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10.

In an embodiment, said agent bearing an N-Hydroxysuccinimide (NHS) moiety and an alkyne group is a compound of formula (III), wherein X is CH₂O(CH₂CH₂O)ₘ(CH₂)ₙCH₂C═O, where n is selected from 0 to 10 and m is selected from 0 to 4. In an embodiment, n is selected from 0 to 5. In an embodiment, n is selected from 0 to 4. In an embodiment, n is selected from 0 to 3. In an embodiment, n is selected from 0 to 2. In a particular embodiment, n is 0. In a particular embodiment, n is 1. In another embodiment, n is 2. In yet another embodiment, n is 3. In yet a further embodiment, n is 4. In yet a further embodiment, n is 5. In yet a further embodiment, n is 6. In yet a further embodiment, n is 7. In yet a further embodiment, n is 8. In yet a further embodiment, n is 9. In yet a further embodiment, n is 10. In an embodiment, m is selected from 0 to 3. In an embodiment, m is selected from 0 to 2. In a particular embodiment, m is 1. In a particular embodiment, m is 1. In another embodiment, m is 2. In yet another embodiment, m is 3. In yet a further embodiment, m is 4.

In an embodiment, n is selected from 0 to 5 and m is selected from 0 to 3. In an embodiment, n is selected from 0 to 5 and m is selected from 0 to 2.

In an embodiment, n is selected from 0 to 4 and m is selected from 0 to 3. In an embodiment, n is selected from 0 to 4 and m is selected from 0 to 2.

In an embodiment, n is selected from 0 to 3 and m is selected from 0 to 3. In an embodiment, n is selected from 0 to 3 and m is selected from 0 to 2.

In an embodiment, n is selected from 0 to 2 and m is selected from 0 to 3. In an embodiment, n is selected from 0 to 2 and m is selected from 0 to 2.

In an embodiment, n is selected from 0 to 1 and m is selected from 0 to 3. In an embodiment, n is selected from 0 to 1 and m is selected from 0 to 2.

In an embodiment, n is 0 and m is 0. In an embodiment, n is 1 and m is 0. In an embodiment, n is 2 and m is 0. In an embodiment, n is 3 and m is 0. In an embodiment, n is 4 and m is 0. In an embodiment, n is 5 and m is 0. In an embodiment, n is 6 and m is 0. In an embodiment, n is 7 and m is 0. In an embodiment, n is 8 and m is 0. In an embodiment, n is 9 and m is 0. In an embodiment, n is 10 and m is 0.

In an embodiment, n is 0 and m is 1. In an embodiment, n is 1 and m is 1. In an embodiment, n is 2 and m is 1. In an embodiment, n is 3 and m is 1. In an embodiment, n is 4 and m is 1. In an embodiment, n is 5 and m is 1. In an embodiment, n is 6 and m is 1. In an embodiment, n is 7 and m is 1. In an embodiment, n is 8 and m is 1. In an embodiment, n is 9 and m is 1. In an embodiment, n is 10 and m is 1.

In an embodiment, n is 0 and m is 2. In an embodiment, n is 1 and m is 2. In an embodiment, n is 2 and m is 2. In an embodiment, n is 3 and m is 2. In an embodiment, n is 4 and m is 2. In an embodiment, n is 5 and m is 2. In an embodiment, n is 6 and m is 2. In an embodiment, n is 7 and m is 2. In an embodiment, n is 8 and m is 2. In an embodiment, n is 9 and m is 2. In an embodiment, n is 10 and m is 2.

In an embodiment, n is 0 and m is 3. In an embodiment, n is 1 and m is 3. In an embodiment, n is 2 and m is 3. In an embodiment, n is 3 and m is 3. In an embodiment, n is 4 and m is 3. In an embodiment, n is 5 and m is 3. In an embodiment, n is 6 and m is 3. In an embodiment, n is 7 and m is 3. In an embodiment, n is 8 and m is 3. In an embodiment, n is 9 and m is 3. In an embodiment, n is 10 and m is 3.

In an embodiment, n is 0 and m is 4. In an embodiment, n is 1 and m is 4. In an embodiment, n is 2 and m is 4. In an embodiment, n is 3 and m is 4. In an embodiment, n is 4 and m is 4. In an embodiment, n is 5 and m is 4. In an embodiment, n is 6 and m is 4. In an embodiment, n is 7 and m is 4. In an embodiment, n is 8 and m is 4. In an embodiment, n is 9 and m is 4. In an embodiment, n is 10 and m is 4.

In an embodiment, said agent bearing an N-Hydroxysuccinimide (NHS) moiety and an alkyne group is a compound of formula (IV):

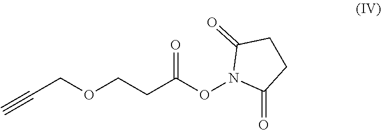 [SMILES: C#CCOCCC(=O)ON1C(=O)CCC1=O]

In an embodiment, step a) comprises reacting the saccharide with a carbonic acid derivative followed by reacting the carbonic acid derivative-activated saccharide with an azido linker in an aprotic solvent to produce an activated azido saccharide.

In one embodiment step a) comprises reacting the saccharide with an amount of carbonic acid derivative that is between 0.01-10 molar equivalent to the amount of saccharide present in the reaction mixture.

In one embodiment step a) comprises reacting the saccharide with an amount of carbonic acid derivative that is between 0.05-10 molar equivalent to the amount of saccharide present in the reaction mixture.

In one embodiment step a) comprises reacting the saccharide with an amount of carbonic acid derivative that is between 0.1-10 molar equivalent to the amount of saccharide present in the reaction mixture.

In one embodiment step a) comprises reacting the saccharide with an amount of carbonic acid derivative that is between 0.2-10 molar equivalent to the amount of saccharide present in the reaction mixture.

In one embodiment step a) comprises reacting the saccharide with an amount of carbonic acid derivative that is between 0.3-10 molar equivalent to the amount of saccharide present in the reaction mixture.

In one embodiment step a) comprises reacting the saccharide with an amount of carbonic acid derivative that is between 0.4-10 molar equivalent to the amount of saccharide present in the reaction mixture.

In one embodiment step a) comprises reacting the saccharide with an amount of carbonic acid derivative that is between 0.5-10 molar equivalent to the amount of saccharide present in the reaction mixture.

In one embodiment step a) comprises reacting the saccharide with an amount of carbonic acid derivative that is between 0.8-10 molar equivalent to the amount of saccharide present in the reaction mixture.

In one embodiment step a) comprises reacting the saccharide with an amount of carbonic acid derivative that is between 1-10 molar equivalent to the amount of saccharide present in the reaction mixture.

In one embodiment step a) comprises reacting the saccharide with an amount of carbonic acid derivative that is between 2-10 molar equivalent to the amount of saccharide present in the reaction mixture.

In one embodiment step a) comprises reacting the saccharide with an amount of carbonic acid derivative that is between 3-10 molar equivalent to the amount of saccharide present in the reaction mixture.

In one embodiment step a) comprises reacting the saccharide with an amount of carbonic acid derivative that is between 5-10 molar equivalent to the amount of saccharide present in the reaction mixture.

In one embodiment step a) comprises reacting the saccharide with an amount of carbonic acid derivative that is between 0.01-5 molar equivalent to the amount of saccharide present in the reaction mixture.

In one embodiment step a) comprises reacting the saccharide with an amount of carbonic acid derivative that is between 0.05-5 molar equivalent to the amount of saccharide present in the reaction mixture.

In one embodiment step a) comprises reacting the saccharide with an amount of carbonic acid derivative that is between 0.1-5 molar equivalent to the amount of saccharide present in the reaction mixture.

In one embodiment step a) comprises reacting the saccharide with an amount of carbonic acid derivative that is between 0.2-5 molar equivalent to the amount of saccharide present in the reaction mixture.

In one embodiment step a) comprises reacting the saccharide with an amount of carbonic acid derivative that is between 0.3-5 molar equivalent to the amount of saccharide present in the reaction mixture.

In one embodiment step a) comprises reacting the saccharide with an amount of carbonic acid derivative that is between 0.4-5 molar equivalent to the amount of saccharide present in the reaction mixture.

In one embodiment step a) comprises reacting the saccharide with an amount of carbonic acid derivative that is between 0.5-5 molar equivalent to the amount of saccharide present in the reaction mixture.

In one embodiment step a) comprises reacting the saccharide with an amount of carbonic acid derivative that is between 0.8-5 molar equivalent to the amount of saccharide present in the reaction mixture.

In one embodiment step a) comprises reacting the saccharide with an amount of carbonic acid derivative that is between 1-5 molar equivalent to the amount of saccharide present in the reaction mixture.

In one embodiment step a) comprises reacting the saccharide with an amount of carbonic acid derivative that is between 2-5 molar equivalent to the amount of saccharide present in the reaction mixture.

In one embodiment step a) comprises reacting the saccharide with an amount of carbonic acid derivative that is between 3-5 molar equivalent to the amount of saccharide present in the reaction mixture.

In one embodiment step a) comprises reacting the saccharide with an amount of carbonic acid derivative that is between 0.01-3 molar equivalent to the amount of saccharide present in the reaction mixture.

In one embodiment step a) comprises reacting the saccharide with an amount of carbonic acid derivative that is between 0.05-3 molar equivalent to the amount of saccharide present in the reaction mixture.

In one embodiment step a) comprises reacting the saccharide with an amount of carbonic acid derivative that is between 0.1-3 molar equivalent to the amount of saccharide present in the reaction mixture.

In one embodiment step a) comprises reacting the saccharide with an amount of carbonic acid derivative that is between 0.2-3 molar equivalent to the amount of saccharide present in the reaction mixture.

In one embodiment step a) comprises reacting the saccharide with an amount of carbonic acid derivative that is between 0.3-3 molar equivalent to the amount of saccharide present in the reaction mixture.

In one embodiment step a) comprises reacting the saccharide with an amount of carbonic acid derivative that is between 0.4-3 molar equivalent to the amount of saccharide present in the reaction mixture.

In one embodiment step a) comprises reacting the saccharide with an amount of carbonic acid derivative that is between 0.5-3 molar equivalent to the amount of saccharide present in the reaction mixture.

In one embodiment step a) comprises reacting the saccharide with an amount of carbonic acid derivative that is between 0.8-3 molar equivalent to the amount of saccharide present in the reaction mixture.

In one embodiment step a) comprises reacting the saccharide with an amount of carbonic acid derivative that is between 1-3 molar equivalent to the amount of saccharide present in the reaction mixture.

In one embodiment step a) comprises reacting the saccharide with an amount of carbonic acid derivative that is between 2-3 molar equivalent to the amount of saccharide present in the reaction mixture.

In one embodiment step a) comprises reacting the saccharide with an amount of carbonic acid derivative that is between 0.01-2 molar equivalent to the amount of saccharide present in the reaction mixture.

In one embodiment step a) comprises reacting the saccharide with an amount of carbonic acid derivative that is between 0.05-2 molar equivalent to the amount of saccharide present in the reaction mixture.

In one embodiment step a) comprises reacting the saccharide with an amount of carbonic acid derivative that is between 0.1-2 molar equivalent to the amount of saccharide present in the reaction mixture.

In one embodiment step a) comprises reacting the saccharide with an amount of carbonic acid derivative that is between 0.2-2 molar equivalent to the amount of saccharide present in the reaction mixture.

In one embodiment step a) comprises reacting the saccharide with an amount of carbonic acid derivative that is between 0.3-2 molar equivalent to the amount of saccharide present in the reaction mixture.

In one embodiment step a) comprises reacting the saccharide with an amount of carbonic acid derivative that is between 0.4-2 molar equivalent to the amount of saccharide present in the reaction mixture.

In one embodiment step a) comprises reacting the saccharide with an amount of carbonic acid derivative that is between 0.5-2 molar equivalent to the amount of saccharide present in the reaction mixture.

In one embodiment step a) comprises reacting the saccharide with an amount of carbonic acid derivative that is between 0.8-2 molar equivalent to the amount of saccharide present in the reaction mixture.

In one embodiment step a) comprises reacting the saccharide with an amount of carbonic acid derivative that is between 1-2 molar equivalent to the amount of saccharide present in the reaction mixture.

In one embodiment step a) comprises reacting the saccharide with an amount of carbonic acid derivative that is between 0.01-1 molar equivalent to the amount of saccharide present in the reaction mixture.

In one embodiment step a) comprises reacting the saccharide with an amount of carbonic acid derivative that is between 0.05-1 molar equivalent to the amount of saccharide present in the reaction mixture.

In one embodiment step a) comprises reacting the saccharide with an amount of carbonic acid derivative that is between 0.1-1 molar equivalent to the amount of saccharide present in the reaction mixture.

In one embodiment step a) comprises reacting the saccharide with an amount of carbonic acid derivative that is between 0.2-1 molar equivalent to the amount of saccharide present in the reaction mixture.

In one embodiment step a) comprises reacting the saccharide with an amount of carbonic acid derivative that is between 0.3-1 molar equivalent to the amount of saccharide present in the reaction mixture.

In one embodiment step a) comprises reacting the saccharide with an amount of carbonic acid derivative that is between 0.4-1 molar equivalent to the amount of saccharide present in the reaction mixture.

In one embodiment step a) comprises reacting the saccharide with an amount of carbonic acid derivative that is between 0.5-1 molar equivalent to the amount of saccharide present in the reaction mixture.

In one embodiment step a) comprises reacting the saccharide with an amount of carbonic acid derivative that is between 0.8-1 molar equivalent to the amount of saccharide present in the reaction mixture.

In one embodiment step a) comprises reacting the saccharide with an amount of carbonic acid derivative that is between 0.01-0.5 molar equivalent to the amount of saccharide present in the reaction mixture.

In one embodiment step a) comprises reacting the saccharide with an amount of carbonic acid derivative that is between 0.05-0.5 molar equivalent to the amount of saccharide present in the reaction mixture.

In one embodiment step a) comprises reacting the saccharide with an amount of carbonic acid derivative that is between 0.1-0.5 molar equivalent to the amount of saccharide present in the reaction mixture.

In one embodiment step a) comprises reacting the saccharide with an amount of carbonic acid derivative that is between 0.2-0.5 molar equivalent to the amount of saccharide present in the reaction mixture.

In one embodiment step a) comprises reacting the saccharide with an amount of carbonic acid derivative that is between 0.3-0.5 molar equivalent to the amount of saccharide present in the reaction mixture.

In one embodiment step a) comprises reacting the saccharide with an amount of carbonic acid derivative that is between 0.4-0.5 molar equivalent to the amount of saccharide present in the reaction mixture.

In one embodiment step a) comprises reacting the saccharide with an amount of carbonic acid derivative

In one embodiment step a) comprises reacting the saccharide with an amount of carbonic acid derivative that is between 0.01-0.4 molar equivalent to the amount of saccharide present in the reaction mixture.

In one embodiment step a) comprises reacting the saccharide with an amount of carbonic acid derivative that is between 0.05-0.4 molar equivalent to the amount of saccharide present in the reaction mixture.

In one embodiment step a) comprises reacting the saccharide with an amount of carbonic acid derivative that is between 0.1-0.4 molar equivalent to the amount of saccharide present in the reaction mixture.

In one embodiment step a) comprises reacting the saccharide with an amount of carbonic acid derivative that is between 0.2-0.4 molar equivalent to the amount of saccharide present in the reaction mixture.

In one embodiment step a) comprises reacting the saccharide with an amount of carbonic acid derivative that is between 0.3-0.4 molar equivalent to the amount of saccharide present in the reaction mixture.

In one embodiment step a) comprises reacting the saccharide with an amount of carbonic acid derivative that is between 0.01-0.3 molar equivalent to the amount of saccharide present in the reaction mixture.

In one embodiment step a) comprises reacting the saccharide with an amount of carbonic acid derivative that is between 0.05-0.3 molar equivalent to the amount of saccharide present in the reaction mixture.

In one embodiment step a) comprises reacting the saccharide with an amount of carbonic acid derivative that is between 0.1-0.3 molar equivalent to the amount of saccharide present in the reaction mixture.

In one embodiment step a) comprises reacting the saccharide with an amount of carbonic acid derivative that is between 0.2-0.3 molar equivalent to the amount of saccharide present in the reaction mixture.

In one embodiment step a) comprises reacting the saccharide with an amount of carbonic acid derivative of about 0.01 molar equivalent to the amount of saccharide present in the reaction mixture.

In one embodiment step a) comprises reacting the saccharide with an amount of carbonic acid derivative of about 0.05 molar equivalent to the amount of saccharide present in the reaction mixture.

In one embodiment step a) comprises reacting the saccharide with an amount of carbonic acid derivative of about 0.08 molar equivalent to the amount of saccharide present in the reaction mixture.

In one embodiment step a) comprises reacting the saccharide with an amount of carbonic acid derivative of about 0.1 molar equivalent to the amount of saccharide present in the reaction mixture.

In one embodiment step a) comprises reacting the saccharide with an amount of carbonic acid derivative of about 0.2 molar equivalent to the amount of saccharide present in the reaction mixture.

In one embodiment step a) comprises reacting the saccharide with an amount of carbonic acid derivative of about 0.3 molar equivalent to the amount of saccharide present in the reaction mixture.

In one embodiment step a) comprises reacting the saccharide with an amount of carbonic acid derivative of about 0.4 molar equivalent to the amount of saccharide present in the reaction mixture.

In one embodiment step a) comprises reacting the saccharide with an amount of carbonic acid derivative of about 0.5 molar equivalent to the amount of saccharide present in the reaction mixture.

In one embodiment step a) comprises reacting the saccharide with an amount of carbonic acid derivative of about 1 molar equivalent to the amount of saccharide present in the reaction mixture.

In one embodiment step a) comprises reacting the saccharide with an amount of carbonic acid derivative of about 2 molar equivalent to the amount of saccharide present in the reaction mixture.

In one embodiment step a) comprises reacting the saccharide with an amount of carbonic acid derivative of about 3 molar equivalent to the amount of saccharide present in the reaction mixture.

In one embodiment step a) comprises reacting the saccharide with an amount of carbonic acid derivative of about 4 molar equivalent to the amount of saccharide present in the reaction mixture.

In one embodiment step a) comprises reacting the saccharide with an amount of carbonic acid derivative of about 5 molar equivalent to the amount of saccharide present in the reaction mixture.

In one embodiment step a) comprises reacting the saccharide with an amount of carbonic acid derivative of about 8 molar equivalent to the amount of saccharide present in the reaction mixture.

In one embodiment step a) comprises reacting the saccharide with an amount of carbonic acid derivative of about 10 molar equivalent to the amount of saccharide present in the reaction mixture.

In an embodiment, at step a) the isolated saccharide is reacted with a carbonic acid derivative in an aprotic solvent.

In one embodiment the isolated saccharide is reacted with a carbonic acid derivative in a solution consisting essentially of dimethylsulphoxide (DMSO) or dimethylformamide (DMF). In one embodiment the isolated saccharide is reacted with a carbonic acid derivative in a solution consisting essentially of dimethylformamide (DMF).

In one embodiment the isolated saccharide is reacted with a carbonic acid derivative in a solution consisting essentially of dimethylsulphoxide (DMSO).

In an embodiment, the isolated saccharide is reacted with a carbonic acid derivative in a solution consisting essentially of dimethylacetamide. In an embodiment, the isolated saccharide is reacted with a carbonic acid derivative in a solution consisting essentially of N-methyl-2-pyrrolidone. In an embodiment, the isolated saccharide is reacted with a carbonic acid derivative in a solution consisting essentially of hexamethylphosphoramide (HMPA).

In a preferred embodiment the isolated saccharide is reacted with a carbonic acid derivative in a solution consisting essentially of dimethylsulphoxide (DMSO).

In one embodiment the isolated saccharide is reacted with a carbonic acid derivative in dimethylsulphoxide (DMSO) or dimethylformamide (DMF). In one embodiment the isolated saccharide is reacted with a carbonic acid derivative in dimethylformamide (DMF). In one embodiment the isolated saccharide is reacted with a carbonic acid derivative in dimethylsulphoxide (DMSO).

In an embodiment, the isolated saccharide is reacted with a carbonic acid derivative in dimethylacetamide. In an embodiment, the isolated saccharide is reacted with a carbonic acid derivative in N-methyl-2-pyrrolidone. In an embodiment, the isolated saccharide is reacted with a carbonic acid derivative in hexamethylphosphoramide (HMPA).

In a preferred embodiment the isolated saccharide is reacted with CDI in dimethylsulphoxide (DMSO). In an embodiment the isolated saccharide is reacted with CDI in anhydrous DMSO.

It has been surprisingly found that reacting the isolated saccharide with CDI in an environment with a moisture level of about 0.1% to 1% (v/v) allows to avoid side reactions.

Therefore, in one embodiment the isolated saccharide is reacted with CDI in an aprotic solvent comprising 0.1% to 1% (v/v) water. In one embodiment the isolated saccharide is reacted with CDI in an aprotic solvent comprising 0.1% to 0.8% (v/v) water. In one embodiment the isolated saccharide is reacted with CDI in an aprotic solvent comprising 0.1% to 0.5% (v/v) water. In one embodiment the isolated saccharide is reacted with CDI in an aprotic solvent comprising 0.1% to 0.4% (v/v) water. In one embodiment the isolated saccharide is reacted with CDI in an aprotic solvent comprising 0.1% to 0.3% (v/v) water.

In one embodiment the isolated saccharide is reacted with CDI in an aprotic solvent comprising 0.1% to 0.2% (v/v) water.

In one embodiment the isolated saccharide is reacted with CDI in an aprotic solvent comprising 0.2% to 1% (v/v) water. In one embodiment the isolated saccharide is reacted with CDI in an aprotic solvent comprising 0.2% to 0.8% (v/v) water. In one embodiment the isolated saccharide is reacted with CDI in an aprotic solvent comprising 0.2% to 0.5% (v/v) water. In one embodiment the isolated saccharide is reacted with CDI in an aprotic solvent comprising 0.2% to 0.4% (v/v) water. In one embodiment the isolated saccharide is reacted with CDI in an aprotic solvent comprising 0.2% to 0.3% (v/v) water.

In one embodiment the isolated saccharide is reacted with CDI in an aprotic solvent comprising 0.3% to 0.8% (v/v) water. In one embodiment the isolated saccharide is reacted with CDI in an aprotic solvent comprising 0.3% to 0.5% (v/v) water. In one embodiment the isolated saccharide is reacted with CDI in an aprotic solvent comprising 0.3% to 0.4% (v/v) water.

In one embodiment the isolated saccharide is reacted with CDI in an aprotic solvent comprising about 0.1% (v/v) water. In one embodiment the isolated saccharide is reacted with CDI in an aprotic solvent comprising about 0.2% (v/v) water. In one embodiment the isolated saccharide is reacted with CDI in an aprotic solvent comprising about 0.3% (v/v) water. In one embodiment the isolated saccharide is reacted with CDI in an aprotic solvent comprising about 0.4% (v/v) water. In one embodiment the isolated saccharide is reacted with CDI in an aprotic solvent comprising about 0.5% (v/v) water. In one embodiment the isolated saccharide is reacted with CDI in an aprotic solvent comprising about 0.6% (v/v) water. In one embodiment the isolated saccharide is reacted with CDI in an aprotic solvent comprising about 0.7% (v/v) water. In one embodiment the isolated saccharide is reacted with CDI in an aprotic solvent comprising about 0.8% (v/v) water. In one embodiment the isolated saccharide is reacted with CDI in an aprotic solvent comprising about 0.9% (v/v) water.

In one embodiment the isolated saccharide is reacted with CDI in DMSO comprising 0.1% to 1% (v/v) water. In one embodiment the isolated saccharide is reacted with CDI in DMSO comprising 0.1% to 0.8% (v/v) water. In one embodiment the isolated saccharide is reacted with CDI in DMSO comprising 0.1% to 0.5% (v/v) water. In one embodiment the isolated saccharide is reacted with CDI in DMSO comprising 0.1% to 0.4% (v/v) water. In one embodiment the isolated saccharide is reacted with CDI in DMSO comprising 0.1% to 0.3% (v/v) water. In one embodiment the isolated saccharide is reacted with CDI in DMSO comprising 0.1% to 0.2% (v/v) water.

In one embodiment the isolated saccharide is reacted with CDI in DMSO comprising 0.2% to 1% (v/v) water. In one embodiment the isolated saccharide is reacted with CDI in DMSO comprising 0.2% to 0.8% (v/v) water. In one embodiment the isolated saccharide is reacted with CDI in DMSO comprising 0.2% to 0.5% (v/v) water. In one embodiment the isolated saccharide is reacted with CDI in DMSO comprising 0.2% to 0.4% (v/v) water. In one embodiment the isolated saccharide is reacted with CDI in DMSO comprising 0.2% to 0.3% (v/v) water.

In one embodiment the isolated saccharide is reacted with CDI in DMSO comprising 0.3% to 0.8% (v/v) water. In one embodiment the isolated saccharide is reacted with CDI in DMSO comprising 0.3% to 0.5% (v/v) water. In one embodiment the isolated saccharide is reacted with CDI in DMSO comprising 0.3% to 0.4% (v/v) water.

In one embodiment the isolated saccharide is reacted with CDI in DMSO comprising about 0.1% (v/v) water. In one embodiment the isolated saccharide is reacted with CDI in DMSO comprising about 0.2% (v/v) water. In one embodiment the isolated saccharide is reacted with CDI in DMSO comprising about 0.3% (v/v) water. In one embodiment the isolated saccharide is reacted with CDI in DMSO comprising about 0.4% (v/v) water. In one embodiment the isolated saccharide is reacted with CDI in DMSO comprising about 0.5% (v/v) water. In one embodiment the isolated saccharide is reacted with CDI in DMSO comprising about 0.6% (v/v) water. In one embodiment the isolated saccharide is reacted with CDI in DMSO comprising about 0.7% (v/v) water. In one embodiment the isolated saccharide is reacted with CDI in DMSO comprising about 0.8% (v/v) water. In one embodiment the isolated saccharide is reacted with CDI in DMSO comprising about 0.9% (v/v) water.

In one embodiment the free carbonic acid derivative is then quenched by the addition of water before the addition of the azido linker. Water can inactivate free CDI.

Therefore, in an embodiment, carbonic acid derivative activation is followed by the addition of water. In an embodiment, water is added to bring the total water content in the mixture to between about 1% to about 10% (v/v). In an embodiment, water is added to bring the total water content in the mixture to between about 1.2% to about 8% (v/v). In an embodiment, water is added to bring the total water content in the mixture to between about 1.5% to about 5% (v/v). In an embodiment, water is added to bring the total water content in the mixture to between about 1.5% to about 3% (v/v). In an embodiment, water is added to bring the total water content in the mixture to between about 1.5% to about 2.5% (v/v). In an embodiment, water is added to bring the total water content in the mixture to about 1% (v/v). In an embodiment, water is added to bring the total water content in the mixture to about 1.2% (v/v). In an embodiment, water is added to bring the total water content in the mixture to about 1.4% (v/v). In an embodiment, water is added to bring the total water content in the mixture to about 1.5% (v/v). In an embodiment, water is added to bring the total water content in the mixture to about 2% (v/v). In an embodiment, water is added to bring the total water content in the mixture to about 2.5% (v/v). In an embodiment, water is added to bring the total water content in the mixture to about 3% (v/v). In an embodiment, water is added to bring the total water content in the mixture to about 5% (v/v). In an embodiment, water is added to bring the total water content in the mixture to about 7% (v/v). In an embodiment, water is added to bring the total water content in the mixture to about 10% (v/v).

Once the saccharide has been reacted with carbonic acid derivative and following an eventual quenching of carbonic acid derivative with water, the carbonic acid derivative-activated saccharide is reacted with an azido linker.

In one embodiment step a) further comprises reacting the carbonic acid derivative-activated saccharide with an amount of azido linker that is between 0.01-10 molar equivalent to the amount of polysaccharide Repeat Unit of the activated saccharide (molar equivalent of RU).

In one embodiment step a) further comprises reacting the carbonic acid derivative-activated saccharide with an amount of azido linker that is between 0.01-8 molar equivalent to the amount of polysaccharide repeat unit of the activated saccharide.

In one embodiment step a) further comprises reacting the carbonic acid derivative-activated saccharide with an amount of azido linker that is between 0.01-5 molar equivalent to the amount of polysaccharide repeat unit of the activated saccharide.

In one embodiment step a) further comprises reacting the carbonic acid derivative-activated saccharide with an amount of azido linker that is between 0.01-4 molar equivalent to the amount of polysaccharide repeat unit of the activated saccharide.

In one embodiment step a) further comprises reacting the carbonic acid derivative-activated saccharide with an amount of azido linker that is between 0.01-3 molar equivalent to the amount of polysaccharide repeat unit of the activated saccharide.

In one embodiment step a) further comprises reacting the carbonic acid derivative-activated saccharide with an amount of azido linker that is between 0.01-2 molar equivalent to the amount of polysaccharide repeat unit of the activated saccharide.

In one embodiment step a) further comprises reacting the carbonic acid derivative-activated saccharide with an amount of azido linker that is between 0.01-1 molar equivalent to the amount of polysaccharide repeat unit of the activated saccharide.

In one embodiment step a) further comprises reacting the carbonic acid derivative-activated saccharide with an amount of azido linker that is between 0.01-0.5 molar equivalent to the amount of polysaccharide repeat unit of the activated saccharide.

In one embodiment step a) further comprises reacting the carbonic acid derivative-activated saccharide with an amount of azido linker that is between 0.01-0.1 molar equivalent to the amount of polysaccharide repeat unit of the activated saccharide.

In one embodiment step a) further comprises reacting the carbonic acid derivative-activated saccharide with an amount of azido linker that is between 0.05-10 molar equivalent to the amount of polysaccharide repeat unit of the activated saccharide.

In one embodiment step a) further comprises reacting the carbonic acid derivative-activated saccharide with an amount of azido linker that is between 0.05-8 molar equivalent to the amount of polysaccharide repeat unit of the activated saccharide.

In one embodiment step a) further comprises reacting the carbonic acid derivative-activated saccharide with an amount of azido linker that is between 0.05-5 molar equivalent to the amount of polysaccharide repeat unit of the activated saccharide.

In one embodiment step a) further comprises reacting the carbonic acid derivative-activated saccharide with an amount of azido linker that is between 0.05-4 molar equivalent to the amount of polysaccharide repeat unit of the activated saccharide.

In one embodiment step a) further comprises reacting the carbonic acid derivative-activated saccharide with an amount of azido linker that is between 0.05-3 molar equivalent to the amount of polysaccharide repeat unit of the activated saccharide.

In one embodiment step a) further comprises reacting the carbonic acid derivative-activated saccharide with an amount of azido linker that is between 0.05-2 molar equivalent to the amount of polysaccharide repeat unit of the activated saccharide.

In one embodiment step a) further comprises reacting the carbonic acid derivative-activated saccharide with an amount of azido linker that is between 0.05-1 molar equivalent to the amount of polysaccharide repeat unit of the activated saccharide.

In one embodiment step a) further comprises reacting the carbonic acid derivative-activated saccharide with an amount of azido linker that is between 0.05-0.5 molar equivalent to the amount of polysaccharide repeat unit of the activated saccharide.

In one embodiment step a) further comprises reacting the carbonic acid derivative-activated saccharide with an amount of azido linker that is between 0.05-0.1 molar equivalent to the amount of polysaccharide repeat unit of the activated saccharide.

In one embodiment step a) further comprises reacting the carbonic acid derivative-activated saccharide with an amount of azido linker that is between 0.1-10 molar equivalent to the amount of polysaccharide repeat unit of the activated saccharide.

In one embodiment step a) further comprises reacting the carbonic acid derivative-activated saccharide with an amount of azido linker that is between 0.1-8 molar equivalent to the amount of polysaccharide repeat unit of the activated saccharide.

In one embodiment step a) further comprises reacting the carbonic acid derivative-activated saccharide with an amount of azido linker that is between 0.1-5 molar equivalent to the amount of polysaccharide repeat unit of the activated saccharide.

In one embodiment step a) further comprises reacting the carbonic acid derivative-activated saccharide with an amount of azido linker that is between 0.1-4 molar equivalent to the amount of polysaccharide repeat unit of the activated saccharide.

In one embodiment step a) further comprises reacting the carbonic acid derivative-activated saccharide with an amount of azido linker that is between 0.1-3 molar equivalent to the amount of polysaccharide repeat unit of the activated saccharide.

In one embodiment step a) further comprises reacting the carbonic acid derivative-activated saccharide with an amount of azido linker that is between 0.1-2 molar equivalent to the amount of polysaccharide repeat unit of the activated saccharide.

In one embodiment step a) further comprises reacting the carbonic acid derivative-activated saccharide with an amount of azido linker that is between 0.1-1 molar equivalent to the amount of polysaccharide repeat unit of the activated saccharide.

In one embodiment step a) further comprises reacting the carbonic acid derivative-activated saccharide with an amount of azido linker that is between 0.1-0.5 molar equivalent to the amount of polysaccharide repeat unit of the activated saccharide.

In one embodiment step a) further comprises reacting the carbonic acid derivative-activated saccharide with an amount of azido linker that is between 0.5-10 molar equivalent to the amount of polysaccharide repeat unit of the activated saccharide.

In one embodiment step a) further comprises reacting the carbonic acid derivative-activated saccharide with an amount of azido linker that is between 0.5-8 molar equivalent to the amount of polysaccharide repeat unit of the activated saccharide.

In one embodiment step a) further comprises reacting the carbonic acid derivative-activated saccharide with an amount of azido linker that is between 0.5-5 molar equivalent to the amount of polysaccharide repeat unit of the activated saccharide.

In one embodiment step a) further comprises reacting the carbonic acid derivative-activated saccharide with an amount of azido linker that is between 0.5-4 molar equivalent to the amount of polysaccharide repeat unit of the activated saccharide.

In one embodiment step a) further comprises reacting the carbonic acid derivative-activated saccharide with an amount of azido linker that is between 0.5-3 molar equivalent to the amount of polysaccharide repeat unit of the activated saccharide.

In one embodiment step a) further comprises reacting the carbonic acid derivative-activated saccharide with an amount of azido linker that is between 0.5-2 molar equivalent to the amount of polysaccharide repeat unit of the activated saccharide.

In one embodiment step a) further comprises reacting the carbonic acid derivative-activated saccharide with an amount of azido linker that is between 0.5-1 molar equivalent to the amount of polysaccharide repeat unit of the activated saccharide.

In one embodiment step a) further comprises reacting the carbonic acid derivative-activated saccharide with an amount of azido linker that is between 1-10 molar equivalent to the amount of polysaccharide repeat unit of the activated saccharide.

In one embodiment step a) further comprises reacting the carbonic acid derivative-activated saccharide with an amount of azido linker that is between 1-8 molar equivalent to the amount of polysaccharide repeat unit of the activated saccharide.

In one embodiment step a) further comprises reacting the carbonic acid derivative-activated saccharide with an amount of azido linker that is between 1-5 molar equivalent to the amount of polysaccharide repeat unit of the activated saccharide.

In one embodiment step a) further comprises reacting the carbonic acid derivative-activated saccharide with an amount of azido linker that is between 1-4 molar equivalent to the amount of polysaccharide repeat unit of the activated saccharide.

In one embodiment step a) further comprises reacting the carbonic acid derivative-activated saccharide with an amount of azido linker that is between 1-3 molar equivalent to the amount of polysaccharide repeat unit of the activated saccharide.

In one embodiment step a) further comprises reacting the carbonic acid derivative-activated saccharide with an amount of azido linker that is between 1-2 molar equivalent to the amount of polysaccharide repeat unit of the activated saccharide.

In one embodiment step a) further comprises reacting the carbonic acid derivative-activated saccharide with an amount of azido linker that is between 2-10 molar equivalent to the amount of polysaccharide repeat unit of the activated saccharide.

In one embodiment step a) further comprises reacting the carbonic acid derivative-activated saccharide with an amount of azido linker that is between 2-8 molar equivalent to the amount of polysaccharide repeat unit of the activated saccharide.

In one embodiment step a) further comprises reacting the carbonic acid derivative-activated saccharide with an amount of azido linker that is between 2-5 molar equivalent to the amount of polysaccharide repeat unit of the activated saccharide.

In one embodiment step a) further comprises reacting the carbonic acid derivative-activated saccharide with an amount of azido linker that is between 2-4 molar equivalent to the amount of polysaccharide repeat unit of the activated saccharide.

In one embodiment step a) further comprises reacting the carbonic acid derivative-activated saccharide with an amount of azido linker that is between 2-3 molar equivalent to the amount of polysaccharide repeat unit of the activated saccharide.

In one embodiment step a) further comprises reacting the carbonic acid derivative-activated saccharide with an amount of azido linker that is between 3-10 molar equivalent to the amount of polysaccharide repeat unit of the activated saccharide.

In one embodiment step a) further comprises reacting the carbonic acid derivative-activated saccharide with an amount of azido linker that is between 3-8 molar equivalent to the amount of polysaccharide repeat unit of the activated saccharide.

In one embodiment step a) further comprises reacting the carbonic acid derivative-activated saccharide with an amount of azido linker that is between 3-5 molar equivalent to the amount of polysaccharide repeat unit of the activated saccharide.

In one embodiment step a) further comprises reacting the carbonic acid derivative-activated saccharide with an amount of azido linker that is between 3-4 molar equivalent to the amount of polysaccharide repeat unit of the activated saccharide.

In one embodiment step a) further comprises reacting the carbonic acid derivative-activated saccharide with an amount of azido linker that is between 4-10 molar equivalent to the amount of polysaccharide repeat unit of the activated saccharide.

In one embodiment step a) further comprises reacting the carbonic acid derivative-activated saccharide with an amount of azido linker that is between 4-8 molar equivalent to the amount of polysaccharide repeat unit of the activated saccharide.

In one embodiment step a) further comprises reacting the carbonic acid derivative-activated saccharide with an amount of azido linker that is between 4-5 molar equivalent to the amount of polysaccharide repeat unit of the activated saccharide.

In one embodiment step a) further comprises reacting the carbonic acid derivative-activated saccharide with an amount of azido linker that is between 5-10 molar equivalent to the amount of polysaccharide repeat unit of the activated saccharide.

In one embodiment step a) further comprises reacting the carbonic acid derivative-activated saccharide with an amount of azido linker that is between 5-8 molar equivalent to the amount of polysaccharide repeat unit of the activated saccharide.

In one embodiment step a) further comprises reacting the carbonic acid derivative-activated saccharide with an amount of azido linker that is between 8-10 molar equivalent to the amount of polysaccharide repeat unit of the activated saccharide.

In one embodiment step a) further comprises reacting the carbonic acid derivative-activated saccharide with an amount of azido linker that is about 0.01 molar equivalent to the amount of polysaccharide repeat unit of the activated saccharide.

In one embodiment step a) further comprises reacting the carbonic acid derivative-activated saccharide with an amount of azido linker that is about 0.05 molar equivalent to the amount of polysaccharide repeat unit of the activated saccharide.

In one embodiment step a) further comprises reacting the carbonic acid derivative-activated saccharide with an amount of azido linker that is about 0.1 molar equivalent to the amount of polysaccharide repeat unit of the activated saccharide.

In one embodiment step a) further comprises reacting the carbonic acid derivative-activated saccharide with an amount of azido linker that is about 0.5 molar equivalent to the amount of polysaccharide repeat unit of the activated saccharide.

In one embodiment step a) further comprises reacting the carbonic acid derivative-activated saccharide with an amount of azido linker that is about 1 molar equivalent to the amount of polysaccharide repeat unit of the activated saccharide.

In one embodiment step a) further comprises reacting the carbonic acid derivative-activated saccharide with an amount of azido linker that is about 2 molar equivalents to the amount of polysaccharide repeat unit of the activated saccharide.

In one embodiment step a) further comprises reacting the carbonic acid derivative-activated saccharide with an amount of azido linker that is about 3 molar equivalents to the amount of polysaccharide repeat unit of the activated saccharide.

In one embodiment step a) further comprises reacting the carbonic acid derivative-activated saccharide with an amount of azido linker that is about 4 molar equivalents to the amount of polysaccharide repeat unit of the activated saccharide.

In one embodiment step a) further comprises reacting the carbonic acid derivative-activated saccharide with an amount of azido linker that is about 5 molar equivalents to the amount of polysaccharide repeat unit of the activated saccharide.

In one embodiment step a) further comprises reacting the carbonic acid derivative-activated saccharide with an amount of azido linker that is about 8 molar equivalents to the amount of polysaccharide repeat unit of the activated saccharide.

In one embodiment step a) further comprises reacting the carbonic acid derivative-activated saccharide with an amount of azido linker that is about 10 molar equivalents to the amount of polysaccharide repeat unit of the activated saccharide.

In the above embodiments, said carbonic acid derivative is preferably CDI. In another embodiment, said carbonic acid derivative is CDT.

In one embodiment the degree of activation of the activated saccharide following step a) is between 0.5 to 50%. The degree of activation of the azido saccharide being defined as the percentage of Repeating Unit linked to an azido linker.

In one embodiment the degree of activation of the activated saccharide following step a) is between 1 to 30%. In another embodiment the degree of activation of the activated saccharide following step a) is between 2 to 25%. In another embodiment the degree of activation of the activated saccharide following step a) is between 3 to 20%.

In another embodiment the degree of activation of the activated saccharide following step a) is between 3 to 15%. In another embodiment the degree of activation of the activated saccharide following step a) is between 4 to 15%. In an embodiment the degree of activation of the activated saccharide following step a) is between 1 to 6%.

In an embodiment the degree of activation of the activated saccharide following step a) is between 3 to 6%. In an embodiment the degree of activation of the activated saccharide following step a) is between 10 to 15%.

In an embodiment the degree of activation of the activated saccharide following step a) is about 1%. In an embodiment the degree of activation of the activated saccharide following step a) is about 2%. In an embodiment the degree of activation of the activated saccharide following step a) is about 3%. In an embodiment the degree of activation of the activated saccharide following step a) is about 4%. In an embodiment the degree of activation of the activated saccharide following step a) is about 5%. In an embodiment the degree of activation of the activated saccharide following step a) is about 6%. In an embodiment the degree of activation of the activated saccharide following step a) is about 7%. In an embodiment the degree of activation of the activated saccharide following step a) is about 8%. In an embodiment the degree of activation of the activated saccharide following step a) is about 9%. In an embodiment the degree of activation of the activated saccharide following step a) is about 10%. In an embodiment the degree of activation of the activated saccharide following step a) is about 11%. In an embodiment the degree of activation of the activated saccharide following step a) is about 12%. In an embodiment the degree of activation of the activated saccharide following step a) is about 13%. In an embodiment the degree of activation of the activated saccharide following step a) is about 14%. In an embodiment the degree of activation of the activated saccharide following step a) is about 15%. In an embodiment the degree of activation of the activated saccharide following step a) is about 16%. In an embodiment the degree of activation of the activated saccharide following step a) is about 17%. In an embodiment the degree of activation of the activated saccharide following step a) is about 18%. In an embodiment the degree of activation of the activated saccharide following step a) is about 19%. In an embodiment the degree of activation of the activated saccharide following step a) is about 20%.

In one embodiment step b) comprises reacting the carrier protein with an amount of agent bearing an N-Hydroxysuccinimide (NHS) moiety and an alkyne group that is 0.1-molar equivalents to the lysines on the carrier.

In one embodiment step b) comprises reacting the carrier protein with an amount of agent bearing an N-Hydroxysuccinimide (NHS) moiety and an alkyne group that is 0.5-molar equivalents to the lysines on the carrier.

In one embodiment step b) comprises reacting the carrier protein with an amount of agent bearing an N-Hydroxysuccinimide (NHS) moiety and an alkyne group that is 1-molar equivalents to the lysines on the carrier.

In one embodiment step b) comprises reacting the carrier protein with an amount of agent bearing an N-Hydroxysuccinimide (NHS) moiety and an alkyne group that is 1.5-molar equivalents to the lysines on the carrier.

In one embodiment step b) comprises reacting the carrier protein with an amount of agent bearing an N-Hydroxysuccinimide (NHS) moiety and an alkyne group that is 2-molar equivalents to the lysines on the carrier.

In one embodiment step b) comprises reacting the carrier protein with an amount of agent bearing an N-Hydroxysuccinimide (NHS) moiety and an alkyne group that is 2.5-molar equivalents to the lysines on the carrier.

In one embodiment step b) comprises reacting the carrier protein with an amount of agent bearing an N-Hydroxysuccinimide (NHS) moiety and an alkyne group that is 3-molar equivalents to the lysines on the carrier.

In one embodiment step b) comprises reacting the carrier protein with an amount of agent bearing an N-Hydroxysuccinimide (NHS) moiety and an alkyne group that is 5-molar equivalents to the lysines on the carrier.

In one embodiment step b) comprises reacting the carrier protein with an amount of agent bearing an N-Hydroxysuccinimide (NHS) moiety and an alkyne group that is 7.5-molar equivalents to the lysines on the carrier.

In one embodiment step b) comprises reacting the carrier protein with an amount of agent bearing an N-Hydroxysuccinimide (NHS) moiety and an alkyne group that is 0.1-7.5 molar equivalents to the lysines on the carrier.

In one embodiment step b) comprises reacting the carrier protein with an amount of agent bearing an N-Hydroxysuccinimide (NHS) moiety and an alkyne group that is 0.5-7.5 molar equivalents to the lysines on the carrier.

In one embodiment step b) comprises reacting the carrier protein with an amount of agent bearing an N-Hydroxysuccinimide (NHS) moiety and an alkyne group that is 1-7.5 molar equivalents to the lysines on the carrier.

In one embodiment step b) comprises reacting the carrier protein with an amount of agent bearing an N-Hydroxysuccinimide (NHS) moiety and an alkyne group that is 1.5-7.5 molar equivalents to the lysines on the carrier.

In one embodiment step b) comprises reacting the carrier protein with an amount of agent bearing an N-Hydroxysuccinimide (NHS) moiety and an alkyne group that is 2-7.5 molar equivalents to the lysines on the carrier.

In one embodiment step b) comprises reacting the carrier protein with an amount of agent bearing an N-Hydroxysuccinimide (NHS) moiety and an alkyne group that is 2.5-7.5 molar equivalents to the lysines on the carrier.

In one embodiment step b) comprises reacting the carrier protein with an amount of agent bearing an N-Hydroxysuccinimide (NHS) moiety and an alkyne group that is 3-7.5 molar equivalents to the lysines on the carrier.

In one embodiment step b) comprises reacting the carrier protein with an amount of agent bearing an N-Hydroxysuccinimide (NHS) moiety and an alkyne group that is 5-7.5 molar equivalents to the lysines on the carrier.

In one embodiment step b) comprises reacting the carrier protein with an amount of agent bearing an N-Hydroxysuccinimide (NHS) moiety and an alkyne group that is 0.1-molar equivalents to the lysines on the carrier.

In one embodiment step b) comprises reacting the carrier protein with an amount of agent bearing an N-Hydroxysuccinimide (NHS) moiety and an alkyne group that is 0.5-molar equivalents to the lysines on the carrier.

In one embodiment step b) comprises reacting the carrier protein with an amount of agent bearing an N-Hydroxysuccinimide (NHS) moiety and an alkyne group that is 1-5 molar equivalents to the lysines on the carrier.

In one embodiment step b) comprises reacting the carrier protein with an amount of agent bearing an N-Hydroxysuccinimide (NHS) moiety and an alkyne group that is 1.5-molar equivalents to the lysines on the carrier.

In one embodiment step b) comprises reacting the carrier protein with an amount of agent bearing an N-Hydroxysuccinimide (NHS) moiety and an alkyne group that is 2-5 molar equivalents to the lysines on the carrier.

In one embodiment step b) comprises reacting the carrier protein with an amount of agent bearing an N-Hydroxysuccinimide (NHS) moiety and an alkyne group that is 2.5-molar equivalents to the lysines on the carrier.

In one embodiment step b) comprises reacting the carrier protein with an amount of agent bearing an N-Hydroxysuccinimide (NHS) moiety and an alkyne group that is 3-5 molar equivalents to the lysines on the carrier.

In one embodiment step b) comprises reacting the carrier protein with an amount of agent bearing an N-Hydroxysuccinimide (NHS) moiety and an alkyne group that is 0.1-3 molar equivalents to the lysines on the carrier.

In one embodiment step b) comprises reacting the carrier protein with an amount of agent bearing an N-Hydroxysuccinimide (NHS) moiety and an alkyne group that is 0.5-3 molar equivalents to the lysines on the carrier.

In one embodiment step b) comprises reacting the carrier protein with an amount of agent bearing an N-Hydroxysuccinimide (NHS) moiety and an alkyne group that is 1-3 molar equivalents to the lysines on the carrier.

In one embodiment step b) comprises reacting the carrier protein with an amount of agent bearing an N-Hydroxysuccinimide (NHS) moiety and an alkyne group that is 1.5-3 molar equivalents to the lysines on the carrier.

In one embodiment step b) comprises reacting the carrier protein with an amount of agent bearing an N-Hydroxysuccinimide (NHS) moiety and an alkyne group that is 2-3 molar equivalents to the lysines on the carrier.

In one embodiment step b) comprises reacting the carrier protein with an amount of agent bearing an N-Hydroxysuccinimide (NHS) moiety and an alkyne group that is 2.5-3 molar equivalents to the lysines on the carrier.

In one embodiment step b) comprises reacting the carrier protein with an amount of agent bearing an N-Hydroxysuccinimide (NHS) moiety and an alkyne group that is 0.1-2.5 molar equivalents to the lysines on the carrier.

In one embodiment step b) comprises reacting the carrier protein with an amount of agent bearing an N-Hydroxysuccinimide (NHS) moiety and an alkyne group that is 0.5-2.5 molar equivalents to the lysines on the carrier.

In one embodiment step b) comprises reacting the carrier protein with an amount of agent bearing an N-Hydroxysuccinimide (NHS) moiety and an alkyne group that is 1-2.5 molar equivalents to the lysines on the carrier.

In one embodiment step b) comprises reacting the carrier protein with an amount of agent bearing an N-Hydroxysuccinimide (NHS) moiety and an alkyne group that is 1.5-2.5 molar equivalents to the lysines on the carrier.

In one embodiment step b) comprises reacting the carrier protein with an amount of agent bearing an N-Hydroxysuccinimide (NHS) moiety and an alkyne group that is 2-2.5 molar equivalents to the lysines on the carrier.

In one embodiment step b) comprises reacting the carrier protein with an amount of agent bearing an N-Hydroxysuccinimide (NHS) moiety and an alkyne group that is 0.1-2 molar equivalents to the lysines on the carrier.

In one embodiment step b) comprises reacting the carrier protein with an amount of agent bearing an N-Hydroxysuccinimide (NHS) moiety and an alkyne group that is 0.5-2 molar equivalents to the lysines on the carrier.

In one embodiment step b) comprises reacting the carrier protein with an amount of agent bearing an N-Hydroxysuccinimide (NHS) moiety and an alkyne group that is 1-2 molar equivalents to the lysines on the carrier.

In one embodiment step b) comprises reacting the carrier protein with an amount of agent bearing an N-Hydroxysuccinimide (NHS) moiety and an alkyne group that is 1.5-2 molar equivalents to the lysines on the carrier.

In one embodiment step b) comprises reacting the carrier protein with an amount of agent bearing an N-Hydroxysuccinimide (NHS) moiety and an alkyne group that is 0.1-1.5 molar equivalents to the lysines on the carrier.

In one embodiment step b) comprises reacting the carrier protein with an amount of agent bearing an N-Hydroxysuccinimide (NHS) moiety and an alkyne group that is 0.5-1.5 molar equivalents to the lysines on the carrier.

In one embodiment step b) comprises reacting the carrier protein with an amount of agent bearing an N-Hydroxysuccinimide (NHS) moiety and an alkyne group that is 1-1.5 molar equivalents to the lysines on the carrier.

In one embodiment step b) comprises reacting the carrier protein with an amount of agent bearing an N-Hydroxysuccinimide (NHS) moiety and an alkyne group that is 0.1-1 molar equivalents to the lysines on the carrier.

In one embodiment step b) comprises reacting the carrier protein with an amount of agent bearing an N-Hydroxysuccinimide (NHS) moiety and an alkyne group that is 0.5-1 molar equivalents to the lysines on the carrier.

In one embodiment step b) comprises reacting the carrier protein with an amount of agent bearing an N-Hydroxysuccinimide (NHS) moiety and an alkyne group that is 0.1-0.5 molar equivalents to the lysines on the carrier.

In one embodiment step b) comprises reacting the carrier protein with an amount of agent bearing an N-Hydroxysuccinimide (NHS) moiety and an alkyne group that is about 10 molar equivalents to the lysines on the carrier.

In one embodiment step b) comprises reacting the carrier protein with an amount of agent bearing an N-Hydroxysuccinimide (NHS) moiety and an alkyne group that is about 7.5 molar equivalents to the lysines on the carrier.

In one embodiment step b) comprises reacting the carrier protein with an amount of agent bearing an N-Hydroxysuccinimide (NHS) moiety and an alkyne group that is about 5 molar equivalents to the lysines on the carrier.

In one embodiment step b) comprises reacting the carrier protein with an amount of agent bearing an N-Hydroxysuccinimide (NHS) moiety and an alkyne group that is about 3 molar equivalents to the lysines on the carrier.

In one embodiment step b) comprises reacting the carrier protein with an amount of agent bearing an N-Hydroxysuccinimide (NHS) moiety and an alkyne group that is about 2.5 molar equivalents to the lysines on the carrier.

In one embodiment step b) comprises reacting the carrier protein with an amount of agent bearing an N-Hydroxysuccinimide (NHS) moiety and an alkyne group that is about 2 molar equivalents to the lysines on the carrier.

In one embodiment step b) comprises reacting the carrier protein with an amount of agent bearing an N-Hydroxysuccinimide (NHS) moiety and an alkyne group that is about 1.5 molar equivalents to the lysines on the carrier.

In one embodiment step b) comprises reacting the carrier protein with an amount of agent bearing an N-Hydroxysuccinimide (NHS) moiety and an alkyne group that is about 1 molar equivalent to the lysines on the carrier.

In one embodiment step b) comprises reacting the carrier protein with an amount of agent bearing an N-Hydroxysuccinimide (NHS) moiety and an alkyne group that is about 0.5 molar equivalents to the lysines on the carrier.

In one embodiment step b) comprises reacting the carrier protein with an amount of agent bearing an N-Hydroxysuccinimide (NHS) moiety and an alkyne group that is about 0.1 molar equivalents to the lysines on the carrier.

In one embodiment the degree of activation of the activated carrier following step b) is between 1 and 50. The degree of activation of the activated carrier being defined as the number of lysine residues in the carrier protein that become linked to the agent bearing an N-Hydroxysuccinimide (NHS) moiety and an alkyne group.

In an embodiment, the carrier protein is CRM₁₉₇, which contains 39 lysine residues. In said embodiment the degree of activation of the activated carrier following step b) may be between 1 to 30. In another embodiment the degree of activation of the activated carrier (CRM₁₉₇) following step b) is between 5 to 20. In another embodiment the degree of activation of the activated carrier (CRM₁₉₇) following step b) is between 9 to 18. In another embodiment the degree of activation of the activated carrier (CRM₁₉₇) following step b) is between 8 to 11. In another embodiment the degree of activation of the activated carrier (CRM₁₉₇) following step b) is between 15 to 20. In another embodiment the degree of activation of the activated carrier (CRM₁₉₇) following step b) is about 5. In another embodiment the degree of activation of the activated carrier (CRM₁₉₇) following step b) is about 6. In another embodiment the degree of activation of the activated carrier (CRM₁₉₇) following step b) is about 7. In another embodiment the degree of activation of the activated carrier (CRM₁₉₇) following step b) is about 8. In another embodiment the degree of activation of the activated carrier (CRM₁₉₇) following step b) is about 9. In another embodiment the degree of activation of the activated carrier (CRM₁₉₇) following step b) is about 10. In another embodiment the degree of activation of the activated carrier (CRM₁₉₇) following step b) is about 11. In another embodiment the degree of activation of the activated carrier (CRM₁₉₇) following step b) is about 12. In another embodiment the degree of activation of the activated carrier (CRM₁₉₇) following step b) is about 13. In another embodiment the degree of activation of the activated carrier (CRM₁₉₇) following step b) is about 14. In another embodiment the degree of activation of the activated carrier (CRM₁₉₇) following step b) is about 15. In another embodiment the degree of activation of the activated carrier (CRM₁₉₇) following step b) is about 16. In another embodiment the degree of activation of the activated carrier (CRM₁₉₇) following step b) is about 17. In another embodiment the degree of activation of the activated carrier (CRM₁₉₇) following step b) is about 18. In another embodiment the degree of activation of the activated carrier (CRM₁₉₇) following step b) is about 19. In another embodiment the degree of activation of the activated carrier (CRM₁₉₇) following step b) is about 20. In another embodiment the degree of activation of the activated carrier (CRM₁₉₇) following step b) is about 21. In another embodiment the degree of activation of the activated carrier (CRM₁₉₇) following step b) is about 22. In another embodiment the degree of activation of the activated carrier (CRM₁₉₇) following step b) is about 23. In another embodiment the degree of activation of the activated carrier (CRM₁₉₇) following step b) is about 24. In another embodiment the degree of activation of the activated carrier (CRM₁₉₇) following step b) is about 25.

In an embodiment, the carrier protein is SCP or a fragment thereof. In said embodiment the degree of activation of the activated carrier following step b) may be between 1 to 50. In another embodiment the degree of activation of the activated carrier (SCP) following step b) is between 5 to 50. In another embodiment the degree of activation of the activated carrier (SCP) following step b) is between 7 to 45. In another embodiment the degree of activation of the activated carrier (SCP) following step b) is between 5 to 15. In another embodiment the degree of activation of the activated carrier (SCP) following step b) is between 20 to 30. In another embodiment the degree of activation of the activated carrier (SCP) following step b) is between 30 to 50. In another embodiment the degree of activation of the activated carrier (SCP) following step b) is between 30 to 40. In another embodiment the degree of activation of the activated carrier (SCP) following step b) is between 10 to 40. In another embodiment the degree of activation of the activated carrier (SCP) following step b) is about 5. In another embodiment the degree of activation of the activated carrier (SCP) following step b) is about 7. In another embodiment the degree of activation of the activated carrier (SCP) following step b) is about 10. In another embodiment the degree of activation of the activated carrier (SCP) following step b) is about 13. In another embodiment the degree of activation of the activated carrier (SCP) following step b) is about 15. In another embodiment the degree of activation of the activated carrier (SCP) following step b) is about 20. In another embodiment the degree of activation of the activated carrier (SCP) following step b) is about 26. In another embodiment the degree of activation of the activated carrier (SCP) following step b) is about 30. In another embodiment the degree of activation of the activated carrier (SCP) following step b) is about 35. In another embodiment the degree of activation of the activated carrier (SCP) following step b) is about 37. In another embodiment the degree of activation of the activated carrier (SCP) following step b) is about 40. In another embodiment the degree of activation of the activated carrier (SCP) following step b) is about 45. In another embodiment the degree of activation of the activated carrier (SCP) following step b) is about 50.

In an embodiment, the carrier protein is TT or a fragment thereof. In said embodiment the degree of activation of the activated carrier following step b) may be between 1 to 30. In another embodiment the degree of activation of the activated carrier (TT) following step b) is between 5 to 25. In another embodiment the degree of activation of the activated carrier (TT) following step b) is between 7 to 25. In another embodiment the degree of activation of the activated carrier (TT) following step b) is between 10 to 20.

In another embodiment the degree of activation of the activated carrier (TT) following step b) is about 5. In another embodiment the degree of activation of the activated carrier (TT) following step b) is about 7. In another embodiment the degree of activation of the activated carrier (TT) following step b) is about 10. In another embodiment the degree of activation of the activated carrier (TT) following step b) is about 12. In another embodiment the degree of activation of the activated carrier (TT) following step b) is about 15. In another embodiment the degree of activation of the activated carrier (TT) following step b) is about 20. In another embodiment the degree of activation of the activated carrier (TT) following step b) is about 25. In another embodiment the degree of activation of the activated carrier (TT) following step b) is about 30.

In an embodiment, the conjugation reaction c) is carried out in aqueous buffer. In an embodiment, the conjugation reaction c) is carried out in aqueous buffer in the presence of copper (I) as catalyst. In an embodiment, the conjugation reaction c) is carried out in aqueous buffer in the presence an oxidant and of copper (I) as catalyst. In a preferred embodiment, the conjugation reaction c) is carried out in aqueous buffer in the presence of copper (1) as catalyst and ascorbate as oxidant. In an embodiment, THPTA (tris(3-hydroxypropyltriazolylmethyl)amine) and aminoguanidine may be further added to protect the protein from side reactions. Therefore, in a preferred embodiment, the conjugation reaction c) is carried out in aqueous buffer in the presence of copper (1) as catalyst and ascorbate as oxidant, wherein the reaction mixture further comprises THPTA (tris(3-hydroxypropyltriazolylmethyl)amine) and aminoguanidine.

In an embodiment the initial input ratio (weight by weight) of activated azido saccharide to activated alkyne-carrier at setp c) is between 0.1 and 3. In an embodiment the initial input ratio (weight by weight) of activated azido saccharide to activated alkyne-carrier at setp c) is between 0.5 and 2. In an embodiment the initial input ratio (weight by weight) of activated azido saccharide to activated alkyne-carrier at setp c) is between 0.6 and 1.5. In a preferred embodiment the initial input ratio (weight by weight) of activated azido saccharide to activated alkyne-carrier at setp c) is between 0.8 and 1. In an embodiment the initial input ratio (weight by weight) of activated azido saccharide to activated alkyne-carrier at setp c) is about 0.5. In an embodiment the initial input ratio (weight by weight) of activated azido saccharide to activated alkyne-carrier at setp c) is about 0.6. In an embodiment the initial input ratio (weight by weight) of activated azido saccharide to activated alkyne-carrier at setp c) is about 0.7. In an embodiment the initial input ratio (weight by weight) of activated azido saccharide to activated alkyne-carrier at setp c) is about 0.8. In an embodiment the initial input ratio (weight by weight) of activated azido saccharide to activated alkyne-carrier at setp c) is about 0.9. In an embodiment the initial input ratio (weight by weight) of activated azido saccharide to activated alkyne-carrier at setp c) is about 1. In an embodiment the initial input ratio (weight by weight) of activated azido saccharide to activated alkyne-carrier at setp c) is about 1.1. In an embodiment the initial input ratio (weight by weight) of activated azido saccharide to activated alkyne-carrier at setp c) is about 1.2. In an embodiment the initial input ratio (weight by weight) of activated azido saccharide to activated alkyne-carrier at setp c) is about 1.3. In an embodiment the initial input ratio (weight by weight) of activated azido saccharide to activated alkyne-carrier at setp c) is about 1.4. In an embodiment the initial input ratio (weight by weight) of activated azido saccharide to activated alkyne-carrier at setp c) is about 1.5. In an embodiment the initial input ratio (weight by weight) of activated azido saccharide to activated alkyne-carrier at setp c) is about 1.6. In an embodiment the initial input ratio (weight by weight) of activated azido saccharide to activated alkyne-carrier at setp c) is about 1.7. In an embodiment the initial input ratio (weight by weight) of activated azido saccharide to activated alkyne-carrier at setp c) is about 1.8. In an embodiment the initial input ratio (weight by weight) of activated azido saccharide to activated alkyne-carrier at setp c) is about 1.9. In an embodiment the initial input ratio (weight by weight) of activated azido saccharide to activated alkyne-carrier at setp c) is about 2.

Following the click conjugation reaction, there may remain unreacted azido groups in the conjugates, these may be capped using a suitable azido group capping agent. Therefore, in an embodiment, following step c), unreacted azido groups in the conjugates, are capped using a suitable azido group capping agent. In one embodiment this azido group capping agent is an agent bearing an alkyne group. In one embodiment this azido group capping agent is an agent bearing a terminal alkyne. In one embodiment this azido group capping agent is an agent bearing a cycloalkyne.

In an embodiment, said azido group capping agent is a compound of formula (V),

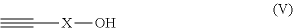

wherein X is (CH₂)ₙ wherein n is selected from 1 to 15.

In one embodiment this azido group capping agent is propargyl alcohol.

Therefore, in an embodiment, following step (c) the process further comprises a step of capping the unreacted azido groups remained in the conjugates with an azido group capping agent.

In an embodiment the capping of the unreacted azido groups is performed with an amount of capping agent that is between 0.05 to 20 molar equivalents to the amount of polysaccharide repeat unit of the activated saccharide.

In an embodiment the capping of the unreacted azido groups is performed with an amount of capping agent that is between 0.1 to 15 molar equivalents to the amount of polysaccharide repeat unit of the activated saccharide.

In an embodiment the capping of the unreacted azido groups is performed with an amount of capping agent that is between 0.5 to 10 molar equivalents to the amount of polysaccharide repeat unit of the activated saccharide.

In an embodiment the capping of the unreacted azido groups is performed with an amount of capping agent that is between 0.5 to 5 molar equivalents to the amount of polysaccharide repeat unit of the activated saccharide.

In an embodiment the capping of the unreacted azido groups is performed with an amount of capping agent that is between 0.5 to 2 molar equivalents to the amount of polysaccharide repeat unit of the activated saccharide.

In an embodiment the capping of the unreacted azido groups is performed with an amount of capping agent that is between 0.5 to 1 molar equivalents to the amount of polysaccharide repeat unit of the activated saccharide.

In an embodiment the capping of the unreacted azido groups is performed with an amount of capping agent that is between 1 to 2 molar equivalents to the amount of polysaccharide repeat unit of the activated saccharide.

In an embodiment the capping of the unreacted azido groups is performed with an amount of capping agent that is between 0.75 to 1.5 molar equivalents to the amount of polysaccharide repeat unit of the activated saccharide.

In an embodiment the capping of the unreacted azido groups is performed with an amount of capping agent that is about 1 molar equivalent to the amount of polysaccharide repeat unit of the activated saccharide.

In an embodiment the capping of the unreacted azido groups is performed with an amount of capping agent that is about 1.5 molar equivalent to the amount of polysaccharide repeat unit of the activated saccharide.

In an embodiment the capping of the unreacted azido groups is performed with an amount of capping agent that is about 0.5 molar equivalent to the amount of polysaccharide repeat unit of the activated saccharide.

In an embodiment the capping of the unreacted azido groups is performed with an amount of capping agent that is about 2 molar equivalents to the amount of polysaccharide repeat unit of the activated saccharide.

Following the click conjugation reaction, unreacted alkyne groups may remain present in the conjugates, these may be capped using a suitable alkyne group capping agent. In one embodiment this alkyne group capping agent is an agent bearing an azido group.

In an embodiment, said alkyne group capping agent is a compound of formula (VI),

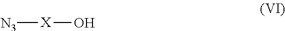

wherein X is (CH₂)ₙ wherein n is selected from 1 to 15.

In one embodiment this alkyne group capping agent is 3-azido-1-propanol.

Therefore, in an embodiment, following step (c) the process further comprises a step of capping the unreacted alkyne groups remained in the conjugates with an alkyne group capping agent.

In an embodiment the capping of the unreacted alkyne groups is performed with an amount of capping agent that is between 0.05 to 20 molar equivalents to the amount of polysaccharide repeat unit of the activated saccharide.

In an embodiment the capping of the unreacted alkyne groups is performed with an amount of capping agent that is between 0.1 to 15 molar equivalents to the amount of polysaccharide repeat unit of the activated saccharide.

In an embodiment the capping of the unreacted alkyne groups is performed with an amount of capping agent that is between 0.5 to 10 molar equivalents to the amount of polysaccharide repeat unit of the activated saccharide.

In an embodiment the capping of the unreacted alkyne groups is performed with an amount of capping agent that is between 0.5 to 5 molar equivalents to the amount of polysaccharide repeat unit of the activated saccharide.

In an embodiment the capping of the unreacted alkyne groups is performed with an amount of capping agent that is between 0.5 to 2 molar equivalents to the amount of polysaccharide repeat unit of the activated saccharide.

In an embodiment the capping of the unreacted alkyne groups is performed with an amount of capping agent that is between 0.5 to 1 molar equivalents to the amount of polysaccharide repeat unit of the activated saccharide.

In an embodiment the capping of the unreacted alkyne groups is performed with an amount of capping agent that is between 1 to 5 molar equivalents to the amount of polysaccharide repeat unit of the activated saccharide.

In an embodiment the capping of the unreacted alkyne groups is performed with an amount of capping agent that is between 1 to 2 molar equivalents to the amount of polysaccharide repeat unit of the activated saccharide.

In an embodiment the capping of the unreacted alkyne groups is performed with an amount of capping agent that is between 1.5 to 2.5 molar equivalents to the amount of polysaccharide repeat unit of the activated saccharide.

In an embodiment the capping of the unreacted alkyne groups is performed with an amount of capping agent that about 0.5 molar equivalent to the amount of polysaccharide repeat unit of the activated saccharide.

In an embodiment the capping of the unreacted alkyne groups is performed with an amount of capping agent that about 1 molar equivalent to the amount of polysaccharide repeat unit of the activated saccharide.

In an embodiment the capping of the unreacted alkyne groups is performed with an amount of capping agent that about 1.5 molar equivalent to the amount of polysaccharide repeat unit of the activated saccharide.

In an embodiment the capping of the unreacted alkyne groups is performed with an amount of capping agent that about 2 molar equivalentS to the amount of polysaccharide repeat unit of the activated saccharide.

In an embodiment the capping of the unreacted alkyne groups is performed with an amount of capping agent that about 2.5 molar equivalents to the amount of polysaccharide repeat unit of the activated saccharide.

In an embodiment the capping of the unreacted alkyne groups is performed with an amount of capping agent that about 5 molar equivalents to the amount of polysaccharide repeat unit of the activated saccharide.

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration. Therefore, in one embodiment the process for producing the glycoconjugate of the present invention comprises the step of purifying the glycoconjugate after it is produced.

In an aspect, the invention provides a glycoconjugate produced according to any of the methods disclosed herein.

In an aspect, the invention provides a capsular saccharide glycoconjugate comprising a capsular saccharide covalently conjugated to a carrier protein (CP) through a spacer and having the general formula (VII):

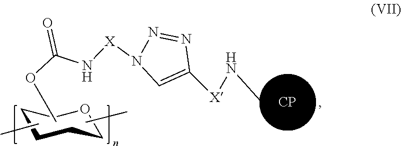

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)ₙ, and O(CH₂CH₂O)ₘCH₂CH₂; where n′ is selected from 1 to 10 and m is selected from 1 to 4,
and wherein X is selected from the group consisting of CH₂O(CH₂)_{n′}CH₂C═O, CH₂O(CH₂CH₂O)_{m′}(CH₂)_{n″}CH₂C═O, where n″ is selected from 0 to 10 and m′ is selected from 0 to 4. Where the structure in square brackets represents a repeat unit of the capsular saccharide.

In an embodiment, the capsular saccharide is not a capsular saccharide from Streptococcus pneumoniae serotype 3.

Formula (VII) is a schematic representation of glycoconjugates of the invention. It should not be understood that a linkage is present at every repeating unit of the saccharide (the structure in square brackets). Rather, a majority of the saccharide repeating unit remains unmodified and covalent linkages between the carrier protein and the saccharide is for a minority of the saccharide repeat units. Additionally, an individual carrier protein (CP) molecule may be linked to more than one saccharide molecule and an individual saccharide molecule can be linked to more than one individual carrier protein (CP) molecule. The structure in square brackets represents a repeat unit of the capsular saccharide.

In a preferred embodiment, the invention provides a glycoconjugate comprising a saccharide covalently conjugated to a carrier protein (CP) through a spacer and having the general formula (VII), wherein X is CH₂(CH₂)_{n′}, where n′ is 2 and wherein X is CH₂O(CH₂)_{n″}CH₂C═O where n″ is 1. In an embodiment, the saccharide is not a capsular saccharide from Streptococcus pneumoniae serotype 3.

Therefore, in a preferred embodiment, the invention provides a capsular saccharide glycoconjugate comprising a capsular saccharide covalently conjugated to a carrier protein (CP) through a spacer and having the general formula (VIII),

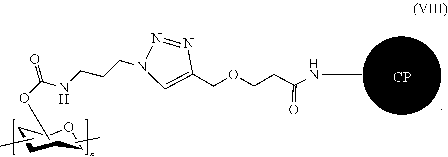

Where the structure in square brackets represents a repeat unit of the capsular saccharide.

Formula (VIII) is a schematic representation of the preferred glycoconjugates of the invention. It should not be understood that a linkage is present at every repeat unit of the saccharide (the structure in square brackets). Rather, a majority of the saccharide repeating unit remains unmodified and covalent linkages between the carrier protein and the saccharide is for a minority of the saccharide repeat units. Additionally, an individual carrier protein (CP) molecule may be linked to more than one saccharide molecule and an individual saccharide molecule can be linked to more than one individual carrier protein (CP) molecule. The structure in square brackets is a schematic representation of a repeat unit of the saccharide.

In an embodiment, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is CH₂(CH₂)_{n′}, where n′ is selected from 1 to 10 and wherein X is CH₂O(CH₂)_{n′}CH₂C═O where n″ is selected from 0 to 10. In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6.

In yet a further embodiment, n′ is 4 and n″ is 6. In yet a further embodiment, n′ is 5 and n″ is 6. In yet a further embodiment, n′ is 6 and n″ is 6. Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is CH₂(CH₂)_{n′}, where n′ is selected from 1 to 10 and wherein CH₂O(CH₂CH₂O)_{m′}(CH₂)_{n′}CH₂C═O, where n″ is selected from 0 to 10 and m′ is selected from 0 to 4.

In an embodiment, n′ is selected from 1 to 5, m′ is selected from 0 to 4 and n″ is selected from 0 to 10. In an embodiment, n′ is selected from 1 to 5, m′ is selected from 0 to 4 and n″ is selected from 0 to 5. In an embodiment, n′ is selected from 1 to 3, m′ is selected from 0 to 2 and n″ is selected from 0 to 3. In an embodiment, n′ is selected from 1 to 2, m′ is selected from 0 to 2 and n″ is selected from 0 to 1.

In a particular embodiment, n′ is 1, m′ is 0 and n″ is 0. In another embodiment, n′ is 1, m′ is 1 and n″ is 0. In another embodiment, n′ is 1, m′ is 2 and n″ is 0. In another embodiment, n′ is 1, m′ is 3 and n″ is 0.

In another embodiment, n′ is 2, m′ is 0 and n″ is 0. In another embodiment, n′ is 2, m′ is 1 and n″ is 0. In another embodiment, n′ is 2, m′ is 2 and n″ is 0. In another embodiment, n′ is 2, m′ is 3 and n″ is 0.

In yet another embodiment, n′ is 3, m′ is 0 and n″ is 0. In yet another embodiment, n′ is 3, m′ is 1 and n″ is 0. In yet another embodiment, n′ is 3, m′ is 2 and n″ is 0. In yet another embodiment, n′ is 3, m′ is 3 and n″ is 0.

In yet a further embodiment, n′ is 4, m′ is 0 and n″ is 0. In yet a further embodiment, n′ is 4, m′ is 1 and n″ is 0. In yet a further embodiment, n′ is 4, m′ is 2 and n″ is 0. In yet a further embodiment, n′ is 4, m′ is 3 and n″ is 0.

In yet a further embodiment, n′ is 5, m′ is 0 and n″ is 0. In yet a further embodiment, n′ is 5, m′ is 1 and n″ is 0. In yet a further embodiment, n′ is 5, m′ is 2 and n″ is 0. In yet a further embodiment, n′ is 5, m′ is 3 and n″ is 0.

In a particular embodiment, n′ is 1, m′ is 0 and n″ is 1. In a particular embodiment, n′ is 1, m′ is 1 and n″ is 1. In a particular embodiment, n′ is 1, m′ is 2 and n″ is 1. In a particular embodiment, n′ is 1, m′ is 3 and n″ is 1.

In another embodiment, n′ is 2, m′ is 0 and n″ is 1. In another embodiment, n′ is 2, m′ is 1 and n″ is 1. In another embodiment, n′ is 2, m′ is 2 and n″ is 1. In another embodiment, n′ is 2, m′ is 3 and n″ is 1.

In yet another embodiment, n′ is 3, m′ is 0 and n″ is 1. In yet another embodiment, n′ is 3, m′ is 1 and n″ is 1. In yet another embodiment, n′ is 3, m′ is 2 and n″ is 1. In yet another embodiment, n′ is 3, m′ is 3 and n″ is 1.

In yet a further embodiment, n′ is 4, m′ is 0 and n″ is 1. In yet a further embodiment, n′ is 4, m′ is 1 and n″ is 1. In yet a further embodiment, n′ is 4, m′ is 2 and n″ is 1. In yet a further embodiment, n′ is 4, m′ is 3 and n″ is 1.

In yet a further embodiment, n′ is 5, m′ is 0 and n″ is 1. In yet a further embodiment, n′ is 5, m′ is 1 and n″ is 1. In yet a further embodiment, n′ is 5, m′ is 2 and n″ is 1. In yet a further embodiment, n′ is 5, m′ is 3 and n″ is 1.

In a particular embodiment, n′ is 1, m′ is 0 and n″ is 2. In a particular embodiment, n′ is 1, m′ is 1 and n″ is 2. In a particular embodiment, n′ is 1, m′ is 2 and n″ is 2. In a particular embodiment, n′ is 1, m′ is 3 and n″ is 2.

In another embodiment, n′ is 2, m′ is 0 and n″ is 2. In another embodiment, n′ is 2, m′ is 1 and n″ is 2. In another embodiment, n′ is 2, m′ is 2 and n″ is 2. In another embodiment, n′ is 2, m′ is 3 and n″ is 2.

In yet another embodiment, n′ is 3, m′ is 0 and n″ is 2. In yet another embodiment, n′ is 3, m′ is 1 and n″ is 2. In yet another embodiment, n′ is 3, m′ is 2 and n″ is 2. In yet another embodiment, n′ is 3, m′ is 3 and n″ is 2.

In yet a further embodiment, n′ is 4, m′ is 0 and n″ is 2. In yet a further embodiment, n′ is 4, m′ is 1 and n″ is 2. In yet a further embodiment, n′ is 4, m′ is 2 and n″ is 2. In yet a further embodiment, n′ is 4, m′ is 3 and n″ is 2.

In yet a further embodiment, n′ is 5, m′ is 0 and n″ is 2. In yet a further embodiment, n′ is 5, m′ is 1 and n″ is 2. In yet a further embodiment, n′ is 5, m′ is 2 and n″ is 2. In yet a further embodiment, n′ is 5, m′ is 3 and n″ is 2.

In a particular embodiment, n′ is 1, m′ is 0 and n″ is 3. In a particular embodiment, n′ is 1, m′ is 1 and n″ is 3. In a particular embodiment, n′ is 1, m′ is 2 and n″ is 3. In a particular embodiment, n′ is 1, m′ is 3 and n″ is 3.

In another embodiment, n′ is 2, m′ is 0 and n″ is 3. In another embodiment, n′ is 2, m′ is 1 and n″ is 3. In another embodiment, n′ is 2, m′ is 2 and n″ is 3. In another embodiment, n′ is 2, m′ is 3 and n″ is 3.

In yet another embodiment, n′ is 3, m′ is 0 and n″ is 3. In yet another embodiment, n′ is 3, m′ is 1 and n″ is 3. In yet another embodiment, n′ is 3, m′ is 2 and n″ is 3. In yet another embodiment, n′ is 3, m′ is 3 and n″ is 3.

In yet a further embodiment, n′ is 4, m′ is 0 and n″ is 3. In yet a further embodiment, n′ is 4, m′ is 1 and n″ is 3. In yet a further embodiment, n′ is 4, m′ is 2 and n″ is 3. In yet a further embodiment, n′ is 4, m′ is 3 and n″ is 3.

In yet a further embodiment, n′ is 5, m′ is 0 and n″ is 3. In yet a further embodiment, n′ is 5, m′ is 1 and n″ is 3. In yet a further embodiment, n′ is 5, m′ is 2 and n″ is 3. In yet a further embodiment, n′ is 5, m′ is 3 and n″ is 3.

In a particular embodiment, n′ is 1, m′ is 0 and n″ is 4. In a particular embodiment, n′ is 1, m′ is 1 and n″ is 4. In a particular embodiment, n′ is 1, m′ is 2 and n″ is 4. In a particular embodiment, n′ is 1, m′ is 3 and n″ is 4.

In another embodiment, n′ is 2, m′ is 0 and n″ is 4. In another embodiment, n′ is 2, m′ is 1 and n″ is 4. In another embodiment, n′ is 2, m′ is 2 and n″ is 4. In another embodiment, n′ is 2, m′ is 3 and n″ is 4.

In yet another embodiment, n′ is 3, m′ is 0 and n″ is 4. In yet another embodiment, n′ is 3, m′ is 1 and n″ is 4. In yet another embodiment, n′ is 3, m′ is 2 and n″ is 4. In yet another embodiment, n′ is 3, m′ is 3 and n″ is 4.

In yet a further embodiment, n′ is 4, m′ is 0 and n″ is 4. In yet a further embodiment, n′ is 4, m′ is 1 and n″ is 4. In yet a further embodiment, n′ is 4, m′ is 2 and n″ is 4. In yet a further embodiment, n′ is 4, m′ is 3 and n″ is 4.

In yet a further embodiment, n′ is 5, m′ is 0 and n″ is 4. In yet a further embodiment, n′ is 5, m′ is 1 and n″ is 4. In yet a further embodiment, n′ is 5, m′ is 2 and n″ is 4. In yet a further embodiment, n′ is 5, m′ is 3 and n″ is 4.

In a particular embodiment, n′ is 1, m′ is 0 and n″ is 5. In a particular embodiment, n′ is 1, m′ is 1 and n″ is 5. In a particular embodiment, n′ is 1, m′ is 2 and n″ is 5. In a particular embodiment, n′ is 1, m′ is 3 and n″ is 5.

In another embodiment, n′ is 2, m′ is 0 and n″ is 5. In another embodiment, n′ is 2, m′ is 1 and n″ is 5. In another embodiment, n′ is 2, m′ is 2 and n″ is 5. In another embodiment, n′ is 2, m′ is 3 and n″ is 5.

In yet another embodiment, n′ is 3, m′ is 0 and n″ is 5. In yet another embodiment, n′ is 3, m′ is 1 and n″ is 5. In yet another embodiment, n′ is 3, m′ is 2 and n″ is 5. In yet another embodiment, n′ is 3, m′ is 3 and n″ is 5.

In yet a further embodiment, n′ is 4, m′ is 0 and n″ is 5. In yet a further embodiment, n′ is 4, m′ is 1 and n″ is 5. In yet a further embodiment, n′ is 4, m′ is 2 and n″ is 5. In yet a further embodiment, n′ is 4, m′ is 3 and n″ is 5.

In yet a further embodiment, n′ is 5, m′ is 0 and n″ is 5. In yet a further embodiment, n′ is 5, m′ is 1 and n″ is 5. In yet a further embodiment, n′ is 5, m′ is 2 and n″ is 5. In yet a further embodiment, n′ is 5, m′ is 3 and n″ is 5.

Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is (CH₂CH₂O)ₘCH₂CH₂, where m is selected from 1 to 4 and wherein X is CH₂O(CH₂)_{n′}CH₂C═O, where n″ is selected from 0 to 10. In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0. In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1.

In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6. Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is (CH₂CH₂O)ₘCH₂CH₂, where m is selected from 1 to 4 and wherein X is CH₂O(CH₂CH₂O)ₘ (CH₂)ₙCH₂C═O, where n″ is selected from 0 to and m′ is selected from 0 to 4.

In an embodiment, m is selected from 1 to 3, m′ is selected from 0 to 4 and n″ is selected from 0 to 10. In an embodiment, m is selected from 1 to 2, m′ is selected from 0 to 4 and n″ is selected from 0 to 5. In an embodiment, m is selected from 1 to 2, m′ is selected from 0 to 2 and n″ is selected from 0 to 3. In an embodiment, m is selected from 1 to 2, m′ is selected from 0 to 2 and n″ is selected from 0 to 1.

In a particular embodiment, m is 1, m′ is 0 and n″ is 0. In another embodiment, m is 1, m′ is 1 and n″ is 0. In another embodiment, m is 1, m′ is 2 and n″ is 0. In another embodiment,
m is 1, m′ is 3 and n″ is 0.

In another embodiment, m is 2, m′ is 0 and n″ is 0. In another embodiment, m is 2, m′ is 1 and n″ is 0. In another embodiment, m is 2, m′ is 2 and n″ is 0. In another embodiment, m is 2, m′ is 3 and n″ is 0.

In yet another embodiment, m is 3, m′ is 0 and n″ is 0. In yet another embodiment, m is 3, m′ is 1 and n″ is 0. In yet another embodiment, m is 3, m′ is 2 and n″ is 0. In yet another embodiment, m is 3, m′ is 3 and n″ is 0.

In yet a further embodiment, m is 4, m′ is 0 and n″ is 0. In yet a further embodiment, m is 4, m′ is 1 and n″ is 0. In yet a further embodiment, m is 4, m′ is 2 and n″ is 0. In yet a further embodiment, m is 4, m′ is 3 and n″ is 0.

In a particular embodiment, m is 1, m′ is 0 and n″ is 1. In a particular embodiment, m is 1, m′ is 1 and n″ is 1. In a particular embodiment, m is 1, m′ is 2 and n″ is 1. In a particular embodiment, m is 1, m′ is 3 and n″ is 1.

In another embodiment, m is 2, m′ is 0 and n″ is 1. In another embodiment, m is 2, m′ is 1 and n″ is 1. In another embodiment, m is 2, m′ is 2 and n″ is 1. In another embodiment, m is 2, m′ is 3 and n″ is 1.

In yet another embodiment, m is 3, m′ is 0 and n″ is 1. In yet another embodiment, m is 3, m′ is 1 and n″ is 1. In yet another embodiment, m is 3, m′ is 2 and n″ is 1. In yet another embodiment, m is 3, m′ is 3 and n″ is 1.

In yet a further embodiment, m is 4, m′ is 0 and n″ is 1. In yet a further embodiment, m is 4, m′ is 1 and n″ is 1. In yet a further embodiment, m is 4, m′ is 2 and n″ is 1. In yet a further embodiment, m is 4, m′ is 3 and n″ is 1.

In a particular embodiment, m is 1, m′ is 0 and n″ is 2. In a particular embodiment, m is 1, m′ is 1 and n″ is 2. In a particular embodiment, m is 1, m′ is 2 and n″ is 2. In a particular embodiment, m is 1, m′ is 3 and n″ is 2.

In another embodiment, m is 2, m′ is 0 and n″ is 2. In another embodiment, m is 2, m′ is 1 and n″ is 2. In another embodiment, m is 2, m′ is 2 and n″ is 2. In another embodiment, m is 2, m′ is 3 and n″ is 2.

In yet another embodiment, m is 3, m′ is 0 and n″ is 2. In yet another embodiment, m is 3, m′ is 1 and n″ is 2. In yet another embodiment, m is 3, m′ is 2 and n″ is 2. In yet another embodiment, m is 3, m′ is 3 and n″ is 2.

In yet a further embodiment, m is 4, m′ is 0 and n″ is 2. In yet a further embodiment, m is 4, m′ is 1 and n″ is 2. In yet a further embodiment, m is 4, m′ is 2 and n″ is 2. In yet a further embodiment, m is 4, m′ is 3 and n″ is 2.

In a particular embodiment, m is 1, m′ is 0 and n″ is 3. In a particular embodiment, m is 1, m′ is 1 and n″ is 3. In a particular embodiment, m is 1, m′ is 2 and n″ is 3. In a particular embodiment, m is 1, m′ is 3 and n″ is 3.

In another embodiment, m is 2, m′ is 0 and n″ is 3. In another embodiment, m is 2, m′ is 1 and n″ is 3. In another embodiment, m is 2, m′ is 2 and n″ is 3. In another embodiment,
m is 2, m′ is 3 and n″ is 3.

In yet another embodiment, m is 3, m′ is 0 and n″ is 3. In yet another embodiment, m is 3, m′ is 1 and n″ is 3. In yet another embodiment, m is 3, m′ is 2 and n″ is 3. In yet another embodiment, m is 3, m′ is 3 and n″ is 3.

In yet a further embodiment, m is 4, m′ is 0 and n″ is 3. In yet a further embodiment, m is 4, m′ is 1 and n″ is 3. In yet a further embodiment, m is 4, m′ is 2 and n″ is 3. In yet a further embodiment, m is 4, m′ is 3 and n″ is 3.

In a particular embodiment, m is 1, m′ is 0 and n″ is 4. In a particular embodiment, m is 1, m′ is 1 and n″ is 4. In a particular embodiment, m is 1, m′ is 2 and n″ is 4. In a particular embodiment, m is 1, m′ is 3 and n″ is 4.

In another embodiment, m is 2, m′ is 0 and n″ is 4. In another embodiment, m is 2, m′ is 1 and n″ is 4. In another embodiment, m is 2, m′ is 2 and n″ is 4. In another embodiment, m is 2, m′ is 3 and n″ is 4.

In yet another embodiment, m is 3, m′ is 0 and n″ is 4. In yet another embodiment, m is 3, m′ is 1 and n″ is 4. In yet another embodiment, m is 3, m′ is 2 and n″ is 4. In yet another embodiment, m is 3, m′ is 3 and n″ is 4.

In yet a further embodiment, m is 4, m′ is 0 and n″ is 4. In yet a further embodiment, m is 4, m′ is 1 and n″ is 4. In yet a further embodiment, m is 4, m′ is 2 and n″ is 4. In yet a further embodiment, m is 4, m′ is 3 and n″ is 4.

In a particular embodiment, m is 1, m′ is 0 and n″ is 5. In a particular embodiment, m is 1, m′ is 1 and n″ is 5. In a particular embodiment, m is 1, m′ is 2 and n″ is 5. In a particular embodiment, m is 1, m′ is 3 and n″ is 5.

In another embodiment, m is 2, m′ is 0 and n″ is 5. In another embodiment, m is 2, m′ is 1 and n″ is 5. In another embodiment, m is 2, m′ is 2 and n″ is 5. In another embodiment, m is 2, m′ is 3 and n″ is 5.

In yet another embodiment, m is 3, m′ is 0 and n″ is 5. In yet another embodiment, m is 3, m′ is 1 and n″ is 5. In yet another embodiment, m is 3, m′ is 2 and n″ is 5. In yet another embodiment, m is 3, m′ is 3 and n″ is 5.

In yet a further embodiment, m is 4, m′ is 0 and n″ is 5. In yet a further embodiment, m is 4, m′ is 1 and n″ is 5. In yet a further embodiment, m is 4, m′ is 2 and n″ is 5. In yet a further embodiment, m is 4, m′ is 3 and n″ is 5.

Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is NHCO(CH₂)_{n′}, where n′ is selected from 1 to 10 and wherein X′ is CH₂O(CH₂)_{n′}CH₂C═O, where n″ is selected from 0 to 10. In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0. In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5. In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6.

In yet a further embodiment, n′ is 4 and n″ is 6. In yet a further embodiment, n′ is 5 and n″ is 6. In yet a further embodiment, n′ is 6 and n″ is 6. Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is NHCO(CH₂)_{n′}, where n′ is selected from 1 to 10 and wherein X is CH₂O(CH₂CH₂O)_{m′}(CH₂)_{n′}CH₂C═O, where n″ is selected from 0 to 10 and m′ is selected from 0 to 4.

In an embodiment, n′ is selected from 1 to 5, m′ is selected from 0 to 4 and n″ is selected from 0 to 10. In an embodiment, n′ is selected from 1 to 5, m′ is selected from 0 to 4 and n″ is selected from 0 to 5. In an embodiment, n′ is selected from 1 to 3, m′ is selected from 0 to 2 and n″ is selected from 0 to 3. In an embodiment, n′ is selected from 1 to 2, m′ is selected from 0 to 2 and n″ is selected from 0 to 1.

In a particular embodiment, n′ is 1, m′ is 0 and n″ is 0. In another embodiment, n′ is 1, m′ is 1 and n″ is 0. In another embodiment, n′ is 1, m′ is 2 and n″ is 0. In another embodiment, n′ is 1, m′ is 3 and n″ is 0.

In another embodiment, n′ is 2, m′ is 0 and n″ is 0. In another embodiment, n′ is 2, m′ is 1 and n″ is 0. In another embodiment, n′ is 2, m′ is 2 and n″ is 0. In another embodiment, n′ is 2, m′ is 3 and n″ is 0.

In yet another embodiment, n′ is 3, m′ is 0 and n″ is 0. In yet another embodiment, n′ is 3, m′ is 1 and n″ is 0. In yet another embodiment, n′ is 3, m′ is 2 and n″ is 0. In yet another embodiment, n′ is 3, m′ is 3 and n″ is 0.

In yet a further embodiment, n′ is 4, m′ is 0 and n″ is 0. In yet a further embodiment, n′ is 4, m′ is 1 and n″ is 0. In yet a further embodiment, n′ is 4, m′ is 2 and n″ is 0. In yet a further embodiment, n′ is 4, m′ is 3 and n″ is 0.

In yet a further embodiment, n′ is 5, m′ is 0 and n″ is 0. In yet a further embodiment, n′ is 5, m′ is 1 and n″ is 0. In yet a further embodiment, n′ is 5, m′ is 2 and n″ is 0. In yet a further embodiment, n′ is 5, m′ is 3 and n″ is 0.

In a particular embodiment, n′ is 1, m′ is 0 and n″ is 1. In a particular embodiment, n′ is 1, m′ is 1 and n″ is 1. In a particular embodiment, n′ is 1, m′ is 2 and n″ is 1. In a particular embodiment, n′ is 1, m′ is 3 and n″ is 1.

In another embodiment, n′ is 2, m′ is 0 and n″ is 1. In another embodiment, n′ is 2, m′ is 1 and n″ is 1. In another embodiment, n′ is 2, m′ is 2 and n″ is 1. In another embodiment, n′ is 2, m′ is 3 and n″ is 1.

In yet another embodiment, n′ is 3, m′ is 0 and n″ is 1. In yet another embodiment, n′ is 3, m′ is 1 and n″ is 1. In yet another embodiment, n′ is 3, m′ is 2 and n″ is 1. In yet another embodiment, n′ is 3, m′ is 3 and n″ is 1.

In yet a further embodiment, n′ is 4, m′ is 0 and n″ is 1. In yet a further embodiment, n′ is 4, m′ is 1 and n″ is 1. In yet a further embodiment, n′ is 4, m′ is 2 and n″ is 1. In yet a further embodiment, n′ is 4, m′ is 3 and n″ is 1.

In yet a further embodiment, n′ is 5, m′ is 0 and n″ is 1. In yet a further embodiment, n′ is 5, m′ is 1 and n″ is 1. In yet a further embodiment, n′ is 5, m′ is 2 and n″ is 1. In yet a further embodiment, n′ is 5, m′ is 3 and n″ is 1.

In a particular embodiment, n′ is 1, m′ is 0 and n″ is 2. In a particular embodiment, n′ is 1, m′ is 1 and n″ is 2. In a particular embodiment, n′ is 1, m′ is 2 and n″ is 2. In a particular embodiment, n′ is 1, m′ is 3 and n″ is 2.

In another embodiment, n′ is 2, m′ is 0 and n″ is 2. In another embodiment, n′ is 2, m′ is 1 and n″ is 2. In another embodiment, n′ is 2, m′ is 2 and n″ is 2. In another embodiment, n′ is 2, m′ is 3 and n″ is 2.

In yet another embodiment, n′ is 3, m′ is 0 and n″ is 2. In yet another embodiment, n′ is 3, m′ is 1 and n″ is 2. In yet another embodiment, n′ is 3, m′ is 2 and n″ is 2. In yet another embodiment, n′ is 3, m′ is 3 and n″ is 2.

In yet a further embodiment, n′ is 4, m′ is 0 and n″ is 2. In yet a further embodiment, n′ is 4, m′ is 1 and n″ is 2. In yet a further embodiment, n′ is 4, m′ is 2 and n″ is 2. In yet a further embodiment, n′ is 4, m′ is 3 and n″ is 2.

In yet a further embodiment, n′ is 5, m′ is 0 and n″ is 2. In yet a further embodiment, n′ is 5, m′ is 1 and n″ is 2. In yet a further embodiment, n′ is 5, m′ is 2 and n″ is 2. In yet a further embodiment, n′ is 5, m′ is 3 and n″ is 2.

In a particular embodiment, n′ is 1, m′ is 0 and n″ is 3. In a particular embodiment, n′ is 1, m′ is 1 and n″ is 3. In a particular embodiment, n′ is 1, m′ is 2 and n″ is 3. In a particular embodiment, n′ is 1, m′ is 3 and n″ is 3.

In another embodiment, n′ is 2, m′ is 0 and n″ is 3. In another embodiment, n′ is 2, m′ is 1 and n″ is 3. In another embodiment, n′ is 2, m′ is 2 and n″ is 3. In another embodiment, n′ is 2, m′ is 3 and n″ is 3.

In yet another embodiment, n′ is 3, m′ is 0 and n″ is 3. In yet another embodiment, n′ is 3, m′ is 1 and n″ is 3. In yet another embodiment, n′ is 3, m′ is 2 and n″ is 3. In yet another embodiment, n′ is 3, m′ is 3 and n″ is 3.

In yet a further embodiment, n′ is 4, m′ is 0 and n″ is 3. In yet a further embodiment, n′ is 4, m′ is 1 and n″ is 3. In yet a further embodiment, n′ is 4, m′ is 2 and n″ is 3. In yet a further embodiment, n′ is 4, m′ is 3 and n″ is 3.

In yet a further embodiment, n′ is 5, m′ is 0 and n″ is 3. In yet a further embodiment, n′ is 5, m′ is 1 and n″ is 3. In yet a further embodiment, n′ is 5, m′ is 2 and n″ is 3. In yet a further embodiment, n′ is 5, m′ is 3 and n″ is 3.

In a particular embodiment, n′ is 1, m′ is 0 and n″ is 4. In a particular embodiment, n′ is 1, m′ is 1 and n″ is 4. In a particular embodiment, n′ is 1, m′ is 2 and n″ is 4. In a particular embodiment, n′ is 1, m′ is 3 and n″ is 4.

In another embodiment, n′ is 2, m′ is 0 and n″ is 4. In another embodiment, n′ is 2, m′ is 1 and n″ is 4. In another embodiment, n′ is 2, m′ is 2 and n″ is 4. In another embodiment, n′ is 2, m′ is 3 and n″ is 4.

In yet another embodiment, n′ is 3, m′ is 0 and n″ is 4. In yet another embodiment, n′ is 3, m′ is 1 and n″ is 4. In yet another embodiment, n′ is 3, m′ is 2 and n″ is 4. In yet another embodiment, n′ is 3, m′ is 3 and n″ is 4.

In yet a further embodiment, n′ is 4, m′ is 0 and n″ is 4. In yet a further embodiment, n′ is 4, m′ is 1 and n″ is 4. In yet a further embodiment, n′ is 4, m′ is 2 and n″ is 4. In yet a further embodiment, n′ is 4, m′ is 3 and n″ is 4.

In yet a further embodiment, n′ is 5, m′ is 0 and n″ is 4. In yet a further embodiment, n′ is 5, m′ is 1 and n″ is 4. In yet a further embodiment, n′ is 5, m′ is 2 and n″ is 4. In yet a further embodiment, n′ is 5, m′ is 3 and n″ is 4.

In a particular embodiment, n′ is 1, m′ is 0 and n″ is 5. In a particular embodiment, n′ is 1, m′ is 1 and n″ is 5. In a particular embodiment, n′ is 1, m′ is 2 and n″ is 5. In a particular embodiment, n′ is 1, m′ is 3 and n″ is 5.

In another embodiment, n′ is 2, m′ is 0 and n″ is 5. In another embodiment, n′ is 2, m′ is 1 and n″ is 5. In another embodiment, n′ is 2, m′ is 2 and n″ is 5. In another embodiment, n′ is 2, m′ is 3 and n″ is 5.

In yet another embodiment, n′ is 3, m′ is 0 and n″ is 5. In yet another embodiment, n′ is 3, m′ is 1 and n″ is 5. In yet another embodiment, n′ is 3, m′ is 2 and n″ is 5. In yet another embodiment, n′ is 3, m′ is 3 and n″ is 5.

In yet a further embodiment, n′ is 4, m′ is 0 and n″ is 5. In yet a further embodiment, n′ is 4, m′ is 1 and n″ is 5. In yet a further embodiment, n′ is 4, m′ is 2 and n″ is 5. In yet a further embodiment, n′ is 4, m′ is 3 and n″ is 5.

In yet a further embodiment, n′ is 5, m′ is 0 and n″ is 5. In yet a further embodiment, n′ is 5, m′ is 1 and n″ is 5. In yet a further embodiment, n′ is 5, m′ is 2 and n″ is 5. In yet a further embodiment, n′ is 5, m′ is 3 and n″ is 5.

Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is NHCO(CH₂CH₂O)ₘCH₂CH₂, where m is selected from 1 to 4 and wherein X is CH₂O(CH₂)_{n′}CH₂C═O, where n″ is selected from 0 to 10. In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0.

In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6. Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is NHCO(CH₂CH₂O)ₘCH₂CH₂, where m is selected from 1 to 4 and wherein X is CH₂O(CH₂CH₂O)_{m′}(CH₂)ₙCH₂C═O, where n″ is selected from 0 to 10 and m′ is selected from 0 to 4.

In an embodiment, m is selected from 1 to 3, m′ is selected from 0 to 4 and n″ is selected from 0 to 10. In an embodiment, m is selected from 1 to 2, m′ is selected from 0 to 4 and n″ is selected from 0 to 5. In an embodiment, m is selected from 1 to 2, m′ is selected from 0 to 2 and n″ is selected from 0 to 3. In an embodiment, m is selected from 1 to 2, m′ is selected from 0 to 2 and n″ is selected from 0 to 1.

In a particular embodiment, m is 1, m′ is 0 and n″ is 0. In another embodiment, m is 1, m′ is 1 and n″ is 0. In another embodiment, m is 1, m′ is 2 and n″ is 0. In another embodiment,
m is 1, m′ is 3 and n″ is 0.

In another embodiment, m is 2, m′ is 0 and n″ is 0. In another embodiment, m is 2, m′ is 1 and n″ is 0. In another embodiment, m is 2, m′ is 2 and n″ is 0. In another embodiment, m is 2, m′ is 3 and n″ is 0.

In yet another embodiment, m is 3, m′ is 0 and n″ is 0. In yet another embodiment, m is 3, m′ is 1 and n″ is 0. In yet another embodiment, m is 3, m′ is 2 and n″ is 0. In yet another embodiment, m is 3, m′ is 3 and n″ is 0.

In yet a further embodiment, m is 4, m′ is 0 and n″ is 0. In yet a further embodiment, m is 4, m′ is 1 and n″ is 0. In yet a further embodiment, m is 4, m′ is 2 and n″ is 0. In yet a further embodiment, m is 4, m′ is 3 and n″ is 0.

In a particular embodiment, m is 1, m′ is 0 and n″ is 1. In a particular embodiment, m is 1, m′ is 1 and n″ is 1. In a particular embodiment, m is 1, m′ is 2 and n″ is 1. In a particular embodiment, m is 1, m′ is 3 and n″ is 1.

In another embodiment, m is 2, m′ is 0 and n″ is 1. In another embodiment, m is 2, m′ is 1 and n″ is 1. In another embodiment, m is 2, m′ is 2 and n″ is 1. In another embodiment, m is 2, m′ is 3 and n″ is 1.

In yet another embodiment, m is 3, m′ is 0 and n″ is 1. In yet another embodiment, m is 3, m′ is 1 and n″ is 1. In yet another embodiment, m is 3, m′ is 2 and n″ is 1. In yet another embodiment, m is 3, m′ is 3 and n″ is 1.

In yet a further embodiment, m is 4, m′ is 0 and n″ is 1. In yet a further embodiment, m is 4, m′ is 1 and n″ is 1. In yet a further embodiment, m is 4, m′ is 2 and n″ is 1. In yet a further embodiment, m is 4, m′ is 3 and n″ is 1.

In a particular embodiment, m is 1, m′ is 0 and n″ is 2. In a particular embodiment, m is 1, m′ is 1 and n″ is 2. In a particular embodiment, m is 1, m′ is 2 and n″ is 2. In a particular embodiment, m is 1, m′ is 3 and n″ is 2.

In another embodiment, m is 2, m′ is 0 and n″ is 2. In another embodiment, m is 2, m′ is 1 and n″ is 2. In another embodiment, m is 2, m′ is 2 and n″ is 2. In another embodiment, m is 2, m′ is 3 and n″ is 2.

In yet another embodiment, m is 3, m′ is 0 and n″ is 2. In yet another embodiment, m is 3, m′ is 1 and n″ is 2. In yet another embodiment, m is 3, m′ is 2 and n″ is 2. In yet another embodiment, m is 3, m′ is 3 and n″ is 2.

In yet a further embodiment, m is 4, m′ is 0 and n″ is 2. In yet a further embodiment, m is 4, m′ is 1 and n″ is 2. In yet a further embodiment, m is 4, m′ is 2 and n″ is 2. In yet a further embodiment, m is 4, m′ is 3 and n″ is 2.

In a particular embodiment, m is 1, m′ is 0 and n″ is 3. In a particular embodiment, m is 1, m′ is 1 and n″ is 3. In a particular embodiment, m is 1, m′ is 2 and n″ is 3. In a particular embodiment, m is 1, m′ is 3 and n″ is 3.

In another embodiment, m is 2, m′ is 0 and n″ is 3. In another embodiment, m is 2, m′ is 1 and n″ is 3. In another embodiment, m is 2, m′ is 2 and n″ is 3. In another embodiment, m is 2, m′ is 3 and n″ is 3.

In yet another embodiment, m is 3, m′ is 0 and n″ is 3. In yet another embodiment, m is 3, m′ is 1 and n″ is 3. In yet another embodiment, m is 3, m′ is 2 and n″ is 3. In yet another embodiment, m is 3, m′ is 3 and n″ is 3.

In yet a further embodiment, m is 4, m′ is 0 and n″ is 3. In yet a further embodiment, m is 4, m′ is 1 and n″ is 3. In yet a further embodiment, m is 4, m′ is 2 and n″ is 3. In yet a further embodiment, m is 4, m′ is 3 and n″ is 3.

In a particular embodiment, m is 1, m′ is 0 and n″ is 4. In a particular embodiment, m is 1, m′ is 1 and n″ is 4. In a particular embodiment, m is 1, m′ is 2 and n″ is 4. In a particular embodiment, m is 1, m′ is 3 and n″ is 4.

In another embodiment, m is 2, m′ is 0 and n″ is 4. In another embodiment, m is 2, m′ is 1 and n″ is 4. In another embodiment, m is 2, m′ is 2 and n″ is 4. In another embodiment, m is 2, m′ is 3 and n″ is 4.

In yet another embodiment, m is 3, m′ is 0 and n″ is 4. In yet another embodiment, m is 3, m′ is 1 and n″ is 4. In yet another embodiment, m is 3, m′ is 2 and n″ is 4. In yet another embodiment, m is 3, m′ is 3 and n″ is 4.

In yet a further embodiment, m is 4, m′ is 0 and n″ is 4. In yet a further embodiment, m is 4, m′ is 1 and n″ is 4. In yet a further embodiment, m is 4, m′ is 2 and n″ is 4. In yet a further embodiment, m is 4, m′ is 3 and n″ is 4.

In a particular embodiment, m is 1, m′ is 0 and n″ is 5. In a particular embodiment, m is 1, m′ is 1 and n″ is 5. In a particular embodiment, m is 1, m′ is 2 and n″ is 5. In a particular embodiment, m is 1, m′ is 3 and n″ is 5.

In another embodiment, m is 2, m′ is 0 and n″ is 5. In another embodiment, m is 2, m′ is 1 and n″ is 5. In another embodiment, m is 2, m′ is 2 and n″ is 5. In another embodiment, m is 2, m′ is 3 and n″ is 5.

In yet another embodiment, m is 3, m′ is 0 and n″ is 5. In yet another embodiment, m is 3, m′ is 1 and n″ is 5. In yet another embodiment, m is 3, m′ is 2 and n″ is 5. In yet another embodiment, m is 3, m′ is 3 and n″ is 5.

In yet a further embodiment, m is 4, m′ is 0 and n″ is 5. In yet a further embodiment, m is 4, m′ is 1 and n″ is 5. In yet a further embodiment, m is 4, m′ is 2 and n″ is 5. In yet a further embodiment, m is 4, m′ is 3 and n″ is 5. Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is OCH₂(CH₂)_{n′}, where n′ is selected from 1 to 10 and wherein X is CH₂O(CH₂)_{n″}CH₂C═O, where n″ is selected from 0 to 10.

In an embodiment, n′ is selected from 1 to 5 and n″ is selected from 0 to 10. In an embodiment, n′ is selected from 1 to 5 and n″ is selected from 0 to 5. In an embodiment, n′ is selected from 1 to 3 and n″ is selected from 0 to 3. In an embodiment, n′ is selected from 1 to 2 and n″ is selected from 0 to 2. In a particular embodiment, n′ is 1 and n″ is 0.

In another embodiment, n′ is 2 and n″ is 0. In yet another embodiment, n′ is 3 and n″ is 0. In yet a further embodiment, n′ is 4 and n″ is 0. In yet a further embodiment, n′ is 5 and n″ is 0. In yet a further embodiment, n′ is 6 and n″ is 0. In a particular embodiment, n′ is 1 and n″ is 1. In another embodiment, n′ is 2 and n″ is 1. In yet another embodiment, n′ is 3 and n″ is 1. In yet a further embodiment, n′ is 4 and n″ is 1. In yet a further embodiment, n′ is 5 and n″ is 1. In yet a further embodiment, n′ is 6 and n″ is 1. In a particular embodiment, n′ is 1 and n″ is 2. In another embodiment, n′ is 2 and n″ is 2. In yet another embodiment, n′ is 3 and n″ is 2. In yet a further embodiment, n′ is 4 and n″ is 2. In yet a further embodiment, n′ is 5 and n″ is 2. In yet a further embodiment, n′ is 6 and n″ is 2. In a particular embodiment, n′ is 1 and n″ is 3. In another embodiment, n′ is 2 and n″ is 3. In yet another embodiment, n′ is 3 and n″ is 3. In yet a further embodiment, n′ is 4 and n″ is 3. In yet a further embodiment, n′ is 5 and n″ is 3. In yet a further embodiment, n′ is 6 and n″ is 3. In a particular embodiment, n′ is 1 and n″ is 4. In another embodiment, n′ is 2 and n″ is 4. In yet another embodiment, n′ is 3 and n″ is 4. In yet a further embodiment, n′ is 4 and n″ is 4. In yet a further embodiment, n′ is 5 and n″ is 4. In yet a further embodiment, n′ is 6 and n″ is 4. In a particular embodiment, n′ is 1 and n″ is 5. In another embodiment, n′ is 2 and n″ is 5. In yet another embodiment, n′ is 3 and n″ is 5.

In yet a further embodiment, n′ is 4 and n″ is 5. In yet a further embodiment, n′ is 5 and n″ is 5. In yet a further embodiment, n′ is 6 and n″ is 5. In a particular embodiment, n′ is 1 and n″ is 6. In another embodiment, n′ is 2 and n″ is 6. In yet another embodiment, n′ is 3 and n″ is 6. In yet a further embodiment, n′ is 4 and n″ is 6. In yet a further embodiment, n′ is 5 and n″ is 6. In yet a further embodiment, n′ is 6 and n″ is 6. Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is OCH₂(CH₂)_{n′}, where n′ is selected from 1 to 10 and wherein X is CH₂O(CH₂CH₂O)_{m′}(CH₂)_{n″}CH₂C═O, where n″ is selected from 0 to 10 and m′ is selected from 0 to 4.

In an embodiment, n′ is selected from 1 to 5, m′ is selected from 0 to 4 and n″ is selected from 0 to 10. In an embodiment, n′ is selected from 1 to 5, m′ is selected from 0 to 4 and n″ is selected from 0 to 5. In an embodiment, n′ is selected from 1 to 3, m′ is selected from 0 to 2 and n″ is selected from 0 to 3. In an embodiment, n′ is selected from 1 to 2, m′ is selected from 0 to 2 and n″ is selected from 0 to 1.

In a particular embodiment, n′ is 1, m′ is 0 and n″ is 0. In another embodiment, n′ is 1, m′ is 1 and n″ is 0. In another embodiment, n′ is 1, m′ is 2 and n″ is 0. In another embodiment, n′ is 1, m′ is 3 and n″ is 0.

In another embodiment, n′ is 2, m′ is 0 and n″ is 0. In another embodiment, n′ is 2, m′ is 1 and n″ is 0. In another embodiment, n′ is 2, m′ is 2 and n″ is 0. In another embodiment, n′ is 2, m′ is 3 and n″ is 0.

In yet another embodiment, n′ is 3, m′ is 0 and n″ is 0. In yet another embodiment, n′ is 3, m′ is 1 and n″ is 0. In yet another embodiment, n′ is 3, m′ is 2 and n″ is 0. In yet another embodiment, n′ is 3, m′ is 3 and n″ is 0.

In yet a further embodiment, n′ is 4, m′ is 0 and n″ is 0. In yet a further embodiment, n′ is 4, m′ is 1 and n″ is 0. In yet a further embodiment, n′ is 4, m′ is 2 and n″ is 0. In yet a further embodiment, n′ is 4, m′ is 3 and n″ is 0.

In yet a further embodiment, n′ is 5, m′ is 0 and n″ is 0. In yet a further embodiment, n′ is 5, m′ is 1 and n″ is 0. In yet a further embodiment, n′ is 5, m′ is 2 and n″ is 0. In yet a further embodiment, n′ is 5, m′ is 3 and n″ is 0.

In a particular embodiment, n′ is 1, m′ is 0 and n″ is 1. In a particular embodiment, n′ is 1, m′ is 1 and n″ is 1. In a particular embodiment, n′ is 1, m′ is 2 and n″ is 1. In a particular embodiment, n′ is 1, m′ is 3 and n″ is 1.

In another embodiment, n′ is 2, m′ is 0 and n″ is 1. In another embodiment, n′ is 2, m′ is 1 and n″ is 1. In another embodiment, n′ is 2, m′ is 2 and n″ is 1. In another embodiment, n′ is 2, m′ is 3 and n″ is 1.

In yet another embodiment, n′ is 3, m′ is 0 and n″ is 1. In yet another embodiment, n′ is 3, m′ is 1 and n″ is 1. In yet another embodiment, n′ is 3, m′ is 2 and n″ is 1. In yet another embodiment, n′ is 3, m′ is 3 and n″ is 1.

In yet a further embodiment, n′ is 4, m′ is 0 and n″ is 1. In yet a further embodiment, n′ is 4, m′ is 1 and n″ is 1. In yet a further embodiment, n′ is 4, m′ is 2 and n″ is 1. In yet a further embodiment, n′ is 4, m′ is 3 and n″ is 1.

In yet a further embodiment, n′ is 5, m′ is 0 and n″ is 1. In yet a further embodiment, n′ is 5, m′ is 1 and n″ is 1. In yet a further embodiment, n′ is 5, m′ is 2 and n″ is 1. In yet a further embodiment, n′ is 5, m′ is 3 and n″ is 1.

In a particular embodiment, n′ is 1, m′ is 0 and n″ is 2. In a particular embodiment, n′ is 1, m′ is 1 and n″ is 2. In a particular embodiment, n′ is 1, m′ is 2 and n″ is 2. In a particular embodiment, n′ is 1, m′ is 3 and n″ is 2.

In another embodiment, n′ is 2, m′ is 0 and n″ is 2. In another embodiment, n′ is 2, m′ is 1 and n″ is 2. In another embodiment, n′ is 2, m′ is 2 and n″ is 2. In another embodiment, n′ is 2, m′ is 3 and n″ is 2.

In yet another embodiment, n′ is 3, m′ is 0 and n″ is 2. In yet another embodiment, n′ is 3, m′ is 1 and n″ is 2. In yet another embodiment, n′ is 3, m′ is 2 and n″ is 2. In yet another embodiment, n′ is 3, m′ is 3 and n″ is 2.

In yet a further embodiment, n′ is 4, m′ is 0 and n″ is 2. In yet a further embodiment, n′ is 4, m′ is 1 and n″ is 2. In yet a further embodiment, n′ is 4, m′ is 2 and n″ is 2. In yet a further embodiment, n′ is 4, m′ is 3 and n″ is 2.

In yet a further embodiment, n′ is 5, m′ is 0 and n″ is 2. In yet a further embodiment, n′ is 5, m′ is 1 and n″ is 2. In yet a further embodiment, n′ is 5, m′ is 2 and n″ is 2. In yet a further embodiment, n′ is 5, m′ is 3 and n″ is 2.

In a particular embodiment, n′ is 1, m′ is 0 and n″ is 3. In a particular embodiment, n′ is 1, m′ is 1 and n″ is 3. In a particular embodiment, n′ is 1, m′ is 2 and n″ is 3. In a particular embodiment, n′ is 1, m′ is 3 and n″ is 3.

In another embodiment, n′ is 2, m′ is 0 and n″ is 3. In another embodiment, n′ is 2, m′ is 1 and n″ is 3. In another embodiment, n′ is 2, m′ is 2 and n″ is 3. In another embodiment, n′ is 2, m′ is 3 and n″ is 3.

In yet another embodiment, n′ is 3, m′ is 0 and n″ is 3. In yet another embodiment, n′ is 3, m′ is 1 and n″ is 3. In yet another embodiment, n′ is 3, m′ is 2 and n″ is 3. In yet another embodiment, n′ is 3, m′ is 3 and n″ is 3.

In yet a further embodiment, n′ is 4, m′ is 0 and n″ is 3. In yet a further embodiment, n′ is 4, m′ is 1 and n″ is 3. In yet a further embodiment, n′ is 4, m′ is 2 and n″ is 3. In yet a further embodiment, n′ is 4, m′ is 3 and n″ is 3.

In yet a further embodiment, n′ is 5, m′ is 0 and n″ is 3. In yet a further embodiment, n′ is 5, m′ is 1 and n″ is 3. In yet a further embodiment, n′ is 5, m′ is 2 and n″ is 3. In yet a further embodiment, n′ is 5, m′ is 3 and n″ is 3.

In a particular embodiment, n′ is 1, m′ is 0 and n″ is 4. In a particular embodiment, n′ is 1, m′ is 1 and n″ is 4. In a particular embodiment, n′ is 1, m′ is 2 and n″ is 4. In a particular embodiment, n′ is 1, m′ is 3 and n″ is 4.

In another embodiment, n′ is 2, m′ is 0 and n″ is 4. In another embodiment, n′ is 2, m′ is 1 and n″ is 4. In another embodiment, n′ is 2, m′ is 2 and n″ is 4. In another embodiment, n′ is 2, m′ is 3 and n″ is 4.

In yet another embodiment, n′ is 3, m′ is 0 and n″ is 4. In yet another embodiment, n′ is 3, m′ is 1 and n″ is 4. In yet another embodiment, n′ is 3, m′ is 2 and n″ is 4. In yet another embodiment, n′ is 3, m′ is 3 and n″ is 4.

In yet a further embodiment, n′ is 4, m′ is 0 and n″ is 4. In yet a further embodiment, n′ is 4, m′ is 1 and n″ is 4. In yet a further embodiment, n′ is 4, m′ is 2 and n″ is 4. In yet a further embodiment, n′ is 4, m′ is 3 and n″ is 4.

In yet a further embodiment, n′ is 5, m′ is 0 and n″ is 4. In yet a further embodiment, n′ is 5, m′ is 1 and n″ is 4. In yet a further embodiment, n′ is 5, m′ is 2 and n″ is 4. In yet a further embodiment, n′ is 5, m′ is 3 and n″ is 4.

In a particular embodiment, n′ is 1, m′ is 0 and n″ is 5. In a particular embodiment, n′ is 1, m′ is 1 and n″ is 5. In a particular embodiment, n′ is 1, m′ is 2 and n″ is 5. In a particular embodiment, n′ is 1, m′ is 3 and n″ is 5.

In another embodiment, n′ is 2, m′ is 0 and n″ is 5. In another embodiment, n′ is 2, m′ is 1 and n″ is 5. In another embodiment, n′ is 2, m′ is 2 and n″ is 5. In another embodiment, n′ is 2, m′ is 3 and n″ is 5.

In yet another embodiment, n′ is 3, m′ is 0 and n″ is 5. In yet another embodiment, n′ is 3, m′ is 1 and n″ is 5. In yet another embodiment, n′ is 3, m′ is 2 and n″ is 5. In yet another embodiment, n′ is 3, m′ is 3 and n″ is 5.

In yet a further embodiment, n′ is 4, m′ is 0 and n″ is 5. In yet a further embodiment, n′ is 4, m′ is 1 and n″ is 5. In yet a further embodiment, n′ is 4, m′ is 2 and n″ is 5. In yet a further embodiment, n′ is 4, m′ is 3 and n″ is 5.

In yet a further embodiment, n′ is 5, m′ is 0 and n″ is 5. In yet a further embodiment, n′ is 5, m′ is 1 and n″ is 5. In yet a further embodiment, n′ is 5, m′ is 2 and n″ is 5. In yet a further embodiment, n′ is 5, m′ is 3 and n″ is 5.

Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is O(CH₂CH₂O)ₘCH₂CH₂, where m is selected from 1 to 4 and wherein X is CH₂O(CH₂)_{n′}CH₂C═O, where n″ is selected from 0 to 10.

In an embodiment, m is selected from 1 to 3 and n″ is selected from 0 to 10. In an embodiment, m is selected from 1 to 3 and n″ is selected from 0 to 5. In an embodiment, m is selected from 1 to 2 and n″ is selected from 0 to 3. In an embodiment, m is selected from 1 to 2 and n″ is selected from 0 to 2. In a particular embodiment, m is 1 and n″ is 0.

In another embodiment, m is 2 and n″ is 0. In yet another embodiment, m is 3 and n″ is 0. In yet a further embodiment, m is 4 and n″ is 0. In a particular embodiment, m is 1 and n″ is 1. In another embodiment, m is 2 and n″ is 1. In yet another embodiment, m is 3 and n″ is 1. In yet a further embodiment, m is 4 and n″ is 1. In a particular embodiment, m is 1 and n″ is 2. In another embodiment, m is 2 and n″ is 2. In yet another embodiment, m is 3 and n″ is 2. In yet a further embodiment, m is 4 and n″ is 2. In a particular embodiment, m is 1 and n″ is 3. In another embodiment, m is 2 and n″ is 3. In yet another embodiment, m is 3 and n″ is 3. In yet a further embodiment, m is 4 and n″ is 3. In a particular embodiment, m is 1 and n″ is 4. In another embodiment, m is 2 and n″ is 4. In yet another embodiment, m is 3 and n″ is 4. In yet a further embodiment, m is 4 and n″ is 4. In a particular embodiment, m is 1 and n″ is 5. In another embodiment, m is 2 and n″ is 5. In yet another embodiment, m is 3 and n″ is 5. In yet a further embodiment, m is 4 and n″ is 5. In a particular embodiment, m is 1 and n″ is 6. In another embodiment, m is 2 and n″ is 6. In yet another embodiment, m is 3 and n″ is 6. In yet a further embodiment, m is 4 and n″ is 6. Preferably, the saccharide is not a capsular saccharide from Streptococcus pneumoniae serotype 3.

In an embodiment, the invention provides a glycoconjugate comprising a saccharide covalently conjugated to a carrier protein (CP) through a spacer and having the general formula (VII), wherein X is O(CH₂CH₂O)ₘCH₂CH₂, where m is selected from 1 to 4 and wherein X is CH₂O(CH₂CH₂O)_{m′}(CH₂)ₙCH₂C═O, where n″ is selected from 0 to 10 and m′ is selected from 0 to 4.

In an embodiment, m is selected from 1 to 3, m′ is selected from 0 to 4 and n″ is selected from 0 to 10. In an embodiment, m is selected from 1 to 2, m′ is selected from 0 to 4 and n″ is selected from 0 to 5. In an embodiment, m is selected from 1 to 2, m′ is selected from 0 to 2 and n″ is selected from 0 to 3. In an embodiment, m is selected from 1 to 2, m′ is selected from 0 to 2 and n″ is selected from 0 to 1.

In a particular embodiment, m is 1, m′ is 0 and n″ is 0. In another embodiment, m is 1, m′ is 1 and n″ is 0. In another embodiment, m is 1, m′ is 2 and n″ is 0. In another embodiment, m is 1, m′ is 3 and n″ is 0.

In another embodiment, m is 2, m′ is 0 and n″ is 0. In another embodiment, m is 2, m′ is 1 and n″ is 0. In another embodiment, m is 2, m′ is 2 and n″ is 0. In another embodiment, m is 2, m′ is 3 and n″ is 0.

In yet another embodiment, m is 3, m′ is 0 and n″ is 0. In yet another embodiment, m is 3, m′ is 1 and n″ is 0. In yet another embodiment, m is 3, m′ is 2 and n″ is 0. In yet another embodiment, m is 3, m′ is 3 and n″ is 0.

In yet a further embodiment, m is 4, m′ is 0 and n″ is 0. In yet a further embodiment, m is 4, m′ is 1 and n″ is 0. In yet a further embodiment, m is 4, m′ is 2 and n″ is 0. In yet a further embodiment, m is 4, m′ is 3 and n″ is 0.

In a particular embodiment, m is 1, m′ is 0 and n″ is 1. In a particular embodiment, m is 1, m′ is 1 and n″ is 1. In a particular embodiment, m is 1, m′ is 2 and n″ is 1. In a particular embodiment, m is 1, m′ is 3 and n″ is 1.

In another embodiment, m is 2, m′ is 0 and n″ is 1. In another embodiment, m is 2, m′ is 1 and n″ is 1. In another embodiment, m is 2, m′ is 2 and n″ is 1. In another embodiment, m is 2, m′ is 3 and n″ is 1.

In yet another embodiment, m is 3, m′ is 0 and n″ is 1. In yet another embodiment, m is 3, m′ is 1 and n″ is 1. In yet another embodiment, m is 3, m′ is 2 and n″ is 1. In yet another embodiment, m is 3, m′ is 3 and n″ is 1.

In yet a further embodiment, m is 4, m′ is 0 and n″ is 1. In yet a further embodiment, m is 4, m′ is 1 and n″ is 1. In yet a further embodiment, m is 4, m′ is 2 and n″ is 1. In yet a further embodiment, m is 4, m′ is 3 and n″ is 1.

In a particular embodiment, m is 1, m′ is 0 and n″ is 2. In a particular embodiment, m is 1, m′ is 1 and n″ is 2. In a particular embodiment, m is 1, m′ is 2 and n″ is 2. In a particular embodiment, m is 1, m′ is 3 and n″ is 2.

In another embodiment, m is 2, m′ is 0 and n″ is 2. In another embodiment, m is 2, m′ is 1 and n″ is 2. In another embodiment, m is 2, m′ is 2 and n″ is 2. In another embodiment, m is 2, m′ is 3 and n″ is 2.

In yet another embodiment, m is 3, m′ is 0 and n″ is 2. In yet another embodiment, m is 3, m′ is 1 and n″ is 2. In yet another embodiment, m is 3, m′ is 2 and n″ is 2. In yet another embodiment, m is 3, m′ is 3 and n″ is 2.

In yet a further embodiment, m is 4, m′ is 0 and n″ is 2. In yet a further embodiment, m is 4, m′ is 1 and n″ is 2. In yet a further embodiment, m is 4, m′ is 2 and n″ is 2. In yet a further embodiment, m is 4, m′ is 3 and n″ is 2.

In a particular embodiment, m is 1, m′ is 0 and n″ is 3. In a particular embodiment, m is 1, m′ is 1 and n″ is 3. In a particular embodiment, m is 1, m′ is 2 and n″ is 3. In a particular embodiment, m is 1, m′ is 3 and n″ is 3.

In another embodiment, m is 2, m′ is 0 and n″ is 3. In another embodiment, m is 2, m′ is 1 and n″ is 3. In another embodiment, m is 2, m′ is 2 and n″ is 3. In another embodiment, m is 2, m′ is 3 and n″ is 3.

In yet another embodiment, m is 3, m′ is 0 and n″ is 3. In yet another embodiment, m is 3, m′ is 1 and n″ is 3. In yet another embodiment, m is 3, m′ is 2 and n″ is 3. In yet another embodiment, m is 3, m′ is 3 and n″ is 3.

In yet a further embodiment, m is 4, m′ is 0 and n″ is 3. In yet a further embodiment, m is 4, m′ is 1 and n″ is 3. In yet a further embodiment, m is 4, m′ is 2 and n″ is 3. In yet a further embodiment, m is 4, m′ is 3 and n″ is 3.

In a particular embodiment, m is 1, m′ is 0 and n″ is 4. In a particular embodiment, m is 1, m′ is 1 and n″ is 4. In a particular embodiment, m is 1, m′ is 2 and n″ is 4. In a particular embodiment, m is 1, m′ is 3 and n″ is 4.

In another embodiment, m is 2, m′ is 0 and n″ is 4. In another embodiment, m is 2, m′ is 1 and n″ is 4. In another embodiment, m is 2, m′ is 2 and n″ is 4. In another embodiment, m is 2, m′ is 3 and n″ is 4.

In yet another embodiment, m is 3, m′ is 0 and n″ is 4. In yet another embodiment, m is 3, m′ is 1 and n″ is 4. In yet another embodiment, m is 3, m′ is 2 and n″ is 4. In yet another embodiment, m is 3, m′ is 3 and n″ is 4.

In yet a further embodiment, m is 4, m′ is 0 and n″ is 4. In yet a further embodiment, m is 4, m′ is 1 and n″ is 4. In yet a further embodiment, m is 4, m′ is 2 and n″ is 4. In yet a further embodiment, m is 4, m′ is 3 and n″ is 4.

In a particular embodiment, m is 1, m′ is 0 and n″ is 5. In a particular embodiment, m is 1, m′ is 1 and n″ is 5. In a particular embodiment, m is 1, m′ is 2 and n″ is 5. In a particular embodiment, m is 1, m′ is 3 and n″ is 5.

In another embodiment, m is 2, m′ is 0 and n″ is 5. In another embodiment, m is 2, m′ is 1 and n″ is 5. In another embodiment, m is 2, m′ is 2 and n″ is 5. In another embodiment, m is 2, m′ is 3 and n″ is 5.

In yet another embodiment, m is 3, m′ is 0 and n″ is 5. In yet another embodiment, m is 3, m′ is 1 and n″ is 5. In yet another embodiment, m is 3, m′ is 2 and n″ is 5. In yet another embodiment, m is 3, m′ is 3 and n″ is 5.

In yet a further embodiment, m is 4, m′ is 0 and n″ is 5. In yet a further embodiment, m is 4, m′ is 1 and n″ is 5. In yet a further embodiment, m is 4, m′ is 2 and n″ is 5. In yet a further embodiment, m is 4, m′ is 3 and n″ is 5.

Preferably, the saccharide is not a capsular saccharide from Streptococcus pneumoniae serotype 3.

Preferably, the saccharide of the glycoconjugate of the presentinvnetion is not a capsular saccharide from Streptococcus pneumoniae serotype 3.

### 1.5 Carrier Protein of the Glycoconjugates of the Invention

A component of the glycoconjugate is a carrier protein to which the saccharide is conjugated. The terms “protein carrier” or “carrier protein” or “carrier” may be used interchangeably herein. Carrier proteins should be amenable to standard conjugation procedures.

In a preferred embodiment, the carrier protein of the glycoconjugate of the invention is selected in the group consisting of: DT (Diphtheria toxoid), TT (tetanus toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa. Another suitable carrier protein is a C5a peptidase from Streptococcus (SCP).

In a preferred embodiment, the carrier protein of the glycoconjugate of the invention is TT, DT, DT mutants (such as CRM₁₉₇) or a C5a peptidase from Streptococcus (SCP).

In an embodiment, the carrier protein of the glycoconjugate of the invention is DT (Diphtheria toxoid). In another embodiment, the carrier protein of the glycoconjugate of the invention is TT (tetanus toxoid).

In another embodiment, the carrier protein of the glycoconjugate of the invention is PD (H. influenzae protein D; see, e.g., EP0594610 B).

In a preferred embodiment, the carrier protein of the glycoconjugate of the invention is CRM₁₉₇ or a C5a peptidase from Streptococcus (SCP).

In a preferred embodiment, the carrier protein of the glycoconjugate of the invention is CRM₁₉₇. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox-} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the carrier protein of the glycoconjugate of the invention is the A chain of CRM₁₉₇ (see CN103495161). In an embodiment, the carrier protein of the glycoconjugate of the invention is the A chain of CRM₁₉₇ obtained via expression by genetically recombinant E. coli (see CN103495161).

In other preferred embodiments, the carrier protein of the glycoconjugate of the invention is SCP (Streptococcal C5a Peptidase). Two important species of β-hemolytic streptococci, Streptococcus pyogenes (group A Streptococcus, GAS) and Streptococcus agalactiae (group B Streptococcus, GBS), which cause a variety of serious human infections that range from mild cases of pharyngitis and impetigo to serious invasive diseases such as necrotizing fasciitis (GAS) and neonatal sepsis (GBS) have developed a way to defeat this immune response. All human isolates of β-hemolytic streptococci, including GAS and GBS, produce a highly conserved cell-wall protein SCP (Streptococcal C5a Peptidase) that specifically inactivates C5a. The scp genes from GAS and GBS encode a polypeptide containing between 1,134 and 1,181 amino acids (Brown et al., PNAS, 2005, vol. 102, no. 51 pages 18391-18396). The first 31 residues are the export signal presequence and are removed upon passing through the cytoplasmic membrane. The next 68 residues serve as a pro-sequence and must be removed to produce active SCP. The next 10 residues can be removed without loss of protease activity. At the other end, starting with Lys-1034, are four consecutive 17-residue motifs followed by a cell sorting and cell-wall attachment signal. This combined signal is composed of a 20-residue hydrophilic sequence containing an LPTTND sequence, a 17-residue hydrophobic sequence, and a short basic carboxyl terminus.

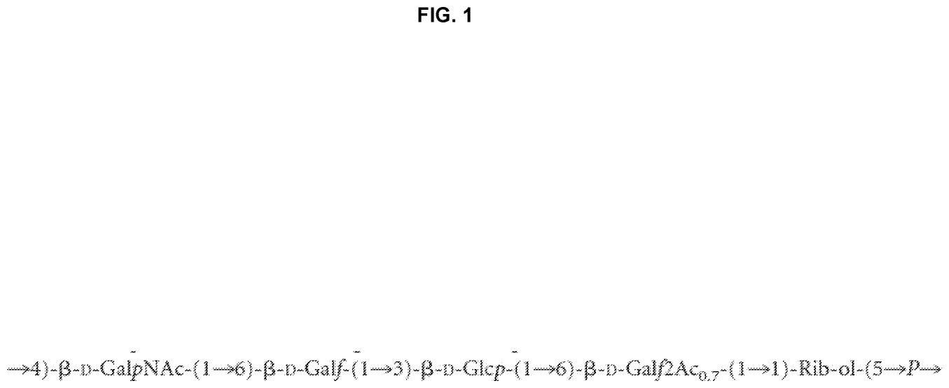

SCP can be divided in domains (see FIG. 1B of Brown et al., PNAS, 2005, vol. 102, no. 51 pages 18391-18396). These domains are the Pre/Pro domain (which comprises the export signal presequence (commonly the first 31 residues) and the pro-sequence (commonly the next 68 residues)), the protease domain (which is splitted in two part (protease part 1 commonly residues 89-333/334 and protease domain part 2 and commonly residues 467/468-583/584), the protease-associated domain (PA domain) (commonly residues 333/334-467/468), three fibronectin type Ill (Fn) domains (Fn1, commonly residues 583/584-712/713; Fn2, commonly residues 712/713-928/929/930; commonly Fn3, residues 929/930-1029/1030/1031) and a cell wall anchor domain (commonly redisues 1029/1030/1031 to the C-terminus).

In an embodiment, the carrier protein of the glycoconjugate of the invention is an SCP from GBS (SCPB). An exemple of SCPB is provided at SEQ ID.NO: 3 of WO97/26008. See also SEQ ID NO: 3 of WO00/34487.

In another preferred embodiments, the carrier protein of the glycoconjugate of the invention is an SCP from GAS (SCPA). Examples of SCPA can be found at SEQ ID.No.1 and SEQ ID.No.2 of WO97/26008. See also SEQ ID NO: 1, 2 and 23 of WO00/34487.

In a preferred embodiment, the carrier protein of the glycoconjugate of the invention is an enzymatically inactive SCP.

In other preferred embodiments, the carrier protein of the glycoconjugate of the invention is an enzymatically inactive SCP from GBS (SCPB).

In another preferred embodiments, the carrier protein of the glycoconjugate of the invention is an enzymatically inactive SCP from GAS (SCPA).

In an embodiment, the carrier protein of the glycoconjugate of the invention is a fragment of an SCP. In an embodiment, the carrier protein of the glycoconjugate of the invention is a fragment of an SCPA. Preferably, the carrier protein of the glycoconjugate of the invention is a fragment of an SCPB.

In an embodiment, the carrier protein of the glycoconjugate of the invention is a fragment of an SCP which comprises the protease domain, the protease-associated domain (PA domain) and the three fibronectin type Ill (Fn) domains but does not comprise the export signal presequence, the pro-sequence and the cell wall anchor domain.

In an embodiment, the carrier protein of the glycoconjugate of the invention is a fragment of an SCP which comprises the protease domain, the protease-associated domain (PA domain) and the three fibronectin type Ill (Fn) domains but does not comprise the export signal presequence, the pro-sequence and the cell wall anchor domain.

In an embodiment, the carrier protein of the glycoconjugate of the invention is an enzymatically inactive fragment of an SCP which comprises the protease domain, the protease-associated domain (PA domain) and two of the three fibronectin type Ill (Fn) domains but does not comprise the export signal presequence, the pro-sequence and the cell wall anchor domain.

In an embodiment, the carrier protein of the glycoconjugate of the invention is an enzymatically inactive fragment of an SCP. In an embodiment, said enzymatically inactive fragment of SCP comprises the protease domain, the protease-associated domain (PA domain) and the three fibronectin type Ill (Fn) domains but does not comprise the export signal presequence, the pro-sequence and the cell wall anchor domain.

In an embodiment, the carrier protein of the glycoconjugate of the invention is an enzymatically inactive fragment of an SCPA. In an embodiment, said enzymatically inactive fragment of an SCPA comprises the protease domain, the protease-associated domain (PA domain) and the three fibronectin type Ill (Fn) domains but does not comprise the export signal presequence, the pro-sequence and the cell wall anchor domain.

In a preferred embodiment, the carrier protein of the glycoconjugate of the invention is an enzymatically inactive fragment of SCPB. Preferably, said enzymatically inactive fragment of SCPB comprises the protease domain, the protease-associated domain (PA domain) and the three fibronectin type Ill (Fn) domains but does not comprise the export signal presequence, the pro-sequence and the cell wall anchor domain.

In an embodiment, the enzymatic activity of SCP is inactivated by replacing at least one amino acid of the wild type sequence. In an embodiment, said replacement is selected from the group consisting of D130A, H193A, N295A and S512A. The numbers indicate the amino acid residue position in the peptidase according to the numbering of SEQ ID NO: 1 of WO00/34487.

Therefore, in an embodiment, the carrier protein of the glycoconjugate of the invention is an enzymatically inactive SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive SCPA where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A.

In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive SCPB where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of an SCP where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of SCP which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of SCPA which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the carrier protein of the glycoconjugate of the invention is an enzymatically inactive fragment of SCPB which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least one amino acid of the wild type sequence. Preferably, said replacement of at least one amino acid is in the protease domain. In an embodiment, said replacement of at least one amino acid is in part 1 of the protease domain. In an embodiment, said replacement of at least one amino acid is in part 2 of the protease domain. In an embodiment, said replacement is selected from the group consisting of D130A, H193A, N295A and S512A. In an embodiment, said replacement is D130A. In another embodiment, said replacement is H193A. In another embodiment, said replacement is N295A. In yet another embodiment, said replacement is S512A.

In an embodiment, the enzymatic activity of SCP is inactivated by replacing at least two amino acids of the wild type sequence. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. In an embodiment, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

Therefore, in an embodiment, the carrier protein of the glycoconjugate of the invention is an enzymatically inactive SCP where said inactivation is accomplished by replacing at least two amino acids of the wild type sequence. Preferably, said replacement of at least two amino acids is in the protease domain. In an embodiment, said replacement of at least two amino acid is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive SCPA where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive SCPB where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of an SCP where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of SCP which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acid is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of SCPA which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least one amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the carrier protein of the glycoconjugate of the invention is an enzymatically inactive fragment of SCPB which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least two amino acids of the wild type sequence. Preferably, said replacement of at least two amino acids is in the protease domain. In an embodiment, said replacement of at least two amino acids is in part 1 of the protease domain. In an embodiment, said replacement of at least two amino acids is in part 2 of the protease domain. In an embodiment, said at least two amino acids replacements are selected from the group consisting of D130A, H193A, N295A and S512A. In an embodiment, said at least two amino acids replacements are D130A and H193A. In an embodiment, said at least two amino acids replacements are D130A and N295A. Preferably, said at least two amino acids replacements are D130A and S512A. In an embodiment, said at least two amino acids replacements are H193A and N295A. In an embodiment, said at least two amino acids replacements are H193A and S512A. In an embodiment, said at least two amino acids replacements are N295A and S512A.

In an embodiment, the enzymatic activity of SCP is inactivated by replacing at least three amino acids of the wild type sequence. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

Therefore, in an embodiment, the carrier protein of the glycoconjugate of the invention is an enzymatically inactive SCP where said inactivation is accomplished by replacing at least three amino acids of the wild type sequence. Preferably, said replacement of at least three amino acids is in the protease domain. In an embodiment, said replacement of at least three amino acid is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive SCPA where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive SCPB where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of an SCP where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of SCP which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acid is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of SCPA which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the carrier protein of the glycoconjugate of the invention is an enzymatically inactive fragment of SCPB which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least three amino acids of the wild type sequence. Preferably, said replacement of at least three amino acids is in the protease domain. In an embodiment, said replacement of at least three amino acids is in part 1 of the protease domain. In an embodiment, said replacement of at least three amino acids is in part 2 of the protease domain. In an embodiment, said at least three amino acids replacements are selected from the group consisting of D130A, H193A, N295A and S512A. In an embodiment, said at least three amino acids replacements are D130A, H193A and N295A. In an embodiment, said at least three amino acids replacements are D130A, H193A and S512A. In an embodiment, said at least three amino acids replacements are D130A, N295A and S512A. In an embodiment, said at least three amino acids replacements are H193A, N295A and S512A.

In an embodiment, the enzymatic activity of SCP is inactivated by replacing at least four amino acids of the wild type sequence. In an embodiment, said at least four amino acids replacements are D130A, H193A, N295A and S512A.

Therefore, in an embodiment, the carrier protein of the glycoconjugate of the invention is an enzymatically inactive SCP where said inactivation is accomplished by replacing at least four amino acids of the wild type sequence. Preferably, said replacement of at least four amino acids is in the protease domain. In an embodiment, said replacement of at least four amino acid is in part 1 of the protease domain. In an embodiment, said replacement of at least four amino acid is in part 2 of the protease domain. In an embodiment, said at least four amino acids replacements are D130A, H193A, N295A and S512A In an embodiment, the carrier protein of the glycoconjugate of the invention is an enzymatically inactive SCPA where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A In an embodiment, the carrier protein of the glycoconjugate of the invention is an enzymatically inactive SCPB where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the glycoconjugate of the invention is an enzymatically inactive fragment of an SCP where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the glycoconjugate of the invention is an enzymatically inactive fragment of SCP which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acid is in part 2 of the protease domain. In an embodiment, said at least four amino acids replacements are D130A, H193A, N295A and S512A

In an embodiment, the carrier protein of the glycoconjugate of the invention is an enzymatically inactive fragment of SCPA which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least one amino acids is in part 2 of the protease domain. In an embodiment, said at least four amino acids replacements are D130A, H193A, N295A and S512A

In an embodiment, the carrier protein of the glycoconjugate of the invention is an enzymatically inactive fragment of SCPB which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least four amino acids of the wild type sequence. Preferably, said replacement of at least four amino acids is in the protease domain. In an embodiment, said replacement of at least four amino acids is in part 1 of the protease domain. In an embodiment, said replacement of at least four amino acids is in part 2 of the protease domain. In an embodiment, said at least four amino acids replacements are D130A, H193A, N295A and S512A

In a particular embodiment, the carrier protein of the glycoconjugate of the invention is an enzymatically inactive fragment of SCP which consists of SEQ ID NO: 41.

In a particular embodiment, the carrier protein of the glycoconjugate of the invention is an enzymatically inactive fragment of SCP which consists of SEQ ID NO: 42.

SEQ ID NO: 41:
MAKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGVVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKWVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYVVEDMAGNITYTPVTKLLEGHSNKPEQ
SEQ ID NO: 41 is 950 amino acids long.
SEQ ID NO: 42:
AKTADTPATSKATIRDLNDPSQVKTLQEKAGKGAGTVVAVIAAGFDKNH
EAWRLTDKAKARYQSKEDLEKAKKEHGITYGEWVNDKVAYYHDYSKDGKT
AVDQEHGTHVSGILSGNAPSETKEPYRLEGAMPEAQLLLMRVEIVNGLAD
YARNYAQAIRDAINLGAKVINMSFGNAALAYANLPDETKKAFDYAKSKGV
SIVTSAGNDSSFGGKTRLPLADHPDYGVVGTPAAADSTLTVASYSPDKQL
TETVTVKTADQQDKEMPVLSTNRFEPNKAYDYAYANRGTKEDDFKDVKGK
IALIERGDIDFKDKIAKAKKAGAVGVLIYDNQDKGFPIELPNVDQMPAAF
ISRKDGLLLKDNPQKTITFNATPKVLPTASGTKLSRFSSWGLTADGNIKP
DIAAPGQDILSSVANNKYAKLSGTAMSAPLVAGIMGLLQEQYETQYPDMT
PSERLDLAKKVLMSSATALYDEDEKAYFSPRQQGAGAVDAKKASAATMYV
TDKDNTSSKVHLNNVSDKFEVTVTVHNKSDKPQELYYQATVQTDKVDGKH
FALAPKALYETSWQKITIPANSSKQVTVPIDASRFSKDLLAQMKNGYFLE
GFVRFKQDPKKEELMSIPYIGFRGDFGNLSALEKPIYDSKDGSSYYHEAN
SDAKDQLDGDGLQFYALKNNFTALTTESNPWTIIKAVKEGVENIEDIESS
EITETIFAGTFAKQDDDSHYYIHRHANGKPYAAISPNGDGNRDYVQFQGT
FLRNAKNLVAEVLDKEGNVVWTSEVTEQVVKNYNNDLASTLGSTRFEKTR
WDGKDKDGKVVANGTYTYRVRYTPISSGAKEQHTDFDVIVDNTTPEVATS
ATFSTEDRRLTLASKPKTSQPVYRERIAYTYMDEDLPTTEYISPNEDGTF
TLPEEAETMEGATVPLKMSDFTYVVEDMAGNITYTPVTKLLEGHSNKPEQ
SEQ ID NO: 42 is 949 amino acids long.

In a particular embodiment, the carrier protein of the glycoconjugate of the invention is an enzymatically inactive fragment of SCP consisting of a polypeptide having at least 90% identity with SEQ ID NO: 41.

In a particular embodiment, the carrier protein of the glycoconjugate of the invention is an enzymatically inactive fragment of SCP consisting of a polypeptide having at least 95% identity with SEQ ID NO: 41.

In a particular embodiment, the carrier protein of the glycoconjugate of the invention is an enzymatically inactive fragment of SCP consisting of a polypeptide having at least 99% identity with SEQ ID NO: 41.

In a particular embodiment, the carrier protein of the glycoconjugate of the invention is an enzymatically inactive fragment of SCP consisting of a polypeptide having at least 99.5% identity with SEQ ID NO: 41.

In a particular embodiment, the carrier protein of the glycoconjugate of the invention is an enzymatically inactive fragment of SCP consisting of a polypeptide having at least 99.8% identity with SEQ ID NO: 41.

In a particular embodiment, the carrier protein of the glycoconjugate of the invention is an enzymatically inactive fragment of SCP consisting of a polypeptide having at least 99.85% identity with SEQ ID NO: 41.

In a particular embodiment, the carrier protein of the glycoconjugate of the invention is an enzymatically inactive fragment of SCP consisting of a polypeptide having at least 90% identity with SEQ ID NO: 42.

In a particular embodiment, the carrier protein of the glycoconjugate of the invention is an enzymatically inactive fragment of SCP consisting of a polypeptide having at least 95% identity with SEQ ID NO: 42.

In a particular embodiment, the carrier protein of the glycoconjugate of the invention is an enzymatically inactive fragment of SCP consisting of a polypeptide having at least 99% identity with SEQ ID NO: 42.

In a particular embodiment, the carrier protein of the glycoconjugate of the invention is an enzymatically inactive fragment of SCP consisting of a polypeptide having at least 99.5% identity with SEQ ID NO: 42.

In a particular embodiment, the carrier protein of the glycoconjugate of the invention is an enzymatically inactive fragment of SCP consisting of a polypeptide having at least 99.8% identity with SEQ ID NO: 42.

In a particular embodiment, the carrier protein of the glycoconjugate of the invention is an enzymatically inactive fragment of SCP consisting of a polypeptide having at least 99.85% identity with SEQ ID NO: 42.

### 2 Immunogenic Compositions

### 2.1 Combinations of Glycoconjugates of the Invention

In an embodiment the invention relates to an immunogenic composition comprising a glycoconjugate of the invention (as disclosed at section 1 above).

In an embodiment the invention relates to an immunogenic composition comprising a glycoconjugate of the invention and comprising from 1 to 25 different glycoconjugates.

In an embodiment the invention relates to an immunogenic composition comprising a glycoconjugate of the invention and comprising from 1 to 25 glycoconjugates from different serotypes of S. pneumoniae (1 to 25 pneumococcal conjugates). In one embodiment the invention relates to an immunogenic composition comprising glycoconjugates from 7, 8, 9, 10, 11, 12, 13, 14, 15, 16, 17, 18, 19, 20, 21, 22, 23, 24 or different serotypes of S. pneumoniae. In one embodiment the immunogenic composition comprises glycoconjugates from 16 or 20 different serotypes of S. pneumoniae. In an embodiment the immunogenic composition is a 7, 8, 9, 10, 11, 12, 13, 14, 15, 16, 17, 18, 19 or 20-valent pneumococcal conjugate compositions. In an embodiment the immunogenic composition is a 14, 15, 16, 17, 18 or 19-valent pneumococcal conjugate compositions. In an embodiment the immunogenic composition is a 16-valent pneumococcal conjugate composition. In an embodiment the immunogenic composition is a 19-valent pneumococcal conjugate composition. In an embodiment the immunogenic composition is a 20-valent pneumococcal conjugate composition.

In an embodiment the immunogenic composition is a 21, 22, 23, 24 or 25-valent pneumococcal conjugate compositions. In an embodiment the immunogenic composition is a 21-valent pneumococcal conjugate composition. In an embodiment the immunogenic composition is a 22-valent pneumococcal conjugate composition. In an embodiment the immunogenic composition is a 23-valent pneumococcal conjugate composition. In an embodiment the immunogenic composition is a 24-valent pneumococcal conjugate composition. In an embodiment the immunogenic composition is a 25-valent pneumococcal conjugate composition.

In an embodiment the invention relates to an immunogenic composition comprising a glycoconjugate of the invention and comprising glycoconjugates from S. pneumoniae serotypes 1, 3, 4, 5, 6B, 7F, 9V, 14, 18C, 19F and 23F. In an embodiment the immunogenic composition is an 11-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 9V, 14, 18C, 19A, 19F and 23F. In an embodiment the immunogenic composition is a 13-valent pneumococcal conjugate compositions.

In an embodiment the invention relates to an immunogenic composition comprising a glycoconjugate of the invention and further comprising glycoconjugates from S. pneumoniae serotypes 1, 3, 4, 5, 6A, 6B, 7F, 9V, 14, 18C, 19A, 19F, 22F, 23F and 33F.

In an embodiment the immunogenic composition is a 15-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F and 33F. In an embodiment the immunogenic composition is a 20-valent pneumococcal conjugate compositions.

In an embodiment the invention relates to an immunogenic composition comprising a glycoconjugate of the invention and further comprising glycoconjugates from S. pneumoniae serotypes 1, 2, 3, 4, 5, 6A, 6B, 7F, 8, 9V, 10A, 11A, 12F, 14, 15B, 18C, 19A, 19F, 22F, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 9N, 10A, 11A, 12F, 14, 15B, 18C, 19A, 19F, 22F, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 2, 4, 5, 6A, 6B, 7F, 8, 9V, 9N, 10A, 11A, 12F, 14, 15B, 18C, 19A, 19F, 22F, 23F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 2, 4, 5, 6A, 6B, 7F, 8, 9V, 9N, 10A, 11A, 12F, 14, 15B, 17F, 18C, 19A, 19F, 22F, 23F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 2, 3, 4, 5, 6A, 6B, 7F, 8, 9V, 9N, 10A, 11A, 12F, 14, 15B, 17F, 18C, 19A, 19F, 20, 22F, 23F and 33F. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F and 33F. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 33F and 35B. In an embodiment the immunogenic composition is a 21-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F and 33F. In an embodiment the immunogenic composition is a 22-valent pneumococcal conjugate compositions.

In an embodiment the invention relates to an immunogenic composition comprising a glycoconjugate of the invention and further comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 24F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 33F and 35B. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 24F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 33F and 35B. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 24F and 33F. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 33F and 35B. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23F, 24F, 33F and 35B. In an embodiment the immunogenic composition is a 22-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 24F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 24F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23F, 24F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 24F and 33F. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23F, 24F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23B, 23F, 24F, 33F and 35B. In an embodiment the immunogenic composition is a 23-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F and 33F. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 33F and 35B. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23F, 24F, 33F and 35B. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23B, 23F, 24F, 33F and 35B. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15B, 18C, 19A, 19F, 22F, 23A, 23B, 23F, 24F, 33F and 35B. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 1, 3, 4, 5, 6A, 6B, 7F, 8, 9V, 10A, 11A, 12F, 14, 15A, 15B, 18C, 19A, 19F, 22F, 23A, 23B, 23F, 24F, 33F and 35B. In an embodiment the immunogenic composition is a 25-valent pneumococcal conjugate compositions.

In an embodiment the invention relates to an immunogenic composition comprising a glycoconjugate of the invention selected from the group consisting of glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38.

In an embodiment the invention relates to an immunogenic composition comprising a glycoconjugate of the invention selected from the group consisting of glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38. In an embodiment the immunogenic composition is a 21-valent pneumococcal conjugate compositions.

In an embodiment the invention relates to an immunogenic composition comprising a glycoconjugate of the invention and comprising twenty one glycoconjugates selected from the group consisting of glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38. In an embodiment the immunogenic composition is a 22-valent pneumococcal conjugate compositions.

In an embodiment the invention relates to an immunogenic composition comprising a glycoconjugate of the invention and comprising at least one glycoconjugate selected from the group consisting of glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38.

In an embodiment the invention relates to an immunogenic composition comprising a glycoconjugate of the invention and comprising twenty two glycoconjugates selected from the group consisting of glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38. In an embodiment the immunogenic composition is a 23-valent pneumococcal conjugate compositions.

In an embodiment the invention relates to an immunogenic composition comprising a glycoconjugate of the invention and comprising twenty three glycoconjugates selected from the group consisting of glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38. In an embodiment the immunogenic composition is a 24-valent pneumococcal conjugate compositions.

In an embodiment the invention relates to an immunogenic composition comprising a glycoconjugate of the invention and comprising glycoconjugates from S. pneumoniae serotypes 2, 9N, 15A, 17F, 20, 23A, 23B, 24F and 35B. In an embodiment the immunogenic composition is a 10-valent pneumococcal conjugate compositions.

In an embodiment the invention relates to an immunogenic composition comprising a glycoconjugate of the invention and comprising glycoconjugates from S. pneumoniae serotypes 2, 9N, 15A, 17F, 19A, 19F, 20, 23A, 23B, 24F and 35B. In an embodiment the immunogenic composition is a 12-valent pneumococcal conjugate compositions.

In an embodiment the invention relates to an immunogenic composition comprising a glycoconjugate of the invention and comprising glycoconjugates from S. pneumoniae serotypes 2, 7C, 9N, 10B, 15A, 16F, 17F, 20, 21, 22A, 23A, 23B, 24B, 24F, 27, 29, 31, 33B, 34, 35B, 35F and 38. In an embodiment the immunogenic composition is a 23-valent pneumococcal conjugate compositions.

In an embodiment the invention relates to an immunogenic composition comprising a glycoconjugate of the invention and omprising glycoconjugates from S. pneumoniae serotypes 2, 7C, 9N, 10B, 15A, 16F, 17F, 19A, 19F, 20, 21, 22A, 23A, 23B, 24B, 24F, 27, 29, 31, 33B, 34, 35B, 35F and 38. In an embodiment the immunogenic composition is a 25-valent pneumococcal conjugate compositions.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugate of the invention is conjugated to CRM₁₉₇. In an embodiment of any of the above immunogenic compositions, the glycoconjugate of the invention is conjugated to SCP.

In an embodiment of any of the above immunogenic compositions, the glycoconjugate from S. pneumoniae serotype 22F is conjugated to CRM₁₉₇. In an embodiment of any of the above immunogenic compositions, the glycoconjugate from S. pneumoniae serotype 33F is conjugated to CRM₁₉₇. In an embodiment of any of the above immunogenic compositions, the glycoconjugate from S. pneumoniae serotype 15B is conjugated to CRM₁₉₇. In an embodiment of any of the above immunogenic compositions, the glycoconjugate from S. pneumoniae serotype 12F is conjugated to CRM₁₉₇. In an embodiment of any of the above immunogenic compositions, the glycoconjugate from S. pneumoniae serotype 10A is conjugated to CRM₁₉₇. In an embodiment of any of the above immunogenic compositions, the glycoconjugate from S. pneumoniae serotype 11A is conjugated to CRM₁₉₇. In an embodiment of any of the above immunogenic compositions, the glycoconjugate from S. pneumoniae serotype 8 is conjugated to CRM₁₉₇. In an embodiment of any of the above immunogenic compositions, the glycoconjugates from S. pneumoniae serotypes 4, 6B, 9V, 14, 18C, 19F and 23F are conjugated to CRM₁₉₇. In an embodiment of any of the above immunogenic compositions, the glycoconjugates from S. pneumoniae serotypes 1, 5 and 7F are conjugated to CRM₁₉₇. In an embodiment of any of the above immunogenic compositions, the glycoconjugates from S. pneumoniae serotypes 6A and 19A are conjugated to CRM₁₉₇.

In an embodiment of any of the above immunogenic compositions, the glycoconjugates of any of the above immunogenic compositions are all individually conjugated to CRM₁₉₇.

In an embodiment of any of the above immunogenic compositions, the glycoconjugate of the invention is conjugated to SCP and the other glycoconjugate(s) is/are all individually conjugated to CRM₁₉₇.

In an embodiment, the glycoconjugates from S. pneumoniae serotypes 1, 4, 5, 6B, 7F, 9V, 14 and/or 23F of any of the above immunogenic compositions are individually conjugated to PD.

In an embodiment, the glycoconjugate from S. pneumoniae serotype 18C of any of the above immunogenic compositions is conjugated to TT.

In an embodiment, the glycoconjugate from S. pneumoniae serotype 19F of any of the above immunogenic compositions is conjugated to DT.

In an embodiment, the glycoconjugates from S. pneumoniae serotypes 1, 4, 5, 6B, 7F, 9V, 14 and/or 23F of any of the above immunogenic compositions are individually conjugated to PD, the glycoconjugate from S. pneumoniae serotype 18C is conjugated to TT and the glycoconjugate from S. pneumoniae serotype 19F is conjugated to DT.

In an embodiment the above immunogenic compositions comprise from 8 to 25 different serotypes of S. pneumoniae.

Compositions of the invention may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

### 2.2 Dosage of the Immunogenic Compositions of the Invention

The amount of glycoconjugate(s) in each dose is selected as an amount which induces an immunoprotective response without significant, adverse side effects in typical vaccinees. Such amount will vary depending upon which specific immunogen is employed and how it is presented.

The amount of a particular glycoconjugate in an immunogenic composition can be calculated based on total saccharide for that conjugate (conjugated and non-conjugated). For example, a glycoconjugate with 20% free saccharide will have about 80 μg of conjugated saccharide and about 20 μg of nonconjugated saccharide in a 100 μg saccharide dose. The amount of glycoconjugate can vary depending upon the bacteria and bacteria serotype. The saccharide concentration can be determined by the uronic acid assay.

The “immunogenic amount” of the different saccharide components in the immunogenic composition, may diverge and each may comprise about 0.5 μg, about 0.75 μg, about 1 μg, about 2 μg, about 3 μg, about 4 μg, about 5 μg, about 6 μg, about 7 μg, about 8 μg, about 9 μg, about 10 μg, about 15 μg, about 20 μg, about 30 μg, about 40 μg, about 50 μg, about 60 μg, about 70 μg, about 80 μg, about 90 μg, or about 100 μg of any particular saccharide antigen.

Generally, each dose will comprise 0.1 μg to 100 μg of saccharide. In an embodiment each dose will comprise 0.1 μg to 100 μg of saccharide. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of serotype 3 polysaccharide. Any whole number integer within any of the above ranges is contemplated as an embodiment of the disclosure.

In an embodiment, each dose will comprise about 0.5 μg of saccharide. In an embodiment, each dose will comprise about 0.55 μg of saccharide. In an embodiment, each dose will comprise about 0.75 μg of saccharide. In an embodiment, each dose will comprise about 1.0 μg of saccharide. In an embodiment, each dose will comprise about 1.1 μg of saccharide. In an embodiment, each dose will comprise about 1.5 μg of saccharide. In an embodiment, each dose will comprise about 2.0 μg of saccharide. In an embodiment, each dose will comprise about 2.2 μg of saccharide. In an embodiment, each dose will comprise about 2.5 μg of saccharide. In an embodiment, each dose will comprise about 3.0 μg of saccharide. In an embodiment, each dose will comprise about 3.5 μg of saccharide. In an embodiment, each dose will comprise about 4.0 μg of saccharide. In an embodiment, each dose will comprise about 4.4 μg of saccharide. In an embodiment, each dose will comprise about 5.0 μg of saccharide. In an embodiment, each dose will comprise about 5.5 μg of saccharide. In an embodiment, each dose will comprise about 6.0 μg of saccharide.

Generally, each dose will comprise 0.1 μg to 100 μg of saccharide for a given bacteria or serotype. In an embodiment each dose will comprise 0.1 μg to 100 μg of saccharide for a given bacteria or serotype. In a preferred embodiment each dose will comprise 0.5 μg to 20 μg. In a preferred embodiment each dose will comprise 1.0 μg to 10 μg. In an even preferred embodiment, each dose will comprise 2.0 μg to 5.0 μg of accharide for a given bacteria or serotype. Any whole number integer within any of the above ranges is contemplated as an embodiment of the disclosure.

In an embodiment, each dose will comprise about 0.5 μg of saccharide for each particular glycoconjugate. In an embodiment, each dose will comprise about 0.55 μg of saccharide for each particular glycoconjugate. In an embodiment, each dose will comprise about 0.75 μg of saccharide for each particular glycoconjugate. In an embodiment, each dose will comprise about 1.0 μg of saccharide for each particular glycoconjugate. In an embodiment, each dose will comprise about 1.1 μg of saccharide for each particular glycoconjugate. In an embodiment, each dose will comprise about 1.5 μg of saccharide for each particular glycoconjugate. In an embodiment, each dose will comprise about 2.0 μg of saccharide for each particular glycoconjugate. In an embodiment, each dose will comprise about 2.2 μg of saccharide for each particular glycoconjugate. In an embodiment, each dose will comprise about 2.5 μg of saccharide for each particular glycoconjugate. In an embodiment, each dose will comprise about 3.0 μg of saccharide for each particular glycoconjugate. In an embodiment, each dose will comprise about 3.5 μg of saccharide for each particular glycoconjugate. In an embodiment, each dose will comprise about 4.0 μg of saccharide for each particular glycoconjugate. In an embodiment, each dose will comprise about 4.4 μg of saccharide for each particular glycoconjugate. In an embodiment, each dose will comprise about 5.0 μg of saccharide for each particular glycoconjugate. In an embodiment, each dose will comprise about 5.5 μg of saccharide for each particular glycoconjugate. In an embodiment, each dose will comprise about 6.0 μg of saccharide for each particular glycoconjugate.

### 2.3 Carrier Amount

Generally, each dose will comprise 10 μg to 150 μg of carrier protein, particularly 15 μg to 100 μg of carrier protein, more particularly 25 μg to 75 μg of carrier protein, and even more particularly 40 μg to 60 μg of carrier protein. In an embodiment, said carrier protein is CRM₁₉₇. In an embodiment, said carrier protein is SCP.

In an embodiment, each dose will comprise about 10 μg, about 15 μg, about 20 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg or about 75 μg of carrier protein.

In an embodiment, each dose will comprise about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 51 μg, about 52 μg, about 53 μg, about 54 μg, about 55 μg, about 56 μg, about 57 μg, about 58 μg, about 59 μg, about 60 μg, about 61 μg, about 62 μg, about 63 μg, about 64 μg, about 65 μg, about 66 μg, about 67 μg, about 68 μg, about 69 μg, about 70 μg, about 71 μg, about 72 μg, about 73 μg, about 74 μg or about 75 μg of carrier protein.

In an embodiment, each dose will comprise about 30 μg of carrier protein. In an embodiment, each dose will comprise about 31 μg of carrier protein. In an embodiment, each dose will comprise about 32 μg of carrier protein. In an embodiment, each dose will comprise about 33 μg of carrier protein. In an embodiment, each dose will comprise about 34 μg of carrier protein. In an embodiment, each dose will comprise about 45 μg of carrier protein.

In an embodiment, each dose will comprise about 40 μg of carrier protein. In an embodiment, each dose will comprise about 41 μg of carrier protein. In an embodiment, each dose will comprise about 42 μg of carrier protein. In an embodiment, each dose will comprise about 43 μg of carrier protein. In an embodiment, each dose will comprise about 44 μg of carrier protein. In an embodiment, each dose will comprise about 45 μg of carrier protein.

In an embodiment, each dose will comprise about 48 μg of carrier protein. In an embodiment, each dose will comprise about 49 μg of carrier protein. In an embodiment, each dose will comprise about 50 μg of carrier protein. In an embodiment, each dose will comprise about 51 μg of carrier protein. In an embodiment, each dose will comprise about 52 μg of carrier protein. In an embodiment, each dose will comprise about 53 μg of carrier protein.

In an embodiment, said carrier protein is CRM₁₉₇.

In an embodiment, said carrier protein is SCP.

### 2.4 Further Antigens

Immunogenic compositions of the invention comprise conjugated saccharide antigen(s) (glycoconjugate(s)). They may also further include antigen(s) from other pathogen(s), particularly from bacteria and/or viruses. Preferred further antigens are selected from: a diphtheria toxoid (D), a tetanus toxoid (T), a pertussis antigen (P), which is typically acellular (Pa), a hepatitis B virus (HBV) surface antigen (HBsAg), a hepatitis A virus (HAV) antigen, a conjugated Haemophilus influenzae type b capsular saccharide (Hib), inactivated poliovirus vaccine (IPV).

In an embodiment, the immunogenic compositions of the invention comprise D-T-Pa. In an embodiment, the immunogenic compositions of the invention comprise D-T-Pa-Hib, D-T-Pa-IPV or D-T-Pa-HBsAg. In an embodiment, the immunogenic compositions of the invention comprise D-T-Pa-HBsAg-IPV or D-T-Pa-HBsAg-Hib. In an embodiment, the immunogenic compositions of the invention comprise D-T-Pa-HBsAg-IPV-Hib.

Pertussis antigens: Bordetella pertussis causes whooping cough. Pertussis antigens in vaccines are either cellular (whole cell, in the form of inactivated B. pertussis cells) or acellular. Preparation of cellular pertussis antigens is well documented (e.g., it may be obtained by heat inactivation of phase I culture of B. pertussis). Preferably, however, the invention uses acellular antigens. Where acellular antigens are used, it is preferred to use one, two or (preferably) three of the following antigens: (1) detoxified pertussis toxin (pertussis toxoid, or PT); (2) filamentous hemagglutinin (FHA); (3) pertactin (also known as the 69 kilodalton outer membrane protein). FHA and pertactin may be treated with formaldehyde prior to use according to the invention. PT is preferably detoxified by treatment with formaldehyde and/or glutaraldehyde. Acellular pertussis antigens are preferably adsorbed onto one or more aluminum salt adjuvants. As an alternative, they may be added in an unadsorbed state. Where pertactin is added then it is preferably already adsorbed onto an aluminum hydroxide adjuvant. PT and FHA may be adsorbed onto an aluminum hydroxide adjuvant or an aluminum phosphate. Adsorption of all of PT, FHA and pertactin to aluminum hydroxide is most preferred.

Inactivated poliovirus vaccine: Poliovirus causes poliomyelitis. Rather than use oral poliovirus vaccine, preferred embodiments of the invention use IPV. Prior to administration to patients, polioviruses must be inactivated, and this can be achieved by treatment with formaldehyde. Poliomyelitis can be caused by one of three types of poliovirus. The three types are similar and cause identical symptoms, but they are antigenically different and infection by one type does not protect against infection by others. It is therefore preferred to use three poliovirus antigens in the invention: poliovirus Type 1 (e.g., Mahoney strain), poliovirus Type 2 (e.g., MEF-1 strain), and poliovirus Type 3 (e.g., Saukett strain). The viruses are preferably grown, purified and inactivated individually, and are then combined to give a bulk trivalent mixture for use with the invention.

Diphtheria toxoid: Corynebacterium diphtheriae causes diphtheria. Diphtheria toxin can be treated (e.g., using formalin or formaldehyde) to remove toxicity while retaining the ability to induce specific anti-toxin antibodies after injection. These diphtheria toxoids are used in diphtheria vaccines. Preferred diphtheria toxoids are those prepared by formaldehyde treatment. The diphtheria toxoid can be obtained by growing C. diphtheriae in growth medium, followed by formaldehyde treatment, ultrafiltration and precipitation. The toxoided material may then be treated by a process comprising sterile filtration and/or dialysis. The diphtheria toxoid is preferably adsorbed onto an aluminum hydroxide adjuvant.

Tetanus toxoid: Clostridium tetani causes tetanus. Tetanus toxin can be treated to give a protective toxoid. The toxoids are used in tetanus vaccines. Preferred tetanus toxoids are those prepared by formaldehyde treatment. The tetanus toxoid can be obtained by growing C. tetani in growth medium, followed by formaldehyde treatment, ultrafiltration and precipitation. The material may then be treated by a process comprising sterile filtration and/or dialysis.

Hepatitis A virus antigens: Hepatitis A virus (HAV) is one of the known agents which causes viral hepatitis. A preferred HAV component is based on inactivated virus, and inactivation can be achieved by formalin treatment.

Hepatitis B virus (HBV) is one of the known agents which causes viral hepatitis. The major component of the capsid is a protein known as HBV surface antigen or, more commonly, HBsAg, which is typically a 226-amino acid polypeptide with a molecular weight of −24 kDa. All existing hepatitis B vaccines contain HBsAg, and when this antigen is administered to a normal vaccinee it stimulates the production of anti-HBsAg antibodies which protect against HBV infection.

For vaccine manufacture, HBsAg has been made in two ways: purification of the antigen in particulate form from the plasma of chronic hepatitis B carriers or expression of the protein by recombinant DNA methods (e.g., recombinant expression in yeast cells). Unlike native HBsAg (i.e., as in the plasma-purified product), yeast-expressed HBsAg is generally non-glycosylated, and this is the most preferred form of HBsAg for use with the invention.

Conjugated Haemophilus influenzae type b antigens: Haemophilus influenzae type b (Hib) causes bacterial meningitis. Hib vaccines are typically based on the capsular saccharide antigen, the preparation of which is well documented. The Hib saccharide can be conjugated to a carrier protein in order to enhance its immunogenicity, especially in children. Typical carrier proteins are tetanus toxoid, diphtheria toxoid, CRM₁₉₇, H. influenzae protein D, and an outer membrane protein complex from serogroup B meningococcus. The saccharide moiety of the conjugate may comprise full-length polyribosylribitol phosphate (PRP) as prepared from Hib bacteria, and/or fragments of full-length PRP. Hib conjugates may or may not be adsorbed to an aluminum salt adjuvant.

### 2.5 Adjuvant(s)

In some embodiments, the immunogenic compositions disclosed herein may further comprise at least one, two or three adjuvants. In some embodiments, the immunogenic compositions disclosed herein may further comprise at least one adjuvant. In some embodiments, the immunogenic compositions disclosed herein may further comprise one adjuvant. In some embodiments, the immunogenic compositions disclosed herein may further comprise two adjuvants. The term “adjuvant” refers to a compound or mixture that enhances the immune response to an antigen. Antigens may act primarily as a delivery system, primarily as an immune modulator or have strong features of both.

Suitable adjuvants include those suitable for use in mammals, including humans. Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant. In a preferred embodiment, the immunogenic compositions disclosed herein comprise aluminum phosphate as adjuvant.

Further exemplary adjuvants to enhance effectiveness of the immunogenic compositions as disclosed herein include, but are not limited to: (1) oil-in-water emulsion formulations (with or without other specific immunostimulating agents such as muramyl peptides (see below) or bacterial cell wall components), such as for example (a) SAF, containing 10% Squalene, 0.4% Tween 80, 5% pluronic-blocked polymer L121, and thr-MDP either microfluidized into a submicron emulsion or vortexed to generate a larger particle size emulsion, and (b) RIBI™ adjuvant system (RAS), (Ribi Immunochem, Hamilton, MT) containing 2% Squalene, 0.2% Tween 80, and one or more bacterial cell wall components such as monophosphorylipid A (MPL), trehalose dimycolate (TDM), and cell wall skeleton (CWS), preferably MPL+CWS (DETOX™); (2) saponin adjuvants, such as QS21, STIMULON™ (Cambridge Bioscience, Worcester, MA), ABISCO@(Isconova, Sweden), or ISCOMATRIX@(Commonwealth Serum Laboratories, Australia), may be used or particles generated therefrom such as ISCOMs (immunostimulating complexes), which ISCOMS may be devoid of additional detergent (e.g., WO 00/07621); (3) Complete Freund's Adjuvant (CFA) and Incomplete Freund's Adjuvant (IFA); (4) cytokines, such as interleukins (e.g., IL-1, IL-2, IL-4, IL-5, IL-6, IL-7, IL-12 (e.g., WO 99/44636)), interferons (e.g., gamma interferon), macrophage colony stimulating factor (M-CSF), tumor necrosis factor (TNF), etc.; (5) monophosphoryl lipid A (MPL) or 3-O-deacylated MPL (3dMPL) (see, e.g., GB-2220221, EP0689454), optionally in the substantial absence of alum when used with pneumococcal saccharides (see, e.g., WO 00/56358); (6) combinations of 3dMPL with, for example, QS21 and/or oil-in-water emulsions (see, e.g., EP0835318, EP0735898, EP0761231); (7) a polyoxyethylene ether or a polyoxyethylene ester (see, e.g., WO 99/52549); (8) a polyoxyethylene sorbitan ester surfactant in combination with an octoxynol (e.g., WO 01/21207) or a polyoxyethylene alkyl ether or ester surfactant in combination with at least one additional non-ionic surfactant such as an octoxynol (e.g., WO 01/21152); (9) a saponin and an immunostimulatory oligonucleotide (e.g., a CpG oligonucleotide) (e.g., WO 00/62800); (10) an immunostimulant and a particle of metal salt (see, e.g., WO 00/23105); (11) a saponin and an oil-in-water emulsion (e.g., WO 99/11241); (12) a saponin (e.g., QS21)+3dMPL+IM2 (optionally+a sterol) (e.g., WO 98/57659); (13) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc.

In an embodiment of the present invention, the immunogenic compositions as disclosed herein comprise a CpG Oligonucleotide as adjuvant. A CpG oligonucleotide as used herein refers to an immunostimulatory CpG oligodeoxynucleotide (CpG ODN), and accordingly these terms are used interchangeably unless otherwise indicated. Immunostimulatory CpG oligodeoxynucleotides contain one or more immunostimulatory CpG motifs that are unmethylated cytosine-guanine dinucleotides, optionally within certain preferred base contexts. The methylation status of the CpG immunostimulatory motif generally refers to the cytosine residue in the dinucleotide. An immunostimulatory oligonucleotide containing at least one unmethylated CpG dinucleotide is an oligonucleotide which contains a 5′ unmethylated cytosine linked by a phosphate bond to a 3′ guanine, and which activates the immune system through binding to Toll-like receptor 9 (TLR-9). In another embodiment the immunostimulatory oligonucleotide may contain one or more methylated CpG dinucleotides, which will activate the immune system through TLR9 but not as strongly as if the CpG motif(s) was/were unmethylated. CpG immunostimulatory oligonucleotides may comprise one or more palindromes that in turn may encompass the CpG dinucleotide. CpG oligonucleotides have been described in a number of issued patents, published patent applications, and other publications, including U.S. Pat. Nos. 6,194,388; 6,207,646; 6,214,806; 6,218,371; 6,239,116; and 6,339,068. In an embodiment of the present invention, the immunogenic compositions as disclosed herein comprise any of the CpG Oligonucleotide described at page 3, line 22, to page 12, line 36, of WO 2010/125480.

Different classes of CpG immunostimulatory oligonucleotides have been identified. These are referred to as A, B, C and P class, and are described in greater detail at page 3, line 22, to page 12, line 36, of WO 2010/125480. Methods of the invention embrace the use of these different classes of CpG immunostimulatory oligonucleotides.

In an embodiment of the present invention, the immunogenic compositions as disclosed herein comprise an A class CpG oligonucleotide. Preferably, the “A class” CpG oligonucleotide of the invention has the following nucleic acid sequence: 5′ GGGGACGACGTCGTGGGGGGG 3′ (SEQ ID NO: 1). Some non-limiting examples of A-Class oligonucleotides include: 5′ G*G*G_G_A_C_G_A_C_G_T_C_G_T_G_G*G*G*G*G*G 3′ (SEQ ID NO: 2); wherein refers to a phosphorothioate bond and “_” refers to a phosphodiester bond.

In an embodiment of the present invention, the immunogenic compositions as disclosed herein comprise a B class CpG Oligonucleotide. In one embodiment, the CpG oligonucleotide for use in the present invention is a B class CpG oligonucleotide represented by at least the formula: 5′ X₁X₂CGX3X₄ 3′, wherein X1, X2, X3, and X4 are nucleotides. In one embodiment, X₂ is adenine, guanine, or thymine. In another embodiment, X₃ is cytosine, adenine, or thymine.

The B class CpG oligonucleotide sequences of the invention are those broadly described above as well as disclosed in WO 96/02555, WO 98/18810 and U.S. Pat. Nos. 6,194,388; 6,207,646; 6,214,806; 6,218,371; 6,239,116 and 6,339,068. Exemplary sequences include but are not limited to those disclosed in these latter applications and patents.

In an embodiment, the “B class” CpG oligonucleotide of the invention has the following nucleic acid sequence:

(SEQ ID NO: 3)
5′ TCGTCGTTTTTCGGTGCTTTT 3′,
or
(SEQ ID NO: 4)
5′ TCGTCGTTTTTCGGTCGTTTT 3′,
or
(SEQ ID NO: 5)
5′ TCGTCGTTTTGTCGTTTTGTCGTT 3′,
or
(SEQ ID NO: 6)
5′ TCGTCGTTTCGTCGTTTTGTCGTT 3′,
or
(SEQ ID NO: 7)
5′ TCGTCGTTTTGTCGTTTTTTTCGA 3′

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

Some non-limiting examples of B-Class oligonucleotides include:

(SEQ ID NO: 8)
5′ T*C*G*T*C*G*T*T*T*T*T*C*G*G*T*G*C*T*T*T*T 3′,
or
(SEQ ID NO: 9)
5′ T*C*G*T*C*G*T*T*T*T*T*C*G*G*T*C*G*T*T*T*T 3′,
or
(SEQ ID NO: 10)
5′ T*C*G*T*C*G*T*T*T*T*G*T*C*G*T*T*T*T*G*
T*C*G*T*T 3′,
or
(SEQ ID NO: 11)
5′ T*C*G*T*C*G*T*T*T*C*G*T*C*G*T*T*T*T*G*
T*C*G*T*T 3′,
or
(SEQ ID NO: 12)
5′ T*C*G*T*C*G*T*T*T*T*G*T*C*G*T*T*T*T*T*
T*T*C*G*A 3′.

wherein “*” refers to a phosphorothioate bond.

In an embodiment of the present invention, the immunogenic compositions as disclosed herein comprise a C class CpG Oligonucleotide. In an embodiment, the “C class” CpG oligonucleotides of the invention have the following nucleic acid sequence:

(SEQ ID NO: 13)
5′ TCGCGTCGTTCGGCGCGCGCCG 3′,
or
(SEQ ID NO: 14)
5′ TCGTCGACGTTCGGCGCGCGCCG 3′,
or
(SEQ ID NO: 15)
5′ TCGGACGTTCGGCGCGCGCCG 3′,
or
(SEQ ID NO: 16)
5′ TCGGACGTTCGGCGCGCCG 3′,
or
(SEQ ID NO: 17)
5′ TCGCGTCGTTCGGCGCGCCG 3′,
or
(SEQ ID NO: 18)
5′ TCGACGTTCGGCGCGCGCCG 3′,
or
(SEQ ID NO: 19)
5′ TCGACGTTCGGCGCGCCG 3′,
or
(SEQ ID NO: 20)
5′ TCGCGTCGTTCGGCGCCG 3′,
or
(SEQ ID NO: 21)
5′ TCGCGACGTTCGGCGCGCGCCG 3′,
or
(SEQ ID NO: 22)
5′ TCGTCGTTTTCGGCGCGCGCCG 3′,
or
(SEQ ID NO: 23)
5′ TCGTCGTTTTCGGCGGCCGCCG 3′,
or
(SEQ ID NO: 24)
5′ TCGTCGTTTTACGGCGCCGTGCCG 3′,
or
(SEQ ID NO: 25)
5′ TCGTCGTTTTCGGCGCGCGCCGT 3′

In any of these sequences, all of the linkages may be all phosphorothioate bonds. In another embodiment, in any of these sequences, one or more of the linkages may be phosphodiester, preferably between the “C” and the “G” of the CpG motif making a semi-soft CpG oligonucleotide.

Some non-limiting examples of C-Class oligonucleotides include:

(SEQ ID NO: 26)
5′ T*C_G*C_G*T*C_G*T*T*C_G*G*C*G*C_G*C*G*C*C*G 3′,
or
(SEQ ID NO: 27)
5′ T*C_G*T*C_G*A*C_G*T*T*C_G*G*C*G*C_G*C*G*C*C*G 3′,
or
(SEQ ID NO: 28)
5′ T*C_G*G*A*C_G*T*T*C_G*G*C*G*C_G*C*G*C*C*G 3′,
or
(SEQ ID NO: 29)
5′ T*C_G*G*A*C_G*T*T*C_G*G*C*G*C*G*C*C*G 3′,
or
(SEQ ID NO: 30)
5′ T*C_G*C_G*T*C_G*T*T*C_G*G*C*G*C*G*C*C*G 3′,
or
(SEQ ID NO: 31)
5′ T*C_G*A*C_G*T*T*C_G*G*C*G*C_G*C*G*C*C*G 3′,
or
(SEQ ID NO: 32)
5′ T*C_G*A*C_G*T*T*C_G*G*C*G*C*G*C*C*G 3′,
or
(SEQ ID NO: 33)
5′ T*C_G*C_G*T*C_G*T*T*C_G*G*C*G*C*C*G 3′,
or
(SEQ ID NO: 34)
5′ T*C_G*C_G*A*C_G*T*T*C_G*G*C*G*C_G*C*G*C*C*G 3′,
or
(SEQ ID NO: 35)
5′ T*C*G*T*C*G*T*T*T*T*C*G*G*C*G*C*G*C*G*C*C*G 3′,
or
(SEQ ID NO: 36)
5′ T*C*G*T*C*G*T*T*T*T*C*G*G*C*G*G*C*C*G*C*C*G 3′,
or

(SEQ ID NO: 37)
5′ T*C*G*T*C_G*T*T*T*T*A*C_G*G*C*G*C*C_G*
T*G*C*C*G 3′,
or
(SEQ ID NO: 38)
5′ T*C_G*T*C*G*T*T*T*T*C*G*G*C*G*C*G*C*G*
C*C*G*T 3′

wherein “*” refers to a phosphorothioate bond and “_” refers to a phosphodiester bond. In any of these sequences, an ethyl-uridine or a halogen may substitute for the 5′ T; examples of halogen substitutions include but are not limited to bromo-uridine or iodo-uridine substitutions.

In an embodiment of the present invention, the immunogenic compositions as disclosed herein comprise a P class CpG Oligonucleotide. In an embodiment, the CpG oligonucleotide for use in the present invention is a P class CpG oligonucleotide containing a 5′ TLR activation domain and at least two palindromic regions, one palindromic region being a 5′ palindromic region of at least 6 nucleotides in length and connected to a 3′ palindromic region of at least 8 nucleotides in length either directly or through a spacer, wherein the oligonucleotide includes at least one YpR dinucleotide. In an embodiment, said oligonucleotide is not T*C_G*T*C_G*A*C_G*T*T*C_G*G*C*G*C_G*C*G*C*C*G (SEQ ID NO: 27). In one embodiment the P class CpG oligonucleotide includes at least one unmethylated CpG dinucleotide. In another embodiment the TLR activation domain is TCG, TTCG, TTTCG, TYpR, TTYpR, TTTYpR, UCG, UUCG, UUUCG, TTT, or TTTT. In yet another embodiment the TLR activation domain is within the 5′ palindromic region. In another embodiment the TLR activation domain is immediately 5′ to the 5′ palindromic region. In an embodiment, the “P class” CpG oligonucleotides of the invention have the following nucleic acid sequence: 5′ TCGTCGACGATCGGCGCGCGCCG 3′ (SEQ ID NO: 39).

In said sequences, all of the linkages may be all phosphorothioate bonds. In another embodiment, one or more of the linkages may be phosphodiester, preferably between the “C” and the “G” of the CpG motif making a semi-soft CpG oligonucleotide. In any of these sequences, an ethyl-uridine or a halogen may substitute for the 5′ T; examples of halogen substitutions include but are not limited to bromo-uridine or iodo-uridine substitutions.

A non-limiting example of P-Class oligonucleotides include:

(SEQ ID NO: 40)
5′ T*C_G*T*C_G*A*C_G*A*T*C_G*G*C*G*C_G*
C*G*C*C*G 3′

wherein “*” refers to a phosphorothioate bond and “_” refers to a phosphodiester bond. In one embodiment the oligonucleotide includes at least one phosphorothioate linkage. In another embodiment all internucleotide linkages of the oligonucleotide are phosphorothioate linkages. In another embodiment the oligonucleotide includes at least one phosphodiester-like linkage. In another embodiment the phosphodiester-like linkage is a phosphodiester linkage. In another embodiment a lipophilic group is conjugated to the oligonucleotide. In one embodiment the lipophilic group is cholesterol.

In an embodiment, all the internucleotide linkages of the CpG oligonucleotides disclosed herein are phosphodiester bonds (“soft” oligonucleotides, as described in WO 2007/026190). In another embodiment, CpG oligonucleotides of the invention are rendered resistant to degradation (e.g., are stabilized). A “stabilized oligonucleotide” refers to an oligonucleotide that is relatively resistant to in vivo degradation (e.g., via an exo- or endo-nuclease). Nucleic acid stabilization can be accomplished via backbone modifications. Oligonucleotides having phosphorothioate linkages provide maximal activity and protect the oligonucleotide from degradation by intracellular exo- and endo-nucleases.

The immunostimulatory oligonucleotides may have a chimeric backbone, which have combinations of phosphodiester and phosphorothioate linkages. For purposes of the instant invention, a chimeric backbone refers to a partially stabilized backbone, wherein at least one internucleotide linkage is phosphodiester or phosphodiester-like, and wherein at least one other internucleotide linkage is a stabilized internucleotide linkage, wherein the at least one phosphodiester or phosphodiester-like linkage and the at least one stabilized linkage are different. When the phosphodiester linkage is preferentially located within the CpG motif such molecules are called “semi-soft” as described in WO 2007/026190.

Other modified oligonucleotides include combinations of phosphodiester, phosphorothioate, methylphosphonate, methylphosphorothioate, phosphorodithioate, and/or p-ethoxy linkages.

Mixed backbone modified ODN may be synthesized as described in WO 2007/026190.

The size of the CpG oligonucleotide (i.e., the number of nucleotide residues along the length of the oligonucleotide) also may contribute to the stimulatory activity of the oligonucleotide. For facilitating uptake into cells, CpG oligonucleotide of the invention preferably have a minimum length of 6 nucleotide residues. Oligonucleotides of any size greater than 6 nucleotides (even many kb long) are capable of inducing an immune response if sufficient immunostimulatory motifs are present, because larger oligonucleotides are degraded inside cells. In certain embodiments, the CpG oligonucleotides are 6 to 100 nucleotides long, preferentially 8 to 30 nucleotides long. In important embodiments, nucleic acids and oligonucleotides of the invention are not plasmids or expression vectors.

In an embodiment, the CpG oligonucleotide disclosed herein comprise substitutions or modifications, such as in the bases and/or sugars as described at paragraphs 134 to 147 of WO 2007/026190.

In an embodiment, the CpG oligonucleotide of the present invention is chemically modified. Examples of chemical modifications are known to the skilled person and are described, for example in Uhlmann et al. (1990) Chem. Rev. 90:543; S. Agrawal, Ed., Humana Press, Totowa, USA 1993; Crooke et al. (1996) Annu. Rev. Pharmacol. Toxicol. 36:107-129; and Hunziker et al. (1995) Mod. Synth. Methods 7:331-417. An oligonucleotide according to the invention may have one or more modifications, wherein each modification is located at a particular phosphodiester internucleoside bridge and/or at a particular R-D-ribose unit and/or at a particular natural nucleoside base position in comparison to an oligonucleotide of the same sequence which is composed of natural DNA or RNA.

In some embodiments of the invention, CpG-containing nucleic acids might be simply mixed with immunogenic carriers according to methods known to those skilled in the art (see, e.g., WO 03/024480).

In a particular embodiment of the present invention, any of the immunogenic compositions disclosed herein comprise from 2 μg to 100 mg of CpG oligonucleotide. In a particular embodiment of the present invention, the immunogenic composition of the invention comprises 0.1 mg to 50 mg of CpG oligonucleotide, preferably from 0.2 mg to 10 mg CpG oligonucleotide, more preferably from 0.3 mg to 5 mg CpG oligonucleotide.

In a particular embodiment of the present invention, the immunogenic composition of the invention comprises from 0.3 mg to 5 mg CpG oligonucleotide. Even preferably, the immunogenic composition of the invention may comprise from 0.5 to 2 mg CpG oligonucleotide. Most preferably, the immunogenic composition of the invention may comprise from 0.75 to 1.5 mg CpG oligonucleotide. In a preferred embodiment, any of the immunogenic composition disclosed herein may comprise about 1 mg CpG oligonucleotide.

### 3 Formulation

The immunogenic compositions of the invention may be formulated in liquid form (i.e., solutions or suspensions) or in a lyophilized form. In an embodiment, the immunogenic composition of the invention is formulated in a liquid form. In an embodiment, the immunogenic composition of the invention is formulated in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of glycoconjugates disclosed herein and a pharmaceutically acceptable excipient, carrier, or diluent.

In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer. In an embodiment, said buffer has a pKa of about 3.5 to about 7.5. In some embodiments, the buffer is phosphate, succinate, histidine or citrate. In some embodiments, the buffer is succinate. In some embodiments, the buffer is histidine. In certain embodiments, the buffer is succinate at a final concentration of 1 mM to 10 mM. In one particular embodiment, the final concentration of the succinate buffer is about 5 mM.

In an embodiment, the immunogenic compositions of the disclosure comprise a salt. In some embodiments, the salt is selected from the groups consisting of magnesium chloride, potassium chloride, sodium chloride and a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer.

In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w).

In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 0.02% polysorbate 20 (w/w). In another embodiment, the final concentration of the polysorbate 20 in the formulation is 0.01% polysorbate 20 (w/w). In another embodiment, the final concentration of the polysorbate 20 in the formulation is 0.03% polysorbate 20 (w/w). In another embodiment, the final concentration of the polysorbate 20 in the formulation is 0.04% polysorbate 80 (w/w). In another embodiment, the final concentration of the polysorbate 20 in the formulation is 0.05% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the disclosure has a pH of 5.5 to 7.5, more preferably a pH of 5.6 to 7.0, even more preferably a pH of 5.8 to 6.0.

In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL. In an embodiment, the immunogenic composition of the invention for injection has a volume of 0.2 mL to 1 mL, even more preferably a volume of about 0.5 mL.

### 4 Uses of the Glycoconjugate and Immunogenic Compositions of the Invention

The glycoconjugates disclosed herein may be use as antigens. For example, they may be part of a vaccine.

Therefore, in an embodiment, the immunogenic compositions of the invention are for use as a medicament.

In an embodiment, the immunogenic compositions of the invention are for use as a vaccine.

Therefore, in an embodiment, the immunogenic compositions described herein are for use in generating an immune response in a subject. In one aspect, the subject is a mammal, such as a human, non-human primate, cat, sheep, pig, horse, bovine or dog. In one aspect, the subject is a human.

The immunogenic compositions described herein may be used in therapeutic or prophylactic methods for preventing, treating or ameliorating a bacterial infection, disease or condition in a subject. Thus, in one aspect, the disclosure provides a method of preventing, treating or ameliorating an infection, disease or condition associated with a bacterial infection in a subject, comprising administering to the subject an immunologically effective amount of an immunogenic composition of the disclosure.

The immunogenic composition of the present disclosure can be used to protect or treat a human susceptible to a bacterial infection, by means of administering the immunogenic composition via a systemic or mucosal route. In an embodiment, the immunogenic composition of the invention is administered by intramuscular, intraperitoneal, intradermal or subcutaneous routes. In an embodiment, the immunogenic composition of the invention is administered by intramuscular, intraperitoneal, intradermal or subcutaneous injection. In an embodiment, the immunogenic composition of the invention is administered by intramuscular or subcutaneous injection. In an embodiment, the immunogenic composition of the invention is administered by intramuscular injection. In an embodiment, the immunogenic composition of the invention is administered by subcutaneous injection.

5. Subject to be Treated with the Immunogenic Compositions of the Invention

As disclosed herein, the immunogenic compositions described herein may be used in various therapeutic or prophylactic methods for preventing, treating or ameliorating a bacterial infection, disease or condition in a subject.

In a preferred embodiment, said subject is a human. In a most preferred embodiment, said subject is a newborn (i.e., under three months of age), an infant (i.e., from 3 months to one year of age) or a toddler (i.e., from one year to four years of age).

In an embodiment, the immunogenic compositions disclosed herein are for use as a vaccine.

In such embodiment, the subject to be vaccinated may be less than 1 year of age. For example, the subject to be vaccinated can be about 1, about 2, about 3, about 4, about 5, about 6, about 7, about 8, about 9, about 10, about 11 or about 12 months of age. In an embodiment, the subject to be vaccinated is about 2, about 4 or about 6 months of age. In another embodiment, the subject to be vaccinated is less than 2 years of age. For example, the subject to be vaccinated can be about 12 to about 15 months of age. In some cases, as little as one dose of the immunogenic composition according to the invention is needed, but under some circumstances, a second, third or fourth dose may be given (see section 8 below).

In an embodiment of the present invention, the subject to be vaccinated is a human adult 50 years of age or older, more preferably a human adult 55 years of age or older. In an embodiment, the subject to be vaccinated is a human adult 65 years of age or older, 70 years of age or older, 75 years of age or older or 80 years of age or older.

In an embodiment the subject to be vaccinated is an immunocompromised individual, in particular a human. An immunocompromised individual is generally defined as a person who exhibits an attenuated or reduced ability to mount a normal humoral or cellular defense to challenge by infectious agents.

In an embodiment of the present invention, the immunocompromised subject to be vaccinated suffers from a disease or condition that impairs the immune system and results in an antibody response that is insufficient to protect against or treat pneumococcal disease.

In an embodiment, said disease is a primary immunodeficiency disorder. Preferably, said primary immunodeficiency disorder is selected from the group consisting of: combined T- and B-cell immunodeficiencies, antibody deficiencies, well-defined syndromes, immune dysregulation diseases, phagocyte disorders, innate immunity deficiencies, autoinflammatory disorders, and complement deficiencies. In an embodiment, said primary immunodeficiency disorder is selected from the one disclosed on page 24, line 11, to page 25, line 19, of WO 2010/125480.

In a particular embodiment of the present invention, the immunocompromised subject to be vaccinated suffers from a disease selected from the groups consisting of: HIV-infection, acquired immunodeficiency syndrome (AIDS), cancer, chronic heart or lung disorders, congestive heart failure, diabetes mellitus, chronic liver disease, alcoholism, cirrhosis, spinal fluid leaks, cardiomyopathy, chronic bronchitis, emphysema, chronic obstructive pulmonary disease (COPD), spleen dysfunction (such as sickle cell disease), lack of spleen function (asplenia), blood malignancy, leukemia, multiple myeloma, Hodgkin's disease, lymphoma, kidney failure, nephrotic syndrome and asthma.

In an embodiment of the present invention, the immunocompromised subject to be vaccinated suffers from malnutrition.

In a particular embodiment of the present invention, the immunocompromised subject to be vaccinated is taking a drug or treatment that lowers the body's resistance to infection. In an embodiment, said drug is selected from the one disclosed on page 26, line 33, to page 26, line 4, of WO 2010/125480.

In a particular embodiment of the present invention, the immunocompromised subject to be vaccinated is a smoker.

In a particular embodiment of the present invention, the immunocompromised subject to be vaccinated has a white blood cell count (leukocyte count) below 5×10⁹ cells per liter, or below 4×10⁹ cells per liter, or below 3×10⁹ cells per liter, or below 2×10⁹ cells per liter, or below 1×10⁹ cells per liter, or below 0.5×10⁹ cells per liter, or below 0.3×10⁹ cells per liter, or below 0.1×10⁹ cells per liter.

White blood cell count (leukocyte count): The number of white blood cells (WBC) in the blood. The WBC is usually measured as part of the CBC (complete blood count). White blood cells are the infection-fighting cells in the blood and are distinct from the red (oxygen-carrying) blood cells known as erythrocytes. There are different types of white blood cells, including neutrophils (polymorphonuclear leukocytes; PMN), band cells (slightly immature neutrophils), T-type lymphocytes (T-cells), B-type lymphocytes (B-cells), monocytes, eosinophils, and basophils. All the types of white blood cells are reflected in the white blood cell count. The normal range for the white blood cell count is usually between 4,300 and 10,800 cells per cubic millimeter of blood. This can also be referred to as the leukocyte count and can be expressed in international units as 4.3-10.8×10⁹ cells per liter.

In a particular embodiment of the present invention, the immunocompromised subject to be vaccinated suffers from neutropenia. In a particular embodiment of the present invention, the immunocompromised subject to be vaccinated has a neutrophil count below 2×10⁹ cells per liter, or below 1×10⁹ cells per liter, or below 0.5×10⁹ cells per liter, or below 0.1×10⁹ cells per liter, or below 0.05×10⁹ cells per liter. A low white blood cell count or “neutropenia” is a condition characterized by abnormally low levels of neutrophils in the circulating blood. Neutrophils are a specific kind of white blood cell that help to prevent and fight infections. The most common reason that cancer patients experience neutropenia is as a side effect of chemotherapy. Chemotherapy-induced neutropenia increases a patient's risk of infection and disrupts cancer treatment.

In a particular embodiment of the present invention, the immunocompromised subject to be vaccinated has a CD4+ cell count below 500/mm³, or CD4+ cell count below 300/mm³, or CD4+ cell count below 200/mm³, CD4+ cell count below 100/mm³, CD4+ cell count below 75/mm³, or CD4+ cell count below 50/mm³.

CD4 cell tests are normally reported as the number of cells in mm³. Normal CD4 counts are between 500 and 1,600, and CD8 counts are between 375 and 1,100. CD4 counts drop dramatically in people with HIV.

In an embodiment of the invention, any of the immunocompromised subjects disclosed herein is a human male. In an embodiment of the invention, any of the immunocompromised subjects disclosed herein is a human female.

### 6 Regimen

In some cases, as little as one dose of the immunogenic composition according to the invention is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the invention is a single dose. In a particular embodiment, said single dose schedule is for healthy persons being at least 2 years of age.

In an embodiment, the schedule of vaccination of the immunogenic composition according to the invention is a multiple dose schedule. In a particular embodiment, said multiple dose schedule consists of a series of 2 doses separated by an interval of about 1 month to about 2 months. In a particular embodiment, said multiple dose schedule consists of a series of 2 doses separated by an interval of about 1 month, or a series of 2 doses separated by an interval of about 2 months.

In another embodiment, said multiple dose schedule consists of a series of 3 doses separated by an interval of about 1 month to about 2 months. In another embodiment, said multiple dose schedule consists of a series of 3 doses separated by an interval of about 1 month, or a series of 3 doses separated by an interval of about 2 months.

In another embodiment, said multiple dose schedule consists of a series of 3 doses separated by an interval of about 1 month to about 2 months followed by a fourth dose about 10 months to about 13 months after the first dose. In another embodiment, said multiple dose schedule consists of a series of 3 doses separated by an interval of about 1 month followed by a fourth dose about 10 months to about 13 months after the first dose, or a series of 3 doses separated by an interval of about 2 months followed by a fourth dose about 10 months to about 13 months after the first dose.

In an embodiment, the multiple dose schedule consists of at least one dose (e.g., 1, 2 or 3 doses) in the first year of age followed by at least one toddler dose.

In an embodiment, the multiple dose schedule consists of a series of 2 or 3 doses separated by an interval of about 1 month to about 2 months (for example 28-56 days between doses), starting at 2 months of age, and followed by a toddler dose at 12-18 months of age. In an embodiment, said multiple dose schedule consists of a series of 3 doses separated by an interval of about 1 month to about 2 months (for example 28-56 days between doses), starting at 2 months of age, and followed by a toddler dose at 12-months of age. In another embodiment, said multiple dose schedule consists of a series of 2 doses separated by an interval of about 2 months, starting at 2 months of age, and followed by a toddler dose at 12-18 months of age.

In an embodiment, the multiple dose schedule consists of a 4-dose series of vaccine at 2, 4, 6, and 12-15 months of age.

In an embodiment, a prime dose is given at day 0 and one or more boosts are given at intervals that range from about 2 to about 24 weeks, preferably with a dosing interval of 4-8 weeks.

In an embodiment, a prime dose is given at day 0 and a boost is given about 3 months later.

### 7. The Invention Also Provides the Following Embodiments as Defined in the Following Numbered Paragraphs 1 to 96

1. A method of making a capsular saccharide glycoconjugate, comprising the steps of:
(a) reacting an isolated capsular saccharide with a carbonic acid derivative and an azido linker in an aprotic solvent to produce an activated azido saccharide,
(b) reacting a carrier protein with an agent bearing an N-Hydroxysuccinimide (NHS) moiety and an alkyne group where the NHS moiety reacts with the amino groups to form an amide linkage thereby obtaining an alkyne functionalized carrier protein,
(c) reacting the activated azido saccharide of step (a) with the activated alkyne-carrier protein of step (b) by Cu⁺¹ mediated azide-alkyne cycloaddition reaction to form a glycoconjugate.
2. The method of paragraph 1 wherein, the isolated saccharide is sized before the activation step (a).
3. The method of paragraph 2 wherein, the isolated capsular saccharide is sized to a weight average molecular weight between 200 kDa and 800 kDa.
4. The method of any one of paragraphs 1-3 wherein, said carbonic acid derivative is selected from the group consisting of 1,1′-carbonyldiimidazole (CDI), 1,1′-carbonyl-di-(1,2,4-triazole) (CDT), disuccinimidyl carbonate (DSC) and N-hydroxysuccinimidyl chloroformate.
5. The method of any one of paragraphs 1-3 wherein, said carbonic acid derivative is 1,1′-carbonyldiimidazole (CDI).
6. The method of any one of paragraphs 1-3 wherein, said carbonic acid derivative is 1,1′-Carbonyl-di-(1,2,4-triazole) (CDT).
7. The method of any one of paragraphs 1-6 wherein said azido linker is a compound of formula (I),

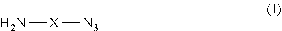

wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.
8. The method of any one of paragraphs 1-6 wherein said azido linker is a compound of formula (II),

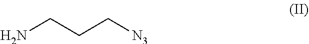

9. The method of any one of paragraphs 1-8 wherein, said agent bearing an N-Hydroxysuccinimide (NHS) moiety and an alkyne group is an agent bearing an N-Hydroxysuccinimide (NHS) moiety and a terminal alkyne.
10. The method of any one of paragraphs 1-8 wherein, said agent bearing an N-Hydroxysuccinimide (NHS) moiety and an alkyne group is an agent bearing an N-Hydroxysuccinimide (NHS) moiety and a cycloalkyne.
11. The method of any one of paragraphs 1-8 wherein, said agent bearing an N-Hydroxysuccinimide (NHS) moiety and an alkyne group is a compound of formula (III),

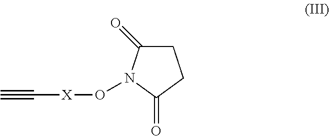

where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.
12. The method of any one of paragraphs 1-8 wherein, said agent bearing an N-Hydroxysuccinimide (NHS) moiety and an alkyne group is a compound of formula (IV):

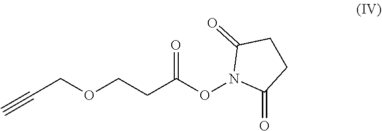 [SMILES: C#CCOCCC(=O)ON1C(=O)CCC1=O]

13. The method of any one of paragraphs 1-12 wherein, step a) comprises reacting the capsular saccharide with a carbonic acid derivative followed by reacting the carbonic acid derivative-activated capsular saccharide with an azido linker in an aprotic solvent to produce an activated azido capsular saccharide.
14. The method of any one of paragraphs 1-13 wherein, at step a) the isolated capsular saccharide is reacted with said carbonic acid derivative in an aprotic solvent.
15. The method of any one of paragraphs 1-13 wherein, at step a) the isolated capsular saccharide is reacted with a carbonic acid derivative in a solution consisting essentially of dimethylsulphoxide (DMSO).
16. The method of any one of paragraphs 1-14 wherein, at step a) the isolated capsular saccharide is reacted with CDI in an aprotic solvent comprising 0.1% to 1% (v/v) water.
17. The method of any one of paragraphs 1-14 wherein, at step a) the isolated capsular saccharide is reacted with CDI in DMSO comprising 0.1% to 1% (v/v) water.
18. The method of any one of paragraphs 1-17 wherein at step a) carbonic acid derivative activation is followed by the addition of water.
19. The method of paragraph 18 wherein water is added to bring the total water content in the mixture to between about 1% to about 10% (v/v).
20. The method of any one of paragraphs 1-19 wherein step a) further comprises reacting the carbonic acid derivative-activated saccharide with an amount of azido linker that is between 0.01-10 molar equivalent to the amount of saccharide Repeat Unit of the activated saccharide.
21. The method of any on of paragraphs 1-20 wherein the degree of activation of the activated saccharide following step a) is between 0.5 to 50%.
22. The method of any one of paragraphs 1-21 wherein step b) comprises reacting the carrier protein with an amount of agent bearing an N-Hydroxysuccinimide (NHS) moiety and an alkyne group that is 0.1-10 molar equivalents to the lysines on the carrier.
23. The method of any one of paragraphs 1-22 wherein the degree of activation of the activated carrier following step b) is between 1 and 50.
24. The method of any one of paragraphs 1-23 wherein the conjugation reaction c) is carried out in aqueous buffer in the presence of copper (1) as catalyst.
25. The method of any one of paragraphs 1-23 wherein the conjugation reaction c) is carried out in aqueous buffer in the presence of an oxidant and of copper (1) as catalyst.
26. The method of any one of paragraphs 1-23 wherein the conjugation reaction c) is carried out in aqueous buffer in the presence of copper (1) as catalyst and ascorbate as oxidant, wherein the reaction mixture further comprises THPTA (tris(3-hydroxypropyltriazolylmethyl)amine) and aminoguanidine.
27. The method of any one of paragraphs 1-26 wherein the initial input ratio (weight by weight) of activated azido saccharide to activated alkyne-carrier at setp c) is between 0.1 and 3.
28. The method of any one of paragraphs 1-27 wherein following step c), the method further comprises a step of capping the unreacted azido groups remained in the conjugate with an azido group capping agent.
29. The method of paragraph 28 wherein, said azido group capping agent is a compound of formula (V),

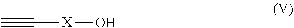

wherein X is (CH₂)ₙ wherein n is selected from 1 to 15.
30. The method of paragraph 28 wherein, said azido group capping agent is propargyl alcohol.
31. The method of any one of paragraphs 28-30 wherein the capping of the unreacted azido groups is performed with an amount of capping agent that is between 0.05 to 20 molar equivalents to the amount of saccharide repeat unit of the activated saccharide.
32. The method of any one of paragraphs 1-31 wherein following step c), the method further comprises a step of capping the unreacted alkyne groups remained in the conjugate with an alkyne group capping agent.
33. The method of paragraph 32 wherein said alkyne group capping agent is an agent bearing an azido group.
34. The method of paragraph 33 wherein said alkyne group capping agent is a compound of formula (VI),

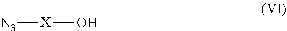

wherein X is (CH₂)ₙ wherein n is selected from 1 to 15.
35. The method of paragraph 32 wherein said alkyne group capping agent is 3-azido-1-propanol.
36. The method of any one of paragraphs 32-35 wherein the capping of the unreacted alkyne groups is performed with an amount of capping agent that is between 0.05 to 20 molar equivalents to the amount of saccharide repeat unit of the activated saccharide.
37. The method of any one of paragraphs 1-36 wherein the method further comprises the step of purifying the glycoconjugate after it is produced.
38. A capsular saccharide glycoconjugate produced according to any one of the methods of paragraphs 1 to 37.
39. A capsular saccharide glycoconjugate comprising a capsular saccharide covalently conjugated to a carrier protein (CP) through a spacer and having the general formula (VII):

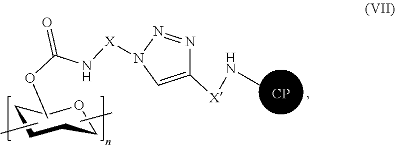

wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)ₙ, and O(CH₂CH₂O)ₘCH₂CH₂; where n′ is selected from 1 to 10 and m is selected from 1 to 4,
and wherein X is selected from the group consisting of CH₂O(CH₂)_{n′}CH₂C═O, CH₂O(CH₂CH₂O)_{m′}(CH₂)_{n′}CH₂C═O, where n″ is selected from 0 to 10 and m′ is selected from 0 to 4.
40. A capsular saccharide glycoconjugate comprising a capsular saccharide covalently conjugated to a carrier protein (CP) through a spacer and having the general formula (VII), wherein X is CH₂(CH₂)_{n′}, where n′ is 2 and wherein Xis CH₂O(CH₂)_{n″}CH₂C═O where n″ is 1.
41. A capsular saccharide glycoconjugate comprising a capsular saccharide covalently conjugated to a carrier protein (CP) through a spacer and having the general formula (VIII),

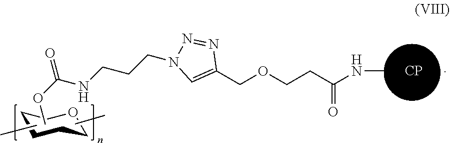

42. The capsular saccharide glycoconjugate of any one of paragraphs 38 to 41 comprising a capsular saccharide wherein the weight average molecular weight (Mw) of said capsular saccharide before conjugation is between 10 kDa and 2,000 kDa.
43. The capsular saccharide glycoconjugate of any one of paragraphs 38 to 41 comprising a capsular saccharide wherein the weight average molecular weight (Mw) of said capsular saccharide before conjugation is between 50 kDa and 1,000 kDa.
44. The capsular saccharide glycoconjugate of any one of paragraphs 38 to 41 comprising a capsular saccharide wherein the weight average molecular weight (Mw) of said capsular saccharide before conjugation is between 200 kDa and 750 kDa.
45. The capsular saccharide glycoconjugate of any one of paragraphs 38 to 44 having a weight average molecular weight (Mw) of between 250 kDa and 20,000 kDa.
46. The capsular saccharide glycoconjugate of any one of paragraphs 38 to 44 having a weight average molecular weight (Mw) of between 500 kDa and 5,000 kDa.
47. The capsular saccharide glycoconjugate of any one of paragraphs 38 to 44 having a weight average molecular weight (Mw) of between 750 kDa and 2,500 kDa.
48. The capsular saccharide glycoconjugate of any one of paragraphs 38 to 47 wherein, the degree of conjugation of the capsular saccharide glycoconjugate is between 2 and 15.
49. The capsular saccharide glycoconjugate of any one of paragraphs 38 to 48 wherein the ratio of capsular saccharide to carrier protein in the glycoconjugate (w/w) is between 0.5 and 3.0.
50. The capsular saccharide glycoconjugate of any one of paragraphs 38 to 49 wherein the said capsular saccharide glycoconjugate comprises at least one covalent linkage between the carrier protein and the saccharide for every 4 saccharide repeat units of the saccharide.
51. The capsular saccharide glycoconjugate of any one of paragraphs 38 to 49 wherein the said capsular saccharide glycoconjugate comprises at least one covalent linkage between the carrier protein and the saccharide for every 25 saccharide repeat units of the saccharide.
52. The capsular saccharide glycoconjugate of any one of paragraphs 38 to 49 wherein the said capsular saccharide glycoconjugate comprises at least one covalent linkage between the carrier protein and the saccharide for every 5 to 10 saccharide repeat units of the saccharide.
53. The capsular saccharide glycoconjugate of any one of paragraphs 38 to 56 wherein said carrier protein is CRM₁₉₇.
54. The capsular saccharide glycoconjugate of any one of paragraphs 38 to 53 wherein said carrier protein is SCP.
55. The capsular saccharide glycoconjugate of any one of paragraphs 38 to 53 wherein said carrier protein is an enzymatically inactive SCP.
56. The capsular saccharide glycoconjugate of any one of paragraphs 38 to 53 wherein said carrier protein is an enzymatically inactive SCP from GBS (SCPB).
57. The capsular saccharide glycoconjugate of any one of paragraphs 38 to 53 wherein said carrier protein is a fragment of an SCPB.
58. The capsular saccharide glycoconjugate of any one of paragraphs 38 to 53 wherein said carrier protein is a fragment of an SCP which comprises the protease domain, the protease-associated domain (PA domain) and the three fibronectin type Ill (Fn) domains but does not comprise the export signal presequence, the pro-sequence and the cell wall anchor domain.
59. The capsular saccharide glycoconjugate of any one of paragraphs 38 to 53 wherein said carrier protein is an enzymatically inactive fragment of an SCP, wherein said enzymatically inactive fragment of SCP comprises the protease domain, the protease-associated domain (PA domain) and the three fibronectin type Ill (Fn) domains but does not comprise the export signal presequence, the pro-sequence and the cell wall anchor domain.
60. The capsular saccharide glycoconjugate of any one of paragraphs 38 to 53 wherein said carrier protein is an enzymatically inactive fragment of SCP which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least one amino acid of the wild type sequence and wherein said replacement is selected from the group consisting of D130A, H193A, N295A and S512A where the numbers indicate the amino acid residue position in the peptidase according to the numbering of SEQ ID NO: 1 of WO00/34487.
61. The capsular saccharide glycoconjugate of any one of paragraphs 38 to 53 wherein said carrier protein is an enzymatically inactive fragment of SCP which comprises the protease domain, the protease-associated domain (PA domain) and the three fibronectin type Ill (Fn) domains but does not comprise the export signal presequence, the pro-sequence and the cell wall anchor domain, where said inactivation is accomplished by replacing at least two amino acids of the wild type sequence wherein said at least two amino acids replacements are D130A and S512A where the numbers indicate the amino acid residue position in the peptidase according to the numbering of SEQ ID NO: 1 of WO00/34487.
62. The capsular saccharide glycoconjugate of any one of paragraphs 38 to 53 wherein said carrier protein is an enzymatically inactive fragment of SCP consisting of a polypeptide having at least 95% identity with SEQ ID NO: 41.
63. The capsular saccharide glycoconjugate of any one of paragraphs 38 to 53 wherein said carrier protein is an enzymatically inactive fragment of SCP consisting of a polypeptide having at least 95% identity with SEQ ID NO: 42.
64. The capsular saccharide glycoconjugate of any one of paragraphs 38 to 53 wherein said carrier protein is an enzymatically inactive fragment of SCP which consists of SEQ ID NO: 41.
65. The capsular saccharide glycoconjugate of any one of paragraphs 38 to 53 wherein said carrier protein is an enzymatically inactive fragment of SCP which consists of SEQ ID NO: 42.
66. The capsular saccharide glycoconjugate of any one of paragraphs 38 to 53 wherein said carrier protein is DT (Diphtheria toxoid).
67. The capsular saccharide glycoconjugate of any one of paragraphs 38 to 53 wherein said carrier protein is TT (tetanus toxoid).
68. The capsular saccharide glycoconjugate of any one of paragraphs 38 to 53 wherein said carrier protein is PD (H. influenzae protein D).
69. The method of making a capsular saccharide glycoconjugate of any one of paragraphs 1 to 37 or the capsular saccharide glycoconjugate of any one of paragraphs 38 to 68, wherein said capsular saccharide is a capsular saccharide from a pathogenic bacteria.
70. The method of making a capsular saccharide glycoconjugate of any one of paragraphs 1 to 37 or the capsular saccharide glycoconjugate of any one of paragraphs 38 to 68, wherein said capsular saccharide is a capsular saccharide from a pathogenic Streptococcus, a pathogenic Staphylococcus, a pathogenic Enterococcus, a pathogenic Bacillus, a pathogenic Corynebacterium, a pathogenic Listeria, a pathogenic Erysipelothrix, a pathogenic Clostridium, a pathogenic Haemophilus, a pathogenic Neisseria or a pathogenic Escherichia.
71. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Enterococcus faecalis, Escherichia coli, Staphylococcus aureus or Streptococcus.
72. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Haemophilus influenzae, Neisseria meningitidis, S. pneumoniae, S. pyogenes, S. agalactiae, Group C & G Streptococci or Escherichia coli.
73. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Neisseria meningitidis, S. pneumoniae, S. agalactiae or Escherichia coli.
74. The method of making a capsular saccharide glycoconjugate of any one of paragraphs 1 to 37 or the capsular saccharide glycoconjugate of any one of paragraphs 38 to 68, wherein said capsular saccharide is a capsular saccharide from S. pneumoniae or S. agalactiae.
75. The method of making a capsular saccharide glycoconjugate of any one of paragraphs 1 to 37 or the capsular saccharide glycoconjugate of any one of paragraphs 38 to 68, wherein said capsular saccharide is a capsular saccharide from S. pneumoniae.
76. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Staphylococcus aureus.
77. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Enterococcus faecalis.
78. The method of making a capsular saccharide glycoconjugate of any one of paragraphs 1 to 37 or the capsular saccharide glycoconjugate of any one of paragraphs 38 to 68, wherein said capsular saccharide is a capsular saccharide from is Haemophilus influenzae type b.
79. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Neisseria meningitidis.
80. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Escherichia coli.
81. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Enterococcus faecalis.
82. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus agalactiae (Group B streptococcus (GBS)).
83. The method of making a capsular saccharide glycoconjugate of any one of paragraphs 1 to 37 or the capsular saccharide glycoconjugate of any one of paragraphs 38 to 68, wherein said capsular saccharide is a capsular saccharide from GBS type Ia, Ib, II, III, IV, V, VI, VII or VIII.
84. The method of making a capsular saccharide glycoconjugate of any one of paragraphs 1 to 37 or the capsular saccharide glycoconjugate of any one of paragraphs 38 to 68, wherein said capsular saccharide is a capsular saccharide from a Streptococcus pneumoniae serotype selected from the group consisting of serotypes 1, 2, 4, 5, 6A, 6B, 6C, 7C, 7F, 8, 9V, 9N, 10A, 10B, 11A, 12F, 14, 15A, 15B, 15C, 16F, 17F, 18C, 19A, 19F, 20, 21, 22A, 22F, 23A, 23B, 23F, 24B, 24F, 27, 29, 31, 33B, 33F, 34, 35B, 35F, 38, 72 and 73.
85. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 35B.
86. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 29.
87. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 15A.
88. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 24F.
89. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 23A.
90. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 23B.
91. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 6B.
92. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 19A.
93. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 19F.
94. The method of making a capsular saccharide glycoconjugate of any one of paragraphs 1 to 37 or the capsular saccharide glycoconjugate of any one of paragraphs 38 to 68, wherein said capsular saccharide is a capsular saccharide from Streptococcus pneumoniae serotype 23F.
95. The method of making a capsular saccharide glycoconjugate or the capsular saccharide glycoconjugate of any one of paragraphs 1 to 94, wherein said capsular saccharide is not a capsular saccharide from Streptococcus pneumoniae serotype 3.
96. An immunogenic composition comprising a capsular saccharide glycoconjugate of any one of paragraphs 38 to 95.

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLE

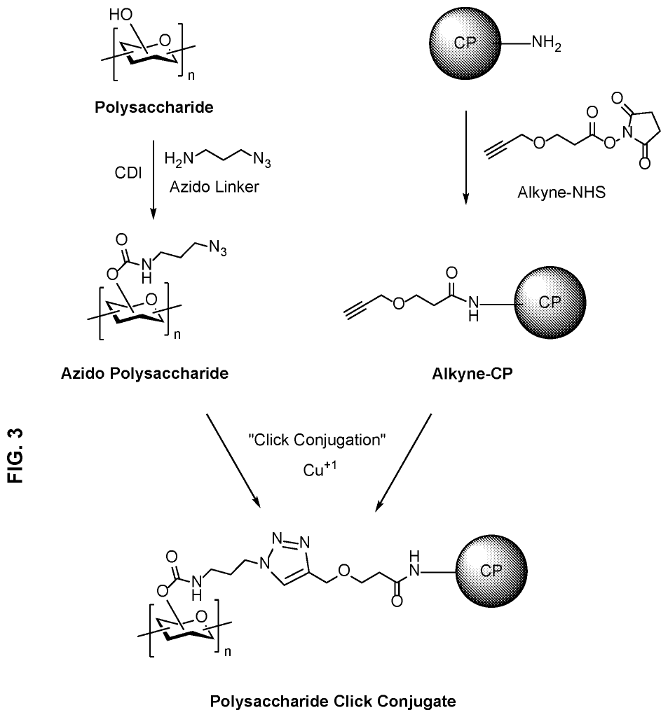

### Example 1: Conjugation of S. pneumoniae Serotype 35B Capsular Polysaccharide Using Click Chemistry (See FIG. 3)

1. Activation of Serotype 35B Capsular Polysaccharide with azido linker Serotype 35B capsular polysaccharide (200 mg) was mixed with imidazole (600 mg) and then frozen and lyophilized.

After 3 days lyophilization, the lyophilized polysaccharide was reconstituted with anhydrous DMSO (100 mL). The reaction mixture was then warmed to 45° C., and CDI (100 mg/mL in DMSO), 669 μL (2 MEq) was added. The reaction mixtures were stirred at 45° C. for 2 hrs. After the reaction mixtures were cooled to 23° C., WFI 2 mL (2% v/v) was added to quench free CDI and stirred for 30 min at 23° C. Then, 3-Azido-1-propylamine 41 μL (2 MEq) was added and then stirred at 23° C. for 20 hrs. After 20 hrs reaction, the reaction mixture was diluted to chilled (at 5° C.) with 400 mL 10 mM Sodium Phosphate Buffer (SPB) in saline (pH 7) (5×, v/v). The diluted reaction mixture was then purified by UF/DF using 10K MWCO PES membrane (Millipore Pellicon 2 Mini) against 10 mM SPB in saline (pH 7) (30×, v/v).
2. Activation of CRM₁₉₇ and SCP to alkyne-CRM₁₉₇ with Alkyne NHS ester Activation of CRM₁₉₇:

To the CRM₁₉₇ solution (1000 mg) WFI 57 mL and 0.5 M sodium phosphate buffer (pH 8.3) 50 mL were added. After cooled to 8° C., 3-Propargyloxy-propanoic acid NHS ester (POPS) (20 mg/mL in DMSO) 3.8 mL (0.5 MEq to lysine on CRM₁₉₇) was added to the reaction mixture dropwise maintaining the reaction temperature at 8±3° C. After the reaction mixture was stirred for 2 hrs at 8° C., purified by UF/DF using 10K MWCO PES membrane (Millipore Pellicon 2 Mini) against 100 mM sodium phosphate buffer in saline (pH 7.0) (30× diavolume). After UF/DF, sucrose 23 g (15% v/v) was added.

### Activation of SCP:

Activation of SCP was similar to activation of CRM₁₉₇ except that the amount of linker was adapted. The final amount of linker was 0.5 MEq to the SCP (0.092 w/w of linker per SCP).

### Click Conjugation:

Activated azido poly and alkyne CRM₁₉₇ or SCP is conjugated by Cu+1 mediated azide-alkyne cycloaddition reaction, referred as “Click Reaction

The mixture of 5 mM copper sulfate (CuSO₄) (1 mL) and 25 mM Tris(3-hydroxypropyltriazolylmethyl)amine (THPTA) (1 mL) were added to the mixture of Serotype 35B Capsular Polysaccharide activated with azido linker (see step 1 above) and alkyne-CRM₁₉₇ or alkyne-SCP (see step 2 above) (in 100 mM Sodium Phosphate Buffer (SPB) in saline, pH 7.0) at 23° C. and followed by the addition of 100 mM aminoguanidine (2 mL) and 100 mM sodium ascorbate (2 mL). After the reaction mixture was stirred for 2 hours at 23° C., the unreacted azido group was capped by propargyl alcohol (1 MEq) for 2 hours at 23° C. and after the first capping, subsequently the unreacted alkyne group was capped by 3-azido-1-propanol (2 MEq) for 2 hours at 23° C. Then, the reaction mixture was purified by UF/DF using 100K MWCO PES membrane against 10 mM EDTA+10 mM SPB in saline (pH 7.0) (30× diavolume) and then followed by 5 mM succinate in saline (pH 6.0) (30× diavolume).

TABLE 1
Attributes of Pn35B Conjugates obtained using click chemistry

| | | | |
|---|---|---|---|
| Conjugate # | 1 | 2 | 3 |

Polysaccharide

| | | | |
|---|---|---|---|
| Polysaccharide MW, kDa | 816 | 252 | 252 |

Activated polysaccharide attributes

| | | | |
|---|---|---|---|
| Degree of Activation (DoA) | ~18 | ~20 | ~26 |
| Activated Polysaccharide MW, kDa | 898 | 326 | 306 |

Activated carrier attributes

| | | | |
|---|---|---|---|
| Degree of Activation (DoA) | 15 | 17 | ~26 |
| Activated carrier MW, kDa | 58 | 58 | 101 |

Conjugate attributes

| | | | |
|---|---|---|---|
| Carrier protein | CRM₁₉₇ | CRM₁₉₇ | SCP |
| Yield (%) | 56 | 79 | 73 |
| SPR Ratio | 1.2 | 0.9 | 0.8 |
| Free Saccharide, % | 6 | <5 | <5 |
| Conjugate MW, kDa | 1729 | 1093 | 939 |

MW: molecular weight;
SPR: Saccharide to protein ratio

The click chemistry allows for generating 35B conjugates with very low free saccharide and a good yield. Furthermore, it allows for obtaining 35B conjugates comprising polysaccharides close to the native polysaccharides (no size reduction is observed during the activation step contrary to activation with periodarte, see WO 2020/247299).

### Example 2: Conjugation of S. pneumoniae Serotype 29 Capsular Polysaccharide Using Click Chemistry (See FIG. 3)

1. Activation of Serotype 29 Capsular Polysaccharide with azido linker Serotype 29 capsular polysaccharide (200 mg) was mixed with imidazole (600 mg) and then frozen and lyophilized.

After 3 days lyophilization, the lyophilized polysaccharide was reconstituted with anhydrous DMSO (100 mL). The reaction mixture was then warmed to 45° C., and CDI (100 mg/mL in DMSO), 1050 μL (3 MEq) was added. The reaction mixture was stirred (300 rpm) at 45° C. for 3 hrs. After the reaction mixture was cooled to 23° C., WFI 2 mL (2% v/v) was added to quench free CDI and stirred for 30 min at 23° C. Then, 3-Azido-1-propylamine 42 μL (2 MEq) was added and followed by triethylamine 60 μL and then stirred at 23° C. for 20 hrs. After 20 hrs reaction, the reaction mixture was diluted to chilled (at 5° C.) with 400 mL 10 mM Sodium Phosphate Buffer (SPB) in saline (pH 7) (5×, v/v). The diluted reaction mixture was then purified by UF/DF using 10K MWCO PES membrane (Millipore Pellicon 2 Mini) against 10 mM SPB in saline (pH 7) (30×, v/v).
2. Activation of CRM₁₉₇ to alkyne-CRM₁₉₇ with Alkyne NHS ester

To the CRM₁₉₇ solution (1000 mg) WFI 57 mL and 0.5 M sodium phosphate buffer (pH 8.3) 50 mL were added. After cooled to 8° C., 3-Propargyloxy-propanoic acid NHS ester (POPS) (20 mg/mL in DMSO) 3.8 mL (0.5 MEq to lysine on CRM₁₉₇) was added to the reaction mixture dropwise maintaining the reaction temperature at 8±3° C. After the reaction mixture was stirred for 2 hrs at 8° C., purified by UF/DF using 10K MWCO PES membrane (Millipore Pellicon 2 Mini) against 100 mM sodium phosphate buffer in saline (pH 7.0) (30× diavolume). After UF/DF, sucrose 23 g (15% v/v) was added.
3. Click Conjugation: Activated azido poly and alkyne CRM₁₉₇ is conjugated by Cu+1 mediated azide-alkyne cycloaddition reaction, referred as “Click Reaction

The mixture of 5 mM copper sulfate (CuSO₄) (1 mL) and 25 mM Tris(3-hydroxypropyltriazolylmethyl)amine (THPTA) (1 mL) were added to the mixture of Serotype 29 Capsular Polysaccharide activated with azido linker (see step 1 above) and alkyne-CRM₁₉₇ (see step 2 above) (in 100 mM Sodium Phosphate Buffer (SPB) in saline, pH 7.0) at 23° C. and followed by the addition of 100 mM aminoguanidine (2 mL) and 100 mM sodium ascorbate (2 mL). After the reaction mixture was stirred for 2 hours at 23° C., the unreacted azido group was capped by propargyl alcohol (1 MEq) for 2 hours at 23° C. and after the first capping, subsequently the unreacted alkyne group was capped by 3-azido-1-propanol (2 MEq) for 2 hours at 23° C. Then, the reaction mixture was purified by UF/DF using 100K MWCO PES membrane against 10 mM EDTA+10 mM SPB in saline (pH 7.0) (30× diavolume) and then followed by 5 mM succinate in saline (pH 6.0) (30× diavolume).

TABLE 2
Attributes of Pn29 Conjugates obtained using click chemistry

| | |
|---|---|
| Conjugate # | 1 |

Starting polysaccharide

| | |
|---|---|
| Polysaccharide MW, kDa | 674 |

Activated polysaccharide atributes

| | |
|---|---|
| Degree of Activation (DoA) | ~9 |
| Activated Polysaccharide MW, kDa | 755 |

Conjugate attributes

| | |
|---|---|
| Carrier Protein | CRM₁₉₇ |
| Yield (%) | 56 |
| SPR Ratio | 1.6 |
| Free Saccharide, % | 11 |
| Conjugate MW, kDa | 1165 |

MW: molecular weight;
SPR: Saccharide to protein ratio

The click chemistry allows for generating 29 conjugates with low free saccharide and a good yield. Furthermore, it allows for obtaining serotype 29 conjugates comprising polysaccharides close to the native polysaccharides (no size reduction is observed during the activation step contrary to activation with periodarte, see WO 2020/247299).

### Example 3. Immunogenicity of Serotype 35B Conjugate Conjugated Using Click Chemistry

The opsonophagocytic activity (OPA) titers for Serotype 35B conjugate to -CRM₁₉₇ or SCP in mice were determined under standard conditions. Click chemistry (Click) was used to generate the tested conjugates (see attributes at Table 1).

Groups of 6-8 weeks old female Swiss Webster mice were immunized (250 μL) with 0.01 μg/animal, 0.05 μg/animal or 0.1 μg/animal of test conjugates via the subcutaneous route on week 0. The mice were boosted with the same dose of conjugate on week 3 and then bled at week 5. Serotype-specific OPAs were performed on week 5 sera samples.

The results are presented at table 3.

TABLE 3
OPA titers following vaccination with Serotype 35B antigen conjugated
with Click to -CRM₁₉₇ or SCP. Conjugated Serotype 35B was used
to vaccinate mice in the presence of adjuvant. Female Swiss-Webster
mice, 6-8 weeks old; Doses: 0.01; 0.05 and 0.1 μg/animal +
AlPO4; Vaccinate: 0 and 3 wk.; exsanguinate wk. 5 Readout: OPA

| | Dose (μg/animal) | 0.01 | 0.05 | 0.1 |
|---|---|---|---|---|
| Click-CRM₁₉₇ | Mean (OPA titers) | 21 | 341 | 2013 |
| (Native) | Total # of mice | 45 | 25 | 46 |
| Conjugate #1 of | # of non-responders | 31 | 5 | 0 |
| Table 1 | % of non-responders | 69 | 20 | 0 |
| Click-CRM₁₉₇ | Mean (OPA titers) | NA | 44 | NA |
| (Sized) | Total # of mice | NA | 25 | NA |
| Conjugate #2 of | # of non-responders | NA | 14 | NA |
| Table 1 | % of non-responders | NA | 56 | NA |
| Click-SCP | Mean (OPA titers) | 27 | 182 | 752 |
| Conjugate #3 of | Total # of mice | 25 | 25 | 25 |
| Table 1 | # of non-responders | 13 | 5 | 3 |
| | % of non-responders | 52 | 20 | 12 |

NA: ‘Not Available’

The data of Table 3 indicate that all serotype 35B conjugates elicited dose dependent OPA titers in a murine immunogenicity model.

### Example 4: Conjugation of S. pneumoniae Serotypes 23A and 23B Capsular Polysaccharide Using Click Chemistry (See FIG. 3)

Serotypes 23A and 23B capsular polysaccharides have been activated with the azido linker (3-Azido-1-propylamine), using similar processes used for serotypes 35B and 29 (see Examples 1 and 2). Activation of CRM₁₉₇ and SCP to alkyne-CRM₁₉₇ or alkyne-SCP with Alkyne NHS ester, and click conjugation of the activated azido polysaccharides 23A and 23B with alkyne CRM₁₉₇ or SCP (conjugated by Cu+1 mediated azide-alkyne cycloaddition reaction), were conducted using similar processes of the ones of Examples 1 and 2.

The attributes of the conjugates which have been obtained are presented at Table 4 and 5.

TABLE 4
Attributes of Pn23A Conjugates obtained using click chemistry

| | | |
|---|---|---|
| Conjugate # | 1 | 2 |

Polysaccharide

| | | |
|---|---|---|
| Polysaccharide MW, kDa | 196 | 196 |

Activated polysaccharide attributes

| | | |
|---|---|---|
| Degree of Activation (DoA) | ~10-20 | ~10-20 |
| Activated Polysaccharide MW, kDa | 248 | 248 |

Activated carrier attributes

| | | |
|---|---|---|
| Degree of Activation (DoA) | 15 | 26 |
| Activated carrier MW, kDa | 65 | 99.3 |

Conjugate attributes

| | | |
|---|---|---|
| Carrier protein | CRM₁₉₇ | SCP |
| Yield (%) | 57 | 77 |
| SPR Ratio | 1.2 | 1 |
| Free Saccharide, % | <5 | <5 |
| Conjugate MW, kDa | 1469 | 1412 |

MW: molecular weight;
SPR: Saccharide to protein ratio

The click chemistry allows for generating 23A conjugates with very low free saccharide and a good yield.

TABLE 5
Attributes of Pn23B Conjugates obtained using click chemistry

| | | |
|---|---|---|
| Conjugate # | 1 | 2 |

Polysaccharide

| | | |
|---|---|---|
| Polysaccharide MW, kDa | 157 | 157 |

Activated polysaccharide attributes

| | | |
|---|---|---|
| Degree of Activation (DoA) | 18 | 18 |
| Activated Polysaccharide MW, kDa | 165 | 165 |

Activated carrier attributes

| | | |
|---|---|---|
| Degree of Activation (DoA) | 17 | 26 |
| Activated carrier MW, kDa | 58 | 101 |

Conjugate attributes

| | | |
|---|---|---|
| Carrier protein | CRM₁₉₇ | SCP |
| Yield (%) | 58 | 63 |
| SPR Ratio | 0.91 | 0.85 |
| Free Saccharide, % | 10.6 | 11.5 |
| Conjugate MW, kDa | 1113 | 919 |

MW: molecular weight;
SPR: Saccharide to protein ratio

The click chemistry allows for generating 23B conjugates with low % free saccharide and a good yield.

### Example 5: Conjugation of S. pneumoniae Serotype 15A Capsular Polysaccharide Using Click Chemistry (See FIG. 3)

Serotype 15A capsular polysaccharide has been activated with the azido linker (3-Azido-1-propylamine), using similar processes used for serotypes 35B and 29 (see Examples 1 and 2). Activation of CRM₁₉₇ and SCP to alkyne-CRM₁₉₇ or alkyne-SCP with Alkyne NHS ester, and click conjugation of the activated azido polysaccharide 15A with alkyne CRM₁₉₇ or SCP (conjugated by Cu+1 mediated azide-alkyne cycloaddition reaction), were conducted using similar processes of the ones of Examples 1 and 2.

The attributes of the conjugates which have been obtained are presented at Table 6.

TABLE 6
Attributes of Pn15A Conjugates obtained using click chemistry

| | | |
|---|---|---|
| Conjugate # | 1 | 2 |

Polysaccharide

| | | |
|---|---|---|
| Polysaccharide MW, kDa | 175 | 229 |

Activated polysaccharide attributes

| | | |
|---|---|---|
| Degree of Activation (DoA) | ~15 | ~17 |
| Activated Polysaccharide MW, kDa | 244 | 320 |

Activated carrier attributes

| | | |
|---|---|---|
| Degree of Activation (DoA) | 17 | ~26 |
| Activated carrier MW, kDa | 58 | 101 |

Conjugate attributes

| | | |
|---|---|---|
| Carrier protein | CRM₁₉₇ | SCP |
| Yield (%) | 25 | 64 |
| SPR Ratio | 1.0 | 1.1 |
| Free Saccharide, % | 6 | 9 |
| Conjugate MW, kDa | 2249 | 802 |

MW: molecular weight;
SPR: Saccharide to protein ratio

The click chemistry allows for generating 15A conjugates with low free saccharide and a good yield for SPC.

### Example 6: Conjugation of S. pneumoniae Serotype 24F Capsular Polysaccharide Using Click Chemistry (See FIG. 3)

Serotype 24F capsular polysaccharide has been activated with the azido linker (3-Azido-1-propylamine), using similar processes used for serotypes 35B and 29 (see Examples 1 and 2). Activation of CRM₁₉₇ and SCP to alkyne-CRM₁₉₇ or alkyne-SCP with Alkyne NHS ester, and click conjugation of the activated azido polysaccharide 24F with alkyne CRM₁₉₇ or SCP (conjugated by Cu+1 mediated azide-alkyne cycloaddition reaction), were conducted using similar processes of the ones of Examples 1 and 2.

The attributes of the conjugates which have been obtained are presented at Table 7.

TABLE 7
Attributes of Pn24F Conjugates obtained using click chemistry

| | | |
|---|---|---|
| Conjugate # | 1 | 2 |

Polysaccharide

| | | |
|---|---|---|
| Polysaccharide MW, kDa | 194 | 194 |

Activated polysaccharide attributes

| | | |
|---|---|---|
| Degree of Activation (DoA) | 6~10 | 6~10 |
| Activated Polysaccharide MW, kDa | 282 | 282 |

Activated carrier attributes

| | | |
|---|---|---|
| Degree of Activation (DoA) | 15 | 20 |
| Activated carrier MW, kDa | 65 | 101 |

Conjugate attributes

| | | |
|---|---|---|
| Carrier protein | CRM₁₉₇ | SCP |
| Yield (%) | 52 | 82 |
| SPR Ratio | 1.1 | 0.9 |
| Free Saccharide, % | 7 | <5 |
| Conjugate MW, kDa | 1195 | 2206 |

MW: molecular weight;
SPR: Saccharide to protein ratio

The click chemistry allows for generating 24F conjugates with low free saccharide and a good yield.

### Example 7: Conjugation of S. pneumoniae Serotype 6B Capsular Polysaccharide Using Click Chemistry (See FIG. 3)

Serotype 6B capsular polysaccharide has been activated with the azido linker (3-Azido-1-propylamine), using similar processes used for serotypes 35B and 29 (see Examples 1 and 2). Activation of CRM₁₉₇ and SCP to alkyne-CRM₁₉₇ or alkyne-SCP with Alkyne NHS ester, and click conjugation of the activated azido polysaccharide 6B with alkyne CRM₁₉₇ or SCP (conjugated by Cu+1 mediated azide-alkyne cycloaddition reaction), were conducted using similar processes of the ones of Examples 1 and 2.

The attributes of the conjugates which have been obtained are presented at Table 8.

TABLE 8
Attributes of 6B Conjugates obtained using click chemistry

| | | |
|---|---|---|
| Conjugate # | 1 | 2 |

Activated polysaccharide attributes

| | | |
|---|---|---|
| Degree of Activation (DoA) | 20 | 20 |

Activated carrier attributes

| | | |
|---|---|---|
| Degree of Activation (DoA) | 15 | 26 |

Conjugate attributes

| | | |
|---|---|---|
| Carrier protein | CRM₁₉₇ | SCP |
| SPR Ratio | 1.20 | 1.10 |
| Free Saccharide, % | <5 | <5 |
| Free Protein, % | <1 | <1 |
| Conjugate MW, kDa | 1919 | 1927 |

MW: molecular weight;
SPR: Saccharide to protein ratio

The click chemistry allows for generating 6B conjugates with very low free saccharide and free protein.

### Example 8: Conjugation of S. pneumoniae Serotype 19A Capsular Polysaccharide Using Click Chemistry (See FIG. 3)

Serotype 19A capsular polysaccharide has been activated with the azido linker (3-Azido-1-propylamine), using similar processes used for serotypes 35B and 29 (see Examples 1 and 2). Activation of CRM₁₉₇ and SCP to alkyne-CRM₁₉₇ or alkyne-SCP with Alkyne NHS ester, and click conjugation of the activated azido polysaccharide 19A with alkyne CRM₁₉₇ or SCP (conjugated by Cu+1 mediated azide-alkyne cycloaddition reaction), were conducted using similar processes of the ones of Examples 1 and 2.

The attributes of the conjugates which have been obtained are presented at Table 9.

TABLE 9
Attributes of 19A Conjugates obtained using click chemistry

| | | |
|---|---|---|
| Conjugate # | 1 | 2 |

Activated polysaccharide attributes

| | | |
|---|---|---|
| Degree of Activation (DoA) | 17 | 17 |

Activated carrier attributes

| | | |
|---|---|---|
| Degree of Activation (DoA) | 15 | 26 |

Conjugate attributes

| | | |
|---|---|---|
| Carrier protein | CRM₁₉₇ | SCP |
| SPR Ratio | 1.50 | 1.20 |
| Free Saccharide, % | 14 | <5 |
| Free Protein, % | <1 | <1 |
| Conjugate MW, kDa | 1360 | 994 |

MW: molecular weight;
SPR: Saccharide to protein ratio

The click chemistry allows for generating 19A conjugates with low free saccharide and very low free protein.

### Example 9: Conjugation of S. pneumoniae Serotype 19F Capsular Polysaccharide Using Click Chemistry (See FIG. 3)

Serotype 19F capsular polysaccharide has been activated with the azido linker (3-Azido-1-propylamine), using similar processes used for serotypes 35B and 29 (see Examples 1 and 2). Activation of CRM₁₉₇ and SCP to alkyne-CRM₁₉₇ or alkyne-SCP with Alkyne NHS ester, and click conjugation of the activated azido polysaccharide 19F with alkyne CRM₁₉₇ or SCP (conjugated by Cu+1 mediated azide-alkyne cycloaddition reaction), were conducted using similar processes of the ones of Examples 1 and 2.

The attributes of the conjugates which have been obtained are presented at Table 10.

TABLE 10
Attributes of 19F Conjugates obtained using click chemistry

| | | |
|---|---|---|
| Conjugate # | 1 | 2 |

Activated polysaccharide attributes

| | | |
|---|---|---|
| Degree of Activation (DoA) | 24 | 24 |

Activated carrier attributes

| | | |
|---|---|---|
| Degree of Activation (DoA) | 15 | 26 |

Conjugate attributes

| | | |
|---|---|---|
| Carrier protein | CRM₁₉₇ | SCP |
| SPR Ratio | 0.88 | 0.92 |
| Free Saccharide, % | 7 | 1 |
| Free Protein, % | <1 | <1 |
| Conjugate MW, kDa | 1775 | 2099 |

MW: molecular weight;
SPR: Saccharide to protein ratio

The click chemistry allows for generating 19F conjugates with low free saccharide and very low free protein.

### Example 10: Conjugation of S. pneumoniae Serotype 23F Capsular Polysaccharide Using Click Chemistry (See FIG. 3)

Serotype 23F capsular polysaccharide has been activated with the azido linker (3-Azido-1-propylamine), using similar processes used for serotypes 35B and 29 (see Examples 1 and 2). Activation of CRM₁₉₇ and SCP to alkyne-CRM₁₉₇ or alkyne-SCP with Alkyne NHS ester, and click conjugation of the activated azido polysaccharide 23F with alkyne CRM₁₉₇ or SCP (conjugated by Cu+1 mediated azide-alkyne cycloaddition reaction), were conducted using similar processes of the ones of Examples 1 and 2.

The attributes of the conjugates which have been obtained are presented at Table 11.

TABLE 11
Attributes of 23F Conjugates obtained using click chemistry

| | | |
|---|---|---|
| Conjugate # | 1 | 2 |

Activated polysaccharide attributes

| | | |
|---|---|---|
| Degree of Activation (DoA) | 26 | 28 |

Activated carrier attributes

| | | |
|---|---|---|
| Degree of Activation (DoA) | 15 | 26 |

Conjugate attributes

| | | |
|---|---|---|
| Carrier protein | CRM₁₉₇ | SCP |
| SPR Ratio | 1.20 | 1.10 |
| Free Saccharide, % | 5 | 8 |
| Free Protein, % | <1 | <1 |
| Conjugate MW, kDa | 2111 | 1798 |

MW: molecular weight;
SPR: Saccharide to protein ratio

### Example 11. Immunogenicity of Serotype 19F Conjugates Conjugated Using Click Chemistry

The opsonophagocytic activity (OPA) titers for Serotype 19F conjugate to -CRM₁₉₇ or SCP in mice were determined under standard conditions. Click chemistry (Click) was used to generate the tested conjugates (see attributes at Example 9, Table 10).

Groups of 6-8 weeks old female Swiss Webster mice were immunized (250 μL) with 0.05 μg/animal or 0.1 μg/animal of test conjugates via the subcutaneous route on week 0. The mice were boosted with the same dose of conjugate on week 3 and then bled at week 5. Serotype-specific OPAs were performed on week 5 sera samples.

The results are presented at table 12.

TABLE 12
OPA titers following vaccination with Serotype 19F antigen conjugated
with Click to -CRM₁₉₇ or SCP. Conjugated Serotype 19F was used
to vaccinate mice in the presence of adjuvant. Female Swiss-Webster
mice, 6-8 weeks old; Doses: 0.1, and 0.05 μg/animal +
AlPO4; Vaccinate: 0 and 3 wk.; exsanguinate wk. 5 Readout: OPA

| | Dose (μg/animal) | 0.05 | 0.1 |
|---|---|---|---|
| Click - CRM₁₉₇ | Mean | 102 | 143 |
| Conjugate #1 of | Total # of mice | 50 | 50 |
| Table 10 | # of non responders | 8 | 8 |
| | % of non responders | 16 | 16 |
| Click - SCP | Mean | 306 | 328 |
| Conjugate #2 of | Total # of mice | 50 | 50 |
| Table 10 | # of non responders | 0 | 1 |
| | % of non responders | 0 | 2 |

### Example 12. Immunogenicity of Serotype 29 Conjugates Conjugated Using Click Chemistry

The opsonophagocytic activity (OPA) titers for Serotype 29 conjugate to -CRM₁₉₇ in mice were determined under standard conditions. Click chemistry (Click) was used to generate the tested conjugate (see attributes at Example 2, Table 2).

Groups of 6-8 weeks old female Swiss Webster mice were immunized (250 μL) with 0.01 μg/animal, 0.05 μg/animal or 0.1 μg/animal of test conjugates via the subcutaneous route on week 0. The mice were boosted with the same dose of conjugate on week 3 and then bled at week 5. Serotype-specific OPAs were performed on week 5 sera samples.

The results are presented at table 13.

TABLE 13
OPA titers following vaccination with Serotype 29 antigen conjugated
to -CRM₁₉₇, with Click chemistry. Conjugated Serotype 29 was used
to vaccinate mice in the presence of adjuvant. Female Swiss-Webster
mice, 6-8 weeks old; Doses: 0.01; 0.05 and 0.1 μg/animal +
AlPO4; Vaccinate: 0 and 3 wk.; exsanguinate wk. 5 Readout: OPA

| | Dose (μg/animal) | 0.01 | 0.05 | 0.1 |
|---|---|---|---|---|
| Click-CRM₁₉₇ | Mean (OPA titers) | 28 | 1332 | 2271 |
| | Total # of mice | 25 | 25 | 25 |
| | # of non-responders | 12 | 1 | 0 |
| | % of non-responders | 48 | 4 | 0 |

The data of Table 13 indicate that the serotype 29 conjugate elicited dose dependent OPA titers in a murine immunogenicity model.

### Example 13. Immunogenicity of Serotype 19A Conjugates Conjugated Using Click Chemistry

The opsonophagocytic activity (OPA) titers for Serotype 19A conjugate to -CRM₁₉₇ or SCP in mice were determined under standard conditions. Click chemistry (Click) was used to generate the tested conjugates (see attributes at Example 8, Table 9).

Groups of 6-8 weeks old female Swiss Webster mice were immunized (250 μL) with 0.05 μg/animal or 0.1 μg/animal of test conjugates via the subcutaneous route on week 0. The mice were boosted with the same dose of conjugate on week 3 and then bled at week 5. Serotype-specific OPAs were performed on week 5 sera samples.

The results are presented at table 14.

TABLE 14
OPA titers following vaccination with Serotype 19A antigen conjugated
with Click to -CRM₁₉₇ or SCP. Conjugated Serotype 19A was used
to vaccinate mice in the presence of adjuvant. Female Swiss-Webster
mice, 6-8 weeks old; Doses: 0.05 and 0.1 μg/animal + AlPO4;
Vaccinate: 0 and 3 wk.; exsanguinate wk. 5 Readout: OPA

| | Dose (μg/animal) | 0.05 | 0.1 |
|---|---|---|---|
| Click-CRM₁₉₇ | Mean | 25 | 40 |
| Conjugate #1 of | Total # of mice | 50 | 49 |
| Table 9 | # of non responders | 24 | 19 |
| | % of non responders | 48 | 39 |
| Click-SCP | Mean | 126 | 327 |
| Conjugate #2 of | Total # of mice | 50 | 50 |
| Table 9 | # of non responders | 4 | 2 |
| | % of non responders | 8 | 4 |

### Example 14. Immunogenicity of Serotype 6B Conjugates Conjugated Using Click Chemistry

The opsonophagocytic activity (OPA) titers for Serotype 6B conjugate to -CRM₁₉₇ or SCP in mice were determined under standard conditions. Click chemistry (Click) was used to generate the tested conjugates (see attributes at Example 7, Table 8).

Groups of 6-8 weeks old female Swiss Webster mice were immunized (250 μL) with 0.05 μg/animal, 0.1 μg/animal or 0.2 μg/animal of test conjugates via the subcutaneous route on week 0. The mice were boosted with the same dose of conjugate on week 3 and then bled at week 5. Serotype-specific OPAs were performed on week 5 sera samples.

The results are presented at table 15.

TABLE 15
OPA titers following vaccination with Serotype 6B antigen conjugated
with Click to -CRM₁₉₇ or SCP. Conjugated Serotype 6B was used to
vaccinate mice in the presence of adjuvant. Female Swiss-Webster
mice, 6-8 weeks old; Doses: 0.2, 0.1, and 0.05 μg/animal +
AlPO4; Vaccinate: 0 and 3 wk.; exsanguinate wk. 5 Readout: OPA

| | Dose (μg/animal) | 0.05 | 0.1 | 0.2 |
|---|---|---|---|---|
| Click - CRM₁₉₇ | Mean | 76 | 255 | 217 |
| Conjugate #1 of | Total # of mice | 50 | 50 | 50 |
| Table 8 | # of non responders | 15 | 9 | 7 |
| | % of non responders | 30 | 18 | 14 |
| Click - SCP | Mean | 54 | 70 | 202 |
| Conjugate #2 of | Total # of mice | 48 | 50 | 49 |
| Table 8 | # of non responders | 18 | 16 | 7 |
| | % of non responders | 38 | 32 | 14 |

### Example 15. Immunogenicity of Serotype 15A Conjugates Conjugated Using Click Chemistry

The opsonophagocytic activity (OPA) titers for Serotype 15A conjugate to -CRM₁₉₇ or SCP in mice were determined under standard conditions. Click chemistry (Click) was used to generate the tested conjugates (see attributes at Example 5, Table 6).

Groups of 6-8 weeks old female Swiss Webster mice were immunized (250 μL) with 0.01 μg/animal, 0.1 μg/animal or 1.0 μg/animal of test conjugates via the subcutaneous route on week 0. The mice were boosted with the same dose of conjugate on week 3 and then bled at week 5. Serotype-specific OPAs were performed on week 5 sera samples.

The results are presented at table 16.

TABLE 16
OPA titers following vaccination with Serotype 15A antigen conjugated
with Click to -CRM₁₉₇ or SCP. Conjugated Serotype 15A was used
to vaccinate mice in the presence of adjuvant. Female Swiss-Webster
mice, 6-8 weeks old; Doses: 1.0, 0.1, and 0.01 μg/animal +
AlPO4; Vaccinate: 0 and 3 wk.; exsanguinate wk. 5 Readout: OPA

| | Dose (μg/animal) | 0.01 | 0.1 | 1.0 |
|---|---|---|---|---|
| Click - CRM₁₉₇ | Mean | 135 | 6457 | 16624 |
| Conjugate #1 of | Total # of mice | 23 | 25 | 25 |
| Table 6 | # of non responders | 5 | 0 | 0 |
| | % of non responders | 22 | 0 | 0 |
| Click - SCP | Mean | 463 | 3294 | 5578 |
| Conjugate #2 of | Total # of mice | 25 | 25 | 25 |
| Table 6 | # of non responders | 2 | 0 | 1 |
| | % of non responders | 8 | 0 | 4 |

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. A method of making a S. pneumoniae capsular saccharide glycoconjugate, comprising the steps of:
(a) reacting an isolated S. pneumoniae capsular saccharide with a carbonic acid derivative and an azido linker in an aprotic solvent to produce an activated azido saccharide,
(b) reacting a carrier protein with an agent bearing an N-Hydroxysuccinimide (NHS) moiety and an alkyne group where the NHS moiety reacts with an amino group of said carrier protein to form an amide linkage thereby obtaining an alkyne-functionalized carrier protein,
(c) reacting the activated azido saccharide of step (a) with the alkyne-functionalized carrier protein of step (b) by a Cu⁺¹ mediated azide-alkyne cycloaddition reaction to form a glycoconjugate.

2. (canceled)

3. The method of claim 1 wherein, said carbonic acid derivative is selected from the group consisting of 1,1′-carbonyldiimidazole (CDI), 1,1′-carbonyl-di-(1,2,4-triazole) (CDT), disuccinimidyl carbonate (DSC) and N-hydroxysuccinimidyl chloroformate.

4. The method of claim 1 wherein said azido linker is a compound of formula (I): 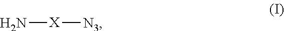
wherein X is selected from the group consisting of CH₂(CH₂)ₙ, (CH₂CH₂O)ₘCH₂CH₂, NHCO(CH₂)ₙ, NHCO(CH₂CH₂O)ₘCH₂CH₂, OCH₂(CH₂)ₙ and O(CH₂CH₂O)ₘCH₂CH₂; where n is selected from 1 to 10 and m is selected from 1 to 4.

5. The method of claim 4 wherein said azido linker is a compound of formula (II):

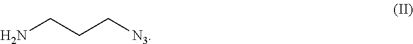

6. The method of claim 1 wherein, the alkyne group of said agent bearing an N-Hydroxysuccinimide (NHS) moiety and an alkyne group is a terminal alkyne or a cycloalkyne.

7. (canceled)

8. The method of claim 1 wherein, said agent bearing an N-Hydroxysuccinimide (NHS) moiety and an alkyne group is a compound of formula (III): 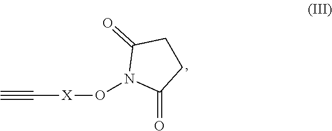
where X is selected from the group consisting of CH₂O(CH₂)ₙCH₂C═O and CH₂O(CH₂CH₂O)ₘ(CH₂)ₙCH₂C═O, where n is selected from 0 to 10 and m is selected from 0 to 4.

9. The method of claim 8 wherein, said agent bearing an N-Hydroxysuccinimide (NHS) moiety and an alkyne group is a compound of formula (IV):

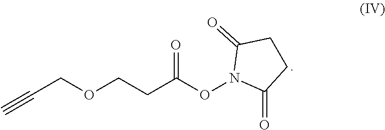 [SMILES: C#CCOCCC(=O)ON1C(=O)CCC1=O]

10. (canceled)

11. The method of claim 3 wherein, at step a) the isolated capsular saccharide is reacted with CDI in an aprotic solvent comprising 0.1% to 1% (v/v) water.

12-15. (canceled)

16. A S. pneumoniae capsular saccharide glycoconjugate produced according to the method of claim 1.

17. A S. pneumoniae capsular saccharide glycoconjugate comprising a S. pneumoniae capsular saccharide covalently conjugated to a carrier protein (CP) through a spacer and having the general formula (VII): 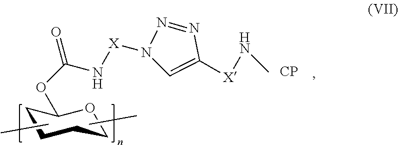
wherein X is selected from the group consisting of CH₂(CH₂)_{n′}, (CH₂CH₂O)ₘCH₂CH₂, NHCO(CH₂)_{n′}, NHCO(CH₂CH₂O)ₘCH₂CH₂, OCH₂(CH₂)_{n′} and O(CH₂CH₂O)ₘCH₂CH₂; where n′ is selected from 1 to 10 and m is selected from 1 to 4,
and wherein X′ is selected from the group consisting of CH₂O(CH₂)_{n″}CH₂C═O, CH₂O(CH₂CH₂O)_{m′}(CH₂)_{n″}CH₂C═O, where n″ is selected from 0 to 10 and m′ is selected from 0 to 4.

18. The S. pneumoniae capsular saccharide glycoconjugate of claim 17, wherein X is CH₂(CH₂)_{n′}, where n′ is 2 and wherein X′ is CH₂O(CH₂)_{n″}CH₂C═O where n″ is 1.

19. The S. pneumoniae capsular saccharide glycoconjugate of claim 17, wherein said S. pneumoniae capsular saccharide glycoconjugate has the general formula (VIII):

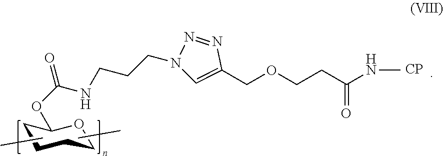

20-24. (canceled)

25. The S. pneumoniae capsular saccharide glycoconjugate of claim 17, wherein said carrier protein is selected from the group consisting of CRM₁₉₇, SCP, DT (Diphtheria toxoid) TT, (tetanus toxoid) and PD (H. influenzae protein D).

26. (canceled)

27. The method of claim 1, wherein the serotype of said S. pneumoniae capsular saccharide is selected from the group of S. pneumoniae serotypes consisting of 1, 2, 4, 5, 6A, 6B, 6C, 7C, 7F, 8, 9V, 9N, 10A, 10B, 11A, 12F, 14, 15A, 15B, 15C, 16F, 17F, 18C, 19A, 19F, 20, 21, 22A, 22F, 23A, 23B, 23F, 24B, 24F, 27, 29, 31, 33B, 33F, 34, 35B, 35F, 38, 72 and 73.

28. The method of claim 1, wherein the serotype of said S. pneumoniae capsular saccharide is other than serotype 3.

29. An immunogenic composition comprising a S. pneumoniae capsular saccharide glycoconjugate of claim 17.

30. The S. pneumoniae capsular saccharide glycoconjugate of claim 17, wherein the serotype of said S. pneumoniae capsular saccharide is selected from the group of S. pneumoniae serotypes consisting of 1, 2, 4, 5, 6A, 6B, 6C, 7C, 7F, 8, 9V, 9N, 10A, 10B, 11A, 12F, 14, 15A, 15B, 15C, 16F, 17F, 18C, 19A, 19F, 20, 21, 22A, 22F, 23A, 23B, 23F, 24B, 24F, 27, 29, 31, 33B, 33F, 34, 35B, 35F, 38, 72 and 73.

31. The S. pneumoniae capsular saccharide glycoconjugate of claim 17, wherein the serotype of said S. pneumoniae capsular saccharide is other than serotype 3.

32. The S. pneumoniae capsular saccharide glycoconjugate of claim 25, wherein said carrier protein is CRM197.

33. The S. pneumoniae capsular saccharide glycoconjugate of claim 25, wherein said carrier protein is SCP.
